(19) United States
(12) Patent Application Publication
(10) Pub. No.: US 2024/0425464 A1
(43) Pub. Date: Dec. 26, 2024

(54) Substituted Monohydrazides as Potent Antifungal Agents

(71) Applicant: University of Kentucky Research Foundation, Lexington, KY (US)

(72) Inventors: Sylvie Garneau-Tsodikova, Lexington, KY (US); Nishad Thamban Chandrika, Lexington, KY (US); Keith D. Green, Lexington, KY (US); Abbygail C. Spencer, Lexington, KY (US); Oleg V. Tsodikov, Lexington, KY (US)

(21) Appl. No.: 18/743,737

(22) Filed: Jun. 14, 2024

Related U.S. Application Data
(60) Provisional application No. 63/521,061, filed on Jun. 14, 2023.

(51) Int. Cl. C07D 241/24; A01N 37/18; A01N 43/40; A01N 43/60; A01P 3/00; A61K 31/166; A61K 31/4402; A61K 31/4965; A61P 31/10; C07D 213/56

(52) U.S. Cl. CPC C07D 241/24; A01N 37/18; A01N 43/40; A01N 43/60; A01P 3/00; A61K 31/166; A61K 31/4402; A61K 31/4965; A61P 31/10; C07D 213/56

(57) ABSTRACT

Compounds and compositions having antifungal activity, and methods of using the antifungal compounds and compositions, are described for use in treating fungal infections.

### RELATED APPLICATIONS

This application claims priority from U.S. Provisional Application Ser. No. 63/521,061 filed Jun. 14, 2023, the entire disclosure of which is incorporated herein by this reference.

### GOVERNMENT INTEREST

This invention was made with government support under grant number S10OD28690 awarded by the National Institutes of Health. The government has certain rights in the invention.

### TECHNICAL FIELD

The presently-disclosed subject matter generally relates to antifungal agents. In particular, embodiments of the presently-disclosed subject matter relate to substituted monohydrazide compounds useful as antifungal agents and methods for synthesizing and using the same to treat a fungal infection.

### INTRODUCTION

For the past two decades, fungal infections including superficial or mucosal and severe systemic invasive fungal infections (IFIs) have been on the rise, posing a significant threat to human health.^{1,2} Opportunistic fungal pathogens such as Candida species, Aspergillus species, and Cryptococcus species are responsible for the majority of IFIs. IFIs are a significant cause of morbidity and mortality, particularly for patients with compromised immune function.^{3,4} The increasing number of immunocompromised individuals, including cancer patients, organ transplant recipients, individuals infected with HIV, patients with diabetes and chronic obstructive pulmonary diseases, and the increasing elderly population has caused a surge in diseases caused by fungi in recent decades.⁵⁻⁸

The extensive and repeated use of the existing classes of antifungal drugs resulted in the resistance of fungal strains to available drug therapies.⁹⁻¹³ In addition, Candida auris, first identified in 2009 in Asia, has quickly become a cause of severe recalcitrant infections and deaths around the world.¹⁴⁻¹⁷ C. auris is often multidrug-resistant, causing outbreaks among hospital patients and nursing home residents.¹⁸⁻²⁰ In March 2023, the Centers for Disease Control and Prevention (CDC) issued a press release, which underscores a rapid spread of C. auris infections throughout healthcare facilities in the US, tripling in the numbers of clinical cases during a recent two-year span.²¹

It is clear that the current antifungal armamentarium is insufficient to stem the spread of C. auris infections. In order to combat the increasing cases of fungal infections, new antifungal drugs are needed that are broad spectrum and less toxic that the currently available agents.

Despite the increasing threat of spread of antimicrobial-resistant fungi, treatment options remain limited.^{22,23} The three available classes of current antifungals: the azoles, such as fluconazole (FLC) and voriconazole (VRC), the echinocandins, such as caspofungin (CFG), and the polyenes, such as amphotericin B (AmB) are still staple treatments of IFIs. Overreliance on these agents and their excessive use have caused an increase in the percentage of fungal species resistant to these classes of drugs.^{24,25} IFIs have also emerged as the leading co-infection in patients hospitalized with severe COVID-19 infections.²⁶⁻²⁹ The cost of antimicrobial resistance is immense, both for the economy and the human health.³⁰⁻³² The narrow antifungal activity, limited efficacy, significant side effects, drug-drug interactions, and toxicity associated with existing antifungal drugs highlight the urgent need to develop unique antifungal therapeutic agents.³³⁻³⁵

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds having antifungal activity, compositions including such compounds, methods of using such compounds, and methods of making such compounds. In some embodiments, the presently-disclosed subject matter includes a compound having the structure of formula (I) or a pharmaceutically acceptable salt thereof:

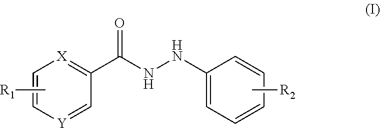

in which R₁ is selected from the group consisting of H, 5-Br, 6-Br, 5-F, 6-F, 2,4-diF, 3,5-diF, and 3,6-diF; R₂ is selected from the group consisting of 3-OMe, 4-OMe, 3-Cl, 4-Cl, 3,5-diCl, 3-F, 4-F, 2,4-diF, 2,5-diF, and 3,5-diF; and X and Y are independently selected from the group consisting of C and N.

In some embodiments, the compound has the formula selected from the group consisting of:

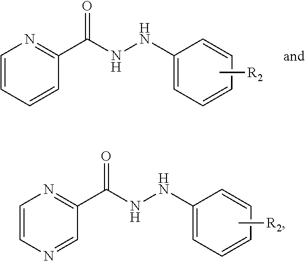

in which R₂ is selected from the group consisting of 3-OMe, 4-OMe, 3-Cl, 4-Cl, 3,5-diCl, 3-F, 4-F, 2,4-diF, 2,5-diF, and 3,5-diF.

In some embodiments, the compound has the formula selected from the group consisting of:

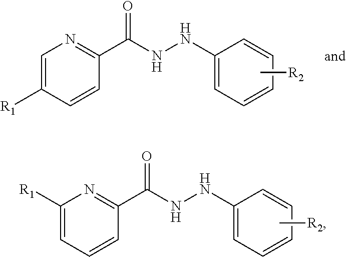

in which R₁ is selected from the group consisting of Br and F; and R₂ is selected from the group consisting of 3-OMe, 4-OMe, 3-Cl, 4-Cl, 3,5-diCl, 3-F, 4-F, 2,4-diF, 2,5-diF, and 3,5-diF.

In some embodiments, the compound has the formula selected from the group consisting of:

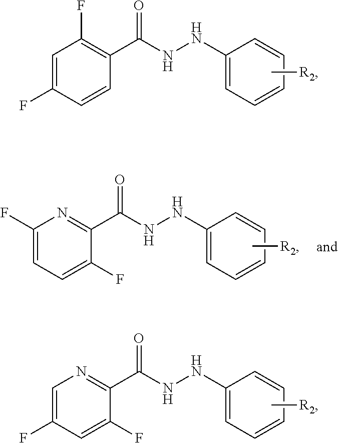

in which R₂ is selected from the group consisting of 3-OMe, 4-OMe, 3-Cl, 4-Cl, 3,5-diCl, 3-F, 4-F, 2,4-diF, 2,5-diF, and 3,5-diF.

The presently-disclosed subject matter further includes methods for synthesizing compounds useful as antifungal agents.

The presently-disclosed subject matter further includes compositions including a compound as disclosed herein and a suitable pharmaceutical carrier. In some embodiments, the composition includes at least two compounds as disclosed herein.

The presently-disclosed subject matter further includes methods of treating a fungal infection in a subject, comprising administering to the subject an effective amount of a compound or composition as disclosed herein. In some embodiments, the subject is a human subject. In some embodiments, the subject is a plant or crop.

### BRIEF DESCRIPTION OF THE DRAWINGS

The unique features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Synthetic scheme for the preparation of compounds disclosed herein, with chemical yields are provided in parentheses.

FIG. 2. Summary of structure-activity relationship (SAR) for antifungal activity of compounds disclosed herein.

FIG. 3A-3C. Two-dimensional (2D) bar graphs normalized at 100% depicting the dose-dependent cytotoxic activity of monohydrazides 2a, 2b. 2d, 2g, 2i, 9a, 9b, 9c, 9f, and 9g, as well as AmB and VRC against FIG. 3A: 3774A.1, FIG. 3B: HEK-293, and FIG. 3C: HepG2 mammalian cell lines.

FIG. 4. Time-kill curves for C. albicans American Type Culture Collection (ATCC) 10231 (strain A) with AmB (018 μg ml:¹), 2b (1×MIC), 2b (4×MIC), and untreated.

FIG. 5A-5B. Effect of compounds 2a-2j as well as AmB on FIG. 5A: disruption of pre-formed biofilms and FIG. 5 B: prevention of biofilm formation using C. albicans ATCC 10231 (strain A).

FIG. 6. Graph showing the minimum inhibitory concentration (MIC) values over 15 passages for compounds 2a (circles) and 2b (inverted triangles} against C. albicans ATCC 10231 (strain A).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds having antifungal activity, compositions including such compounds, methods of using such compounds, and methods of making such compounds. Unique monohydrazides disclosed herein display broad-spectrum activity against various fungal strains, including a panel of clinically relevant Candida auris strains. The activity of these compounds was either comparable or superior to amphotericin B against most of the fungal strains tested. These compounds possessed fungistatic activity in a time-kill assay and exhibited no mammalian cell toxicity. In addition, they prevented the formation of fungal biofilms. Even after repeated exposures, the Candida albicans ATCC 10231 (strain A) fungal strain did not develop resistance to these monohydrazides.

In some embodiments, the presently-disclosed subject matter includes a compound having the structure of formula (I) or a pharmaceutically acceptable salt thereof:

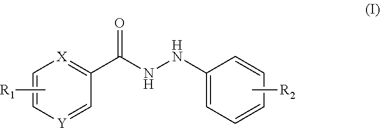

in which R₁ is selected from the group consisting of H, 5-Br, 6-Br, 5-F, 6-F, 2,4-diF, 3,5-diF, and 3,6-diF; R₂ is selected from the group consisting of 3-OMe, 4-OMe, 3-Cl, 4-Cl, 3,5-diCl, 3-F, 4-F, 2,4-diF, 2,5-diF, and 3,5-diF; and X and Y are independently selected from the group consisting of C and N.

In some embodiments, the compound has the formula selected from the group consisting of:

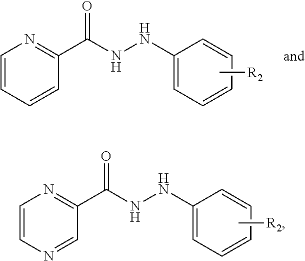

in which R₂ is selected from the group consisting of 3-OMe, 4-OMe, 3-Cl, 4-Cl, 3,5-diCl, 3-F, 4-F, 2,4-diF, 2,5-diF, and 3,5-diF.

In some embodiments, the compound has the formula selected from the group

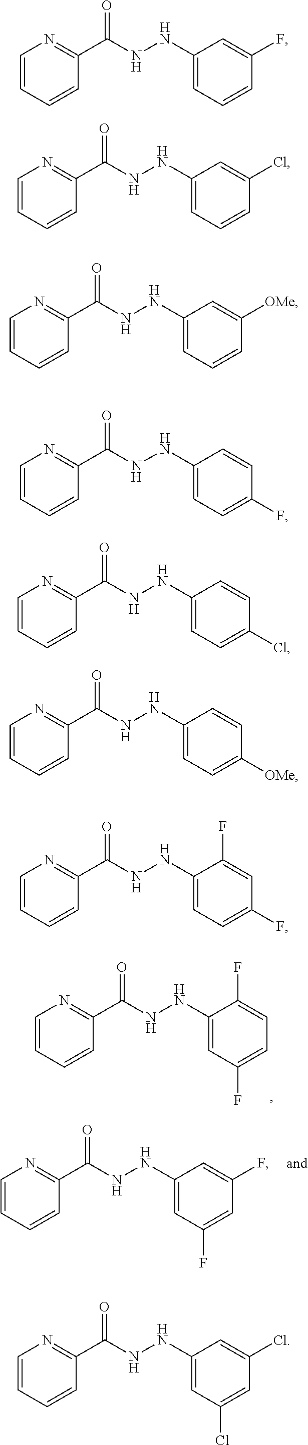

In some embodiments, the compound has the formula selected from the group

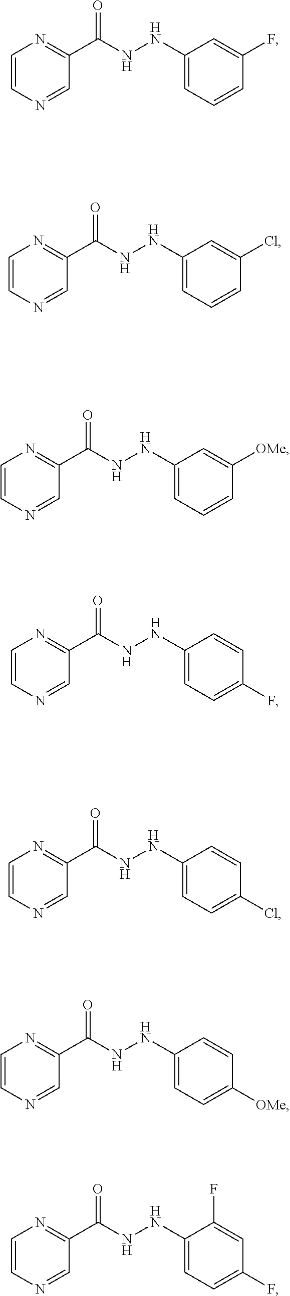

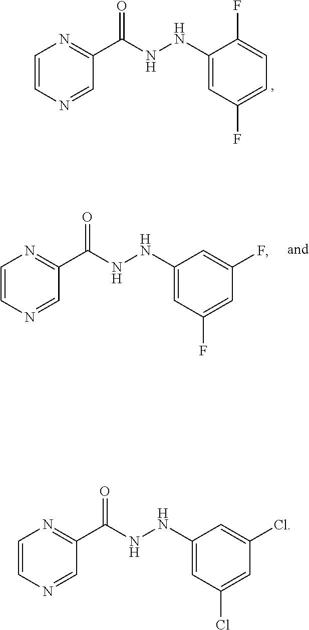 [SMILES: O=C(NNc1cc(F)ccc1F)c1cnccn1 ; O=C(NNc1cc(F)cc(F)c1)c1cnccn1 ; O=C(NNc1cc(Cl)cc(Cl)c1)c1cnccn1]

In some embodiments, the compound has the formula selected from the group

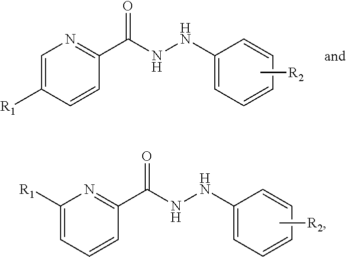

in which R₁ is selected from the group consisting of Br and F; and R₂ is selected from the group consisting of 3-OMe, 4-OMe, 3-Cl, 4-Cl, 3,5-diCl, 3-F, 4-F, 2,4-diF, 2,5-diF, and 3,5-diF.

In some embodiments, the compound has the formula selected from the group consisting of:

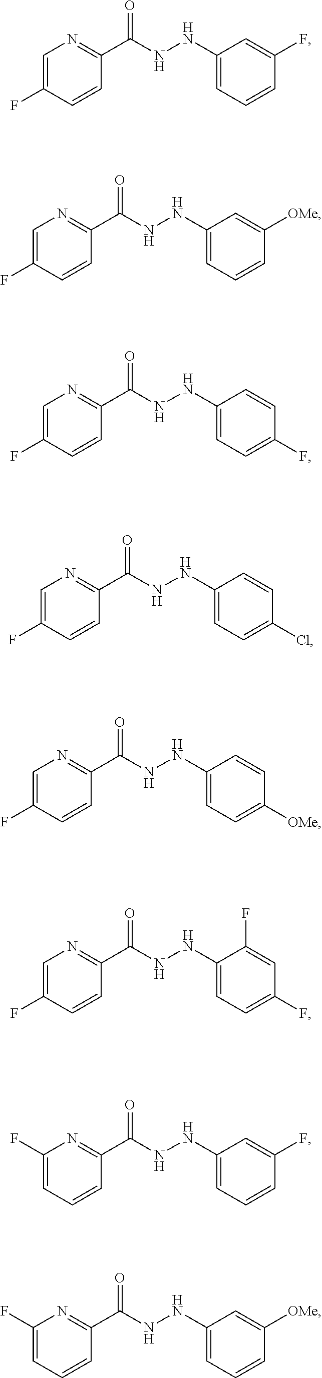

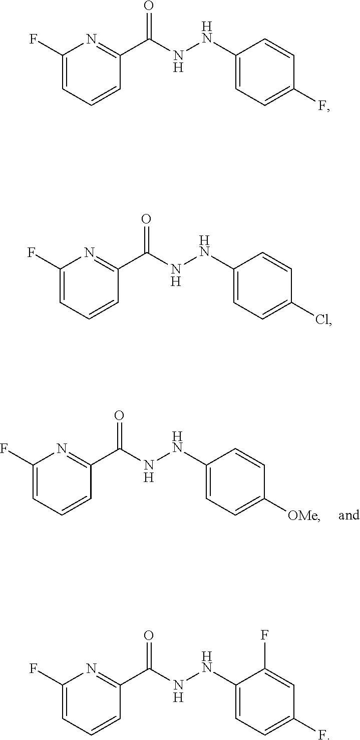 [SMILES: O=C(NNc1ccc(F)cc1)c1cccc(F)n1 ; O=C(NNc1ccc(Cl)cc1)c1cccc(F)n1 ; COc1ccc(NNC(=O)c2cccc(F)n2)cc1 ; O=C(NNc1ccc(F)cc1F)c1cccc(F)n1]

In some embodiments, the compound has the formula selected from the group

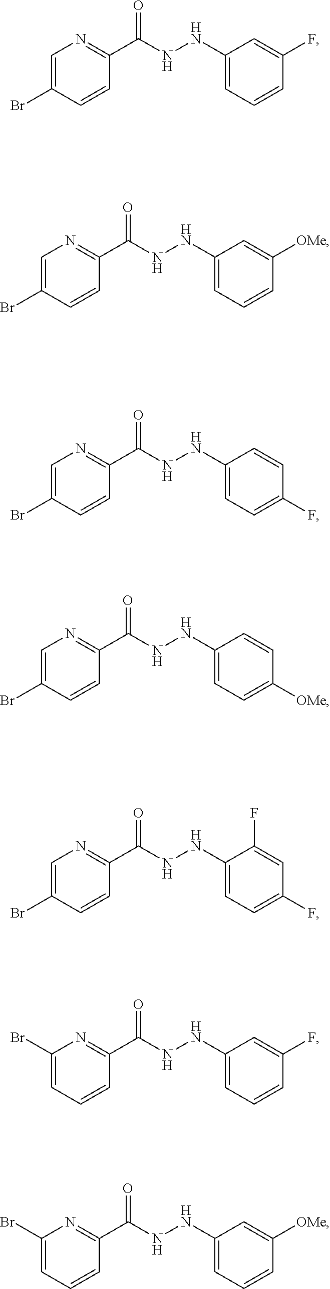

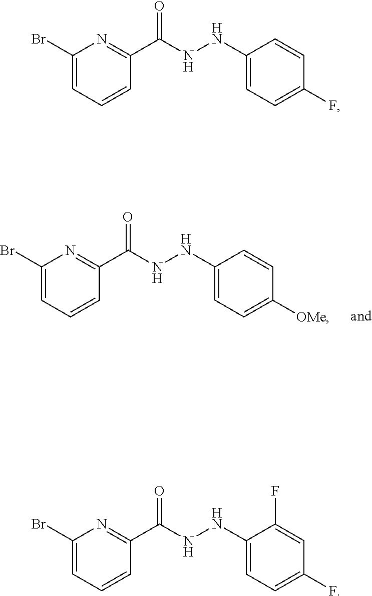 [SMILES: O=C(NNc1ccc(F)cc1)c1cccc(Br)n1 ; COc1ccc(NNC(=O)c2cccc(Br)n2)cc1 ; O=C(NNc1ccc(F)cc1F)c1cccc(Br)n1]

In some embodiments, the compound has the formula selected from the group consisting of:

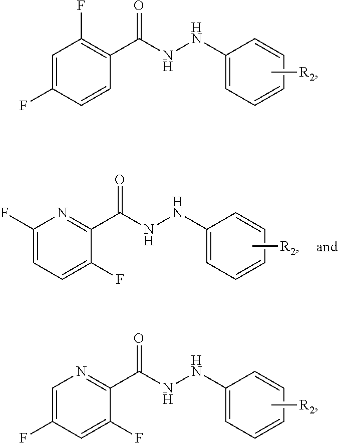

in which R₂ is selected from the group consisting of 3-OMe, 4-OMe, 3-Cl, 4-Cl, 3,5-diCl, 3-F, 4-F, 2,4-diF, 2,5-diF, and 3,5-diF.

In some embodiments, the compound has the formula selected from the group

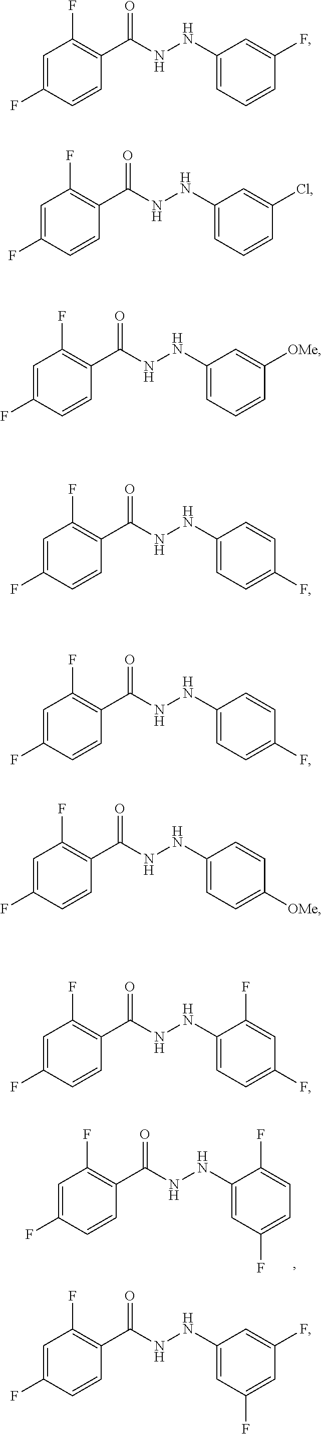

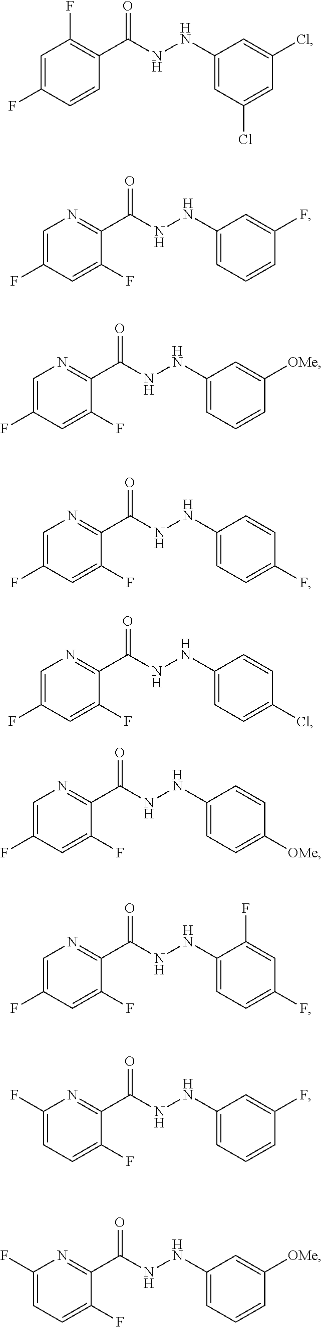

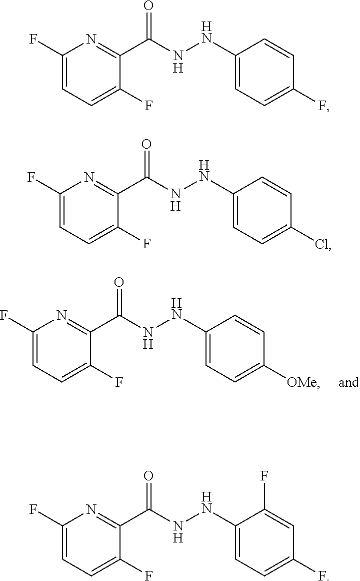

The presently-disclosed subject matter further includes methods for synthesizing compounds useful as antifungal agents.

The presently-disclosed subject matter further includes compositions including a compound as disclosed herein and a suitable pharmaceutical carrier. In some embodiments, the composition includes at least two compounds as disclosed herein.

The presently-disclosed subject matter further includes methods of treating a fungal infection in a subject, comprising administering to the subject an effective amount of a compound or composition as disclosed herein. In some embodiments, the subject is a human subject. In some embodiments, the subject is a plant or crop.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “administering” refers to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In some embodiments, oral administration is used. In some embodiments, intravenous (IV) administration is used.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

As used herein, the terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

As used herein, the terms “treatment” or “treating” refer to the medical management of a subject with the intent to cure, ameliorate, reduce, or prevent or slow progression of a fungal infection.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Synthesis

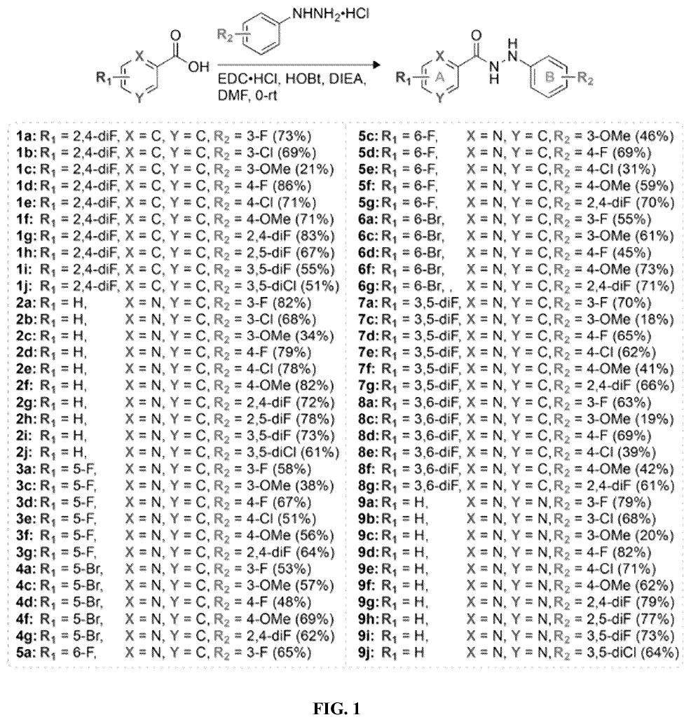

For the studies described in these examples, 64 monohydrazides were synthesized with different R₁ and R₂ substituents (FIG. 1). The coupling reaction between various commercially available acids and commercially available substituted hydrazines using coupling reagents (N-(3-dimethylaminopropyl)-N′-ethylcarbodiimide hydrochloride and 1-hydroxy-benzotriazole hydrate) resulted in the formation of compounds 1a-9j in 18-86% yields. The entire library of monohydrazides was separated into nine different series (1-9) based on the R₁ and R₂ substituents (denoted by a-j in compound numbering) on rings A and B, respectively.

### Example 2: Materials and Instrumentation

The chemicals used in this study were purchased from Sigma-Aldrich (St. Louis, MO), AK Scientific (Union City, CA), Acros Organics (New Jersey, NJ), TCI America (Portland, OR), Oakwood Chemicals (Estill, SC), Combi-Blocks (San Diego, CA), Accela Chembio (San Diego, CA), and Chem-Impex (Wood Dale, IL), and used without any further purification. Chemical reactions were monitored by thin layer chromatography (TLC) (Merck, silica gel 60 F₂₅₄) and visualized using UV light. Compounds were purified by SiO₂ flash chromatography (Dynamic Adsorbents Inc., flash SiO₂ gel 32-63p).¹H and ¹³C NMR spectra were recorded on Agilent VNMRS-500, MR-400, or MR-600 (for both 1H and ¹³C) spectrometers using deuterated solvents, as specified. Chemical shifts (d) are given in parts per million (ppm). Coupling constants (J) are given in Hertz (Hz), and conventional abbreviations used for signal shape are as follows: br s; broad singlet; d, doublet; dd, doublet of doublets; ddd, doublet of doublet of doublets; dt, doublet of triplets; m, multiplet; s, singlet; t, triplet; td, triplet of doublets; tt, triplet of triplets. High resolution-mass spectrometry (HRMS) was carried out using a Shimadzu prominence LC system equipped with an AB SCIEX Triple TOFTM 5600 mass spectrometer (Shimadzu manufacturing, Kyoto, Japan). HRMS [M+H]⁺ signals were consistent with the expected molecular weights for all of the reported compounds. Further confirmation of purity for these final molecules was obtained by reversed-phase high-performance liquid chromatography (RP-HPLC) on an Agilent Technologies 1260 Infinity HPLC system by using the following general method: flow rate=0.5 mL/min; λ=254 nm; column=Vydac 201SP™ C18, 250×4.6 mm, 90 Å; 5 μm; eluents: A=H₂O+0.1% TFA, B=MeCN; gradient profile: starting from 5% B, increasing from 5% B to 100% B over 20 min, holding at 100% B for 7 min, decreasing from 100% B to 5% B in 3 min. Prior to each injection, the HPLC column was equilibrated for 15 min with 5% B. All compounds were at least 95% pure. Compounds 1a, 1e, 2a, 2b, 2c, 2e, 3a, 3c, 3d, 3e, 3f, 4a, 5a, 5e, 6a, 7a, 7e, 8a, 8e, 9a, and 9e were prepared and purified as previously reported.⁶⁴

### Example 3: Synthesis of Compound 1b (SGT1772)

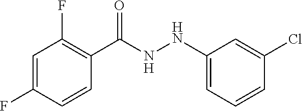 [SMILES: O=C(NNc1cccc(Cl)c1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 3-chlorophenylhydrazine hydrochloride (147 mg, 0.82 mmol). The reaction mixture was stirred at room temperature for 12 h, and the progress of the reaction was monitored by TLC (2:3/EtOAc:Hexanes, R/0.63). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 1:4/EtOAc:Hexanes) to afford compound 1b (122 mg, 69%) as a white solid: ¹H NMR (400 MHZ, (CD₃)₂SO) δ 10.26 (s, 1H), 8.32 (s, 1H), 7.74 (td, J₁=8.4 Hz, J₂=6.6 Hz, 1H), 7.43 (ddd, J₁=10.6 Hz, J₂=9.4 Hz, J₃=2.5 Hz, 1H), 7.26-7.20 (m, 1H), 7.19 (t, J=8.0 Hz, 1H), 6.79-6.72 (m, 3H); ¹³C NMR (150 MHZ, (CD₃)₂SO) δ 164.7 and 164.6 and 163.0 and 162.9 (dd, J₁=249.1 Hz, J₂=12.0 Hz), 163.5, 160.84 and 160.76 and 159.2 and 159.1 (dd, J₁=250.4 Hz, J₂=12.9 Hz), 150.7, 133.7, 131.99 and 131.96 and 131.92 and 131.89 (dd, J₁=10.1 Hz, J₂=4.3 Hz), 130.7, 119.44 and 119.42 and 119.34 and 119.32 (dd, J₁=15.7 Hz, J₂=3.2 Hz), 118.4, 112.33 and 112.31 and 112.19 and 112.17 (dd, J₁=21.6 Hz, J₂=2.0 Hz), 111.7, 111.0, 105.1 and 104.9 and 104.7 (t, J=26.9 Hz); HRMS m/z calcd for C₁₃H₉ClF₂N₂O [M+H]⁺: 283.0449; found 283.0446. The purity of the compound was further confirmed by HPLC: Rₜ=16.15 min (99% pure).

### Example 4: Synthesis of Compound 1c (SGT1771)

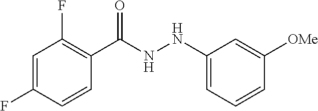 [SMILES: COc1cccc(NNC(=O)c2ccc(F)cc2F)c1]

To a solution of 2,4-difluorobenzoic acid (125 mg, 0.79 mmol) in DMF (3 mL) at 0° C., N-(3-dimethylaminopropyl)-N′-ethylcarbodiimide hydrochloride (182 mg, 0.95 mmol), 1-hydroxybenzotriazole hydrate (128 mg, 0.95 mmol), and N,N-diisopropylethyl amine (0.41 mL, 2.37 mmol) were added. The reaction mixture was stirred at 0° C. for 30 min followed by the addition of 3-methoxyphenylhydrazine hydrochloride (166 mg, 0.85 mmol). The reaction mixture was stirred at room temperature for 12 h, and the progress of the reaction was monitored by TLC (2:3/EtOAc:Hexanes, R/0.68). The reaction was quenched with H₂O (100 mL) and extracted with EtOAc (70 mL). The organic layer was washed with H₂O (70 mL), brine (20 mL), dried over Na₂SO₄, and concentrated under reduced pressure. The resulting residue was purified by flash column chromatography (SiO₂, 2:3/EtOAc:Hexanes) to afford compound 1c (46 mg, 21%) as a pale yellow solid: ¹H NMR (500 MHz, CD₃OD) d 7.84-7.80 (m, 1H), 7.17-7.08 (m, 3H), 6.51-6.47 (m, 2H), 6.41 (ddd, J₁=8.2 Hz, J₂=2.5 Hz, J₃=0.9 Hz, 1H), 3.75 (s, 3H); ¹³C NMR (150 MHz, (CD₃)₂SO) δ 164.4 and 164.3 and 162.7 and 162.6 (dd, J₁=248.2 Hz, J₂=12.0 Hz), 163.2, 160.65 and 160.56 and 159.0 and 158.9 (dd, J₁=250.2 Hz, J₂=13.0 Hz), 160.2, 150.5, 131.74 and 131.71 and 131.67 and 131.64 (dd, J₁=10.6 Hz, J₂=5.1 Hz), 129.6, 119.68 and 119.66 and 119.58 and 119.56 (dd, J₁=15.2 Hz, J₂=3.3 Hz), 112.09 and 112.06 and 111.94 and 111.92 (dd, J₁=21.6 Hz, J₂=3.9 Hz), 105.1 and 104.2 (d, J=138.6 Hz), 104.9 and 104.7 and 104.5 (t, J=26.0 Hz), 98.1, 54.7; HRMS m/z calcd for C₁₄H₁₂F₂N₂O₂ [M+H]⁺: 279.0945; found 279.0935. The purity of the compound was further confirmed by HPLC: Rₜ=15.40 min (95% pure).

### Example 5: Synthesis of Compound 1d (SGT1785)

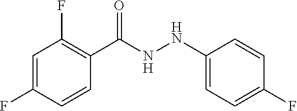 [SMILES: O=C(NNc1ccc(F)cc1)c1ccc(F)cc1F]

To a solution of 2,4 difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol) and N,N-diisopropylethyl amine (0.33 mL, 1.89 mmol) were added. The reaction mixture was stirred at 0° C. for 30 min followed by the addition of 4-fluorophenylhydrazine hydrochloride (134 mg, 0.82 mmol). The reaction mixture was stirred at room temperature for 12 h, and the progress of the reaction was monitored by TLC (2:3/EtOAc:Hexanes, R/0.43). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:7/EtOAc:Hexanes) to afford compound 1d (144 mg, 86%) as a white solid: ¹H NMR (500 MHZ, (CD₃)₂SO) δ 10.21 (d, J=2.9 Hz, 1H), 7.99 (d, J=2.9 Hz, 1H), 7.73 (td, J₁=8.5 Hz, J₂=6.6 Hz, 1H), 7.41 (ddd, J₁=10.6 Hz, J₂=9.5 Hz, J₃=2.5 Hz, 1H), 7.24-7.19 (m, 1H), 7.02 (t, J=8.9 Hz, 2H), 6.80 (dd, J₁=9.1 Hz, J₂=4.7 Hz, 2H); ¹³C NMR (150 MHz, (CD₃)₂SO) d 164.4 and 164.3 and 162.8 and 162.7 (dd, J₁=248.2 Hz, J₂=12.0 Hz), 163.2, 160.7 and 160.6 and 159.0 and 158.9 (dd, J₁=250.9 Hz, J₂=13.0 Hz), 156.7 and 155.2 (d, J=231.8 Hz), 145.6, 131.80 and 131.77 and 131.73 and 131.70 (dd, J₁=10.0 Hz, J₂=4.4 Hz), 119.56 and 119.53 and 119.5 and 119.4 (dd, J₁=15.3 Hz, J₂=4.2 Hz), 115.3 and 115.2 (d, J=21.8 Hz), 113.5 and 113.4 (d, J=7.8 Hz), 112.1 and 112.0 and 111.92 and 111.89 (dd, J₁=21.6 Hz, J₂=4.1 Hz), 104.9 and 104.7 and 104.6 (t, J=26.5 Hz); HRMS m/z calcd for C₁₃H₉F₃N₂O [M+H]⁺: 267.0745; found 267.0741. The purity of the compound was further confirmed by HPLC: Rₜ=15.63 min (98% pure).

### Example 6: Synthesis of Compound 1f (SGT1771)

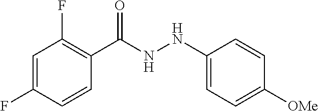 [SMILES: COc1ccc(NNC(=O)c2ccc(F)cc2F)cc1]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-methoxyphenylhydrazine hydrochloride (143 mg, 0.82 mmol). The reaction mixture was stirred at room temperature for 12 h, and the progress of the reaction was monitored by TLC (2:3/EtOAc:Hexanes, R/0.65). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 1:4/EtOAc:Hexanes) to afford compound 1f (124 mg, 71%) as a yellow solid: ¹H NMR (500 MHz, (CD₃)₂SO) d 10.16 (d, J=3.5 Hz, 1H), 7.71 (td, J₁=8.4 Hz, J₂=6.7 Hz, 1H), 7.68 (d, J=3.3 Hz, 1H), 7.40 (ddd, J₁=10.6 Hz, J₂=9.5 Hz, J₃=2.5 Hz, 1H), 7.24-7.18 (m, 1H), 6.80 (d, J=9.4 Hz, 2H), 6.77 (d, J=9.3 Hz, 2H); ¹³C NMR (150 MHZ, (CD₃)₂SO) δ 164.35 and 164.27 and 162.7 and 162.6 (dd, J₁=248.2 Hz, J₂=12.0 Hz), 163.2, 160.66 and 160.58 and 159.0 and 158.9 (dd, J₁=250.2 Hz, J₂=12.6 Hz), 152.8, 142.9, 131.8 and 131.73 and 131.69 and 131.66 (dd, J₁=10.0 Hz, J₂=4.4 Hz), 119.74 and 119.72 and 119.64 and 119.62 (dd, J₁=15.0 Hz, J₂=3.1 Hz), 114.3, 113.8, 112.03 and 111.99 and 111.88 and 111.86 (dd, J₁=21.5 Hz, J₂=3.9 Hz), 104.9 and 104.7 and 104.5 (t, J=26.0 Hz), 55.3; HRMS m/z calcd for C₁₄H₁₂F₂N₂O₂ [M+H]⁺: 279.0945; found 279.0947. The purity of the compound was further confirmed by HPLC: Rₜ=15.29 min (99% pure).

### Example 7: Synthesis of Compound 1g (SGT1393)

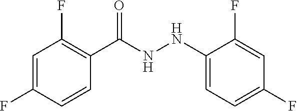 [SMILES: O=C(NNc1ccc(F)cc1F)c1ccc(F)cc1F]

To a solution of 2,4 difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 2,4-difluorophenylhydrazine hydrochloride (148 mg, 0.82 mmol). The reaction mixture was stirred at room temperature for 12 h, and the progress of the reaction was monitored by TLC (1:4/EtOAc:Hexanes, R/0.31). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 1:4/EtOAc:Hexanes) to afford compound 1g (149 mg, 83%) as a white solid: ¹H NMR (500 MHz, (CD₃)₂SO) δ 10.27 (s, 1H), 7.90 (s, 1H), 7.73 (td, J₁=8.4 Hz, J₂=6.6 Hz, 1H), 7.42 (ddd, J₁=10.6 Hz, J₂=9.4 Hz, J₃=2.5 Hz, 1H), 7.25-7.20 (m, 1H), 7.17 (ddd, J₁=11.7 Hz, J₂=8.9 Hz, J₃=2.8 Hz, 1H), 6.96-6.91 (m, 1H), 6.87 (td, J₁=9.4 Hz, J₂=5.8 Hz, 1H); ¹³C NMR (150 MHz, (CD₃)₂SO) δ 164.5 and 164.4 and 162.85 and 162.76 (dd, J₁=249.0 Hz, J₂=12.7 Hz), 163.33 and 163.32 (d, J=1.6 Hz), 160.75 and 160.66 and 159.1 and 159.0 (dd, J₁=251.2 Hz, J₂=12.9 Hz), 155.7 and 155.6 and 154.13 and 154.06 (dd, J₁=235.1 Hz, J₂=10.8 Hz), 150.43 and 150.35 and 148.8 and 148.7 (dd, J₁=241.5 Hz, J₂=12.0 Hz), 133.42 and 133.41 and 133.36 and 133.34 (dd, J₁=10.9 Hz, J₂=2.9 Hz), 131.9 and 131.82 and 131.78 and 131.75 (dd, J₁=10.1 Hz, J₂=4.6 Hz), 119.32 and 119.29 and 119.22 and 119.19 (dd, J₁=15.2 Hz, J₂=4.1 Hz), 114.18 and 114.15 and 114.12 and 114.09 (dd, J₁=8.8 Hz, J₂=4.4 Hz), 112.09 and 112.07 and 111.95 and 111.92 (dd, J₁=21.0 Hz, J₂=3.3 Hz), 110.98 and 110.96 and 110.84 and 110.81 (dd, J₁=21.6 Hz, J₂=3.7 Hz), 104.9 and 104.8 and 104.6 (t, J=26.0 Hz), 104.0 and 103.80 and 103.77 and 103.6 (dd, J₁=27.0 Hz, J₂=22.6 Hz); HRMS m/z calcd for C₁₃H₈F₄N₂O [M+H]⁺: 285.0651; found 285.0632. The purity of the compound was further confirmed by HPLC: Rₜ=16.02 min (99% pure).

### Example 8: Synthesis of Compound 1h (SGT1769)

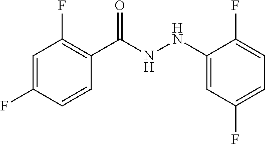 [SMILES: O=C(NNc1cc(F)ccc1F)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 2,5-difluorophenylhydrazine hydrochloride (148 mg, 0.82 mmol). The reaction mixture was stirred at room temperature for 12 h, and the progress of the reaction was monitored by TLC (2:3/EtOAc:Hexanes, R/0.61). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 1:4/EtOAc:Hexanes) to afford compound 1h (119 mg, 67%) as a white solid: ¹H NMR (500 MHZ, (CD₃)₂SO) δ 10.30 (s, 1H), 8.29 (s, 1H), 7.76 (td, J₁=8.5 Hz, J₂=6.6 Hz, 1H), 7.43 (ddd, J₁=10.7 Hz, J₂=9.6 Hz, J₃=2.5 Hz, 1H), 7.26-7.20 (m, 1H), 7.14 (ddd, J₁=11.4 Hz, J₂=8.9 Hz, J₃=5.1 Hz, 1H), 6.59 (ddd, J₁=10.2 Hz, J₂=6.9 Hz, J₃=3.1 Hz, 1H), 6.53 (tt, J₁=8.3 Hz, J₂=3.3 Hz, 1H); ¹³C NMR (150 MHz, (CD₃)₂SO) δ 164.6 and 164.5 and 162.9 and 162.8 (dd, J₁=249.1 Hz, J₂=12.5 Hz), 163.3, 160.8 and 160.7 and 159.1 and 159.0 (dd, J₁=250.3 Hz, J₂=12.9 Hz), 159.8 and 158.2 (d, J=235.9 Hz), 146.98 and 146.97 and 145.42 and 145.41 (dd, J₁=233.9 Hz, J₂=1.3 Hz), 138.3 and 138.20 and 138.19 and 138.1 (dd, J₁=12.6 Hz, J₂=11.1 Hz), 131.90 and 131.87 and 131.83 and 131.80 (dd, J₁=10.6 Hz, J₂=5.0 Hz), 119.20 and 119.18 and 119.10 and 119.08 (dd, J₁=15.0 Hz, J₂=3.3 Hz), 116.0 and 115.94 and 115.88 and 115.8 (dd, J₁=20.4 Hz, J₂=10.5 Hz), 112.15 and 112.12 and 112.0 and 111.98 (dd, J₁=21.6 Hz, J₂=3.8 Hz), 105.0 and 104.8 and 104.6 (t, J=26.1 Hz), 104.1 and 104.0 and 103.9 and 103.8 (dd, J₁=24.0 Hz, J₂=7.3 Hz), 100.19 and 100.16 and 99.99 and 99.97 (dd, J₁=29.1 Hz, J₂=3.9 Hz); HRMS m/z calcd for C₁₃H₈F₄N₂O [M+H]⁺: 285.0651; found 285.0654. The purity of the compound was further confirmed by HPLC: Rₜ=15.86 min (100% pure).

### Example 9: Synthesis of Compound 1i (SGT1770)

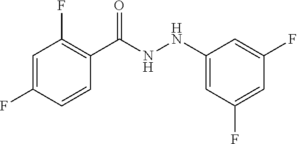 [SMILES: O=C(NNc1cc(F)cc(F)c1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 3,5-difluorophenylhydrazine hydrochloride (148 mg, 0.82 mmol). The reaction mixture was stirred at room temperature for 12 h, and the progress of the reaction was monitored by TLC (2:3/EtOAc:Hexanes, R/0.64). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 1:4/EtOAc:Hexanes) to afford compound 1i (99 mg, 55%) as a white solid: ¹H NMR (500 MHZ, (CD₃)₂SO) δ 10.32 (s, 1H), 8.61 (s, 1H), 7.77 (td, J₁=8.4 Hz, J₂=6.5 Hz, 1H), 7.43 (ddd, J₁=11.8 Hz, J₂=9.4 Hz, J₃=2.5 Hz, 1H), 7.25-7.20 (m, 1H), 6.48 (tt, J₁=9.4 Hz, J₂=2.3 Hz, 1H), 6.39 (dd, J₁=10.0 Hz, J₂=2.3 Hz, 2H); ¹³C NMR (150 MHz, (CD₃)₂SO) δ 164.6 and 164.5 and 162.9 and 162.8 (dd, J₁=249.0 Hz, J₂=12.1 Hz), 164.2 and 164.1 and 162.6 and 162.5 (dd, J₁=241.2 Hz, J₂=16.1 Hz), 163.3, 160.7 and 160.6 and 159.1 and 159.0 (dd, J₁=250.3 Hz, J₂=12.6 Hz), 152.2 and 152.14 and 152.06 (t, J=12.8 Hz), 131.93 and 131.90 and 131.86 and 131.83 (dd, J₁=10.6 Hz, J₂=4.3 Hz), 119.18 and 119.15 and 119.08 and 119.05 (dd, J₁=15.2 Hz, J₂=4.0 Hz), 112.2 and 112.1 and 112.02 and 111.99 (dd, J₁=21.5 Hz, J₂=3.6 Hz), 105.0 and 104.8 and 104.6 (t, J=26.0 Hz), 95.0 and 94.8 (d, J=29.0 Hz), 93.4 and 93.2 and 93.1 (t, J=26.1 Hz); HRMS m/z calcd for C₁₃H₈F₄N₂O [M+H]⁺: 285.0651; found 285.0622. The purity of the compound was further confirmed by HPLC: Rₜ=15.92 min (99% pure).

### Example 10: Synthesis of Compound 1j (SGT1773)

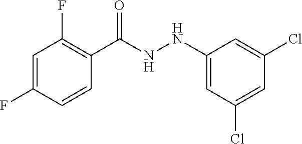 [SMILES: O=C(NNc1cc(Cl)cc(Cl)c1)c1ccc(F)cc1F]

To a solution of 2,4-difluorobenzoic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 3,5-dichlorophenylhydrazine hydrochloride (175 mg, 0.82 mmol). The reaction mixture was stirred at room temperature for 12 h, and the progress of the reaction was monitored by TLC (2:3/EtOAc:Hexanes, R/0.71). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 1:4/EtOAc:Hexanes) to afford compound 1j (102 mg, 51%) as a yellow solid: ¹H NMR (400 MHZ, (CD₃)₂SO) δ 10.33 (s, 1H), 8.61 (s, 1H), 7.76 (td, J₁=8.4 Hz, J₂=6.6 Hz, 1H), 7.44 (ddd, J₁=10.7 Hz, J₂=9.4 Hz, J₃=2.5 Hz, 1H), 7.277.20 (m, 1H), 6.87 (t, J=1.8 Hz, 1H), 6.75 (d, J=1.8 Hz, 2H); ¹³C NMR (150 MHZ, (CD₃)₂SO) δ 164.6 and 164.5 and 162.9 and 162.8 (dd, J₁=249.7 Hz, J₂=12.8 Hz), 163.2, 160.7 and 160.6 and 159.1 and 159.0 (dd, J₁=250.2 Hz, J₂=11.9 Hz), 151.5, 134.5, 131.91 and 131.88 and 131.84 and 131.81 (dd, J₁=10.5 Hz, J₂=4.7 Hz), 119.1 and 119.0 and 118.96 and 118.93 (dd, J₁=14.9 Hz, J₂=2.9 Hz), 117.5, 112.22 and 112.20 and 112.08 and 112.06 (dd, J₁=22.0 Hz, J₂=2.9 Hz), 110.3, 105.0 and 104.8 and 104.6 (t, J=26.1 Hz); HRMS m/z calcd for C₁₃H₈Cl₂F₂N₂O [M+H]⁺: 317.0060; found 317.0052. The purity of the compound was further confirmed by HPLC: Rₜ=17.08 min (99% pure).

### Example 11: Synthesis of Compound 2d (SGT1776)

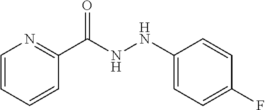 [SMILES: O=C(NNc1ccc(F)cc1)c1ccccn1]

To a solution of 2-picolinic acid (100 mg, 0.81 mmol) in DMF (2 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 4-fluorophenylhydrazine hydrochloride (172 mg, 1.06 mmol). The reaction mixture was stirred at room temperature for 12 h, and the progress of the reaction was monitored by TLC (2:3/EtOAc:Hexanes, R/0.41). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2d (148 mg, 79%) as a pale yellow solid: 1H NMR (500 MHZ, (CD₃)₂SO) δ 10.58 (d, J=3.3 Hz, 1H), 8.70-8.68 (m, 1H), 8.04-7.98 (m, 2H), 7.87 (d, J=3.4 Hz, 1H), 7.64 (ddd, J₁=8.3 Hz, J₂=4.8 Hz, J₃=3.3 Hz, 1H), 6.98 (t, J=8.9 Hz, 2H), 6.76 (dd, J₁=9.1 Hz, J₂=4.7 Hz, 2H); ¹³C NMR (100 MHZ, (CD₃)₂SO) δ 164.0, 157.1 and 154.7 (d, J=232.1 Hz), 149.7, 148.6, 145.79 and 145.78 (d, J=1.8 Hz), 137.8, 126.8, 122.2, 115.2 and 115.0 (d, J=22.3 Hz), 113.6 and 113.5 (d, J=7.6 Hz); HRMS m/z calcd for C₁₂H₁₀FN₃O [M+H]⁺: 232.0886; found 232.0856. The purity of the compound was further confirmed by HPLC: Rₜ=15.21 min (96% pure).

### Example 12: Synthesis of Compound 2f (SGT1778)

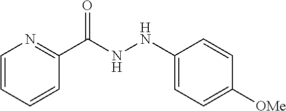 [SMILES: COc1ccc(NNC(=O)c2ccccn2)cc1]

To a solution of 2-picolinic acid (100 mg, 0.81 mmol) in DMF (2 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 4-methoxyphenylhydrazine hydrochloride (185 mg, 1.06 mmol). The reaction mixture was stirred at room temperature for 12 h, and the progress of the reaction was monitored by TLC (2:3/EtOAc:Hexanes, R/0.43). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2f (162 mg, 82%) as an yellow solid: ¹H NMR (500 MHZ, (CD₃)₂SO) δ 10.50 (d, J=3.8 Hz, 1H), 8.69-8.67 (m, 1H), 8.03-7.98 (m, 2H), 7.63 (ddd, J₁=8.0 Hz, J₂=4.8 Hz, J₃=3.1 Hz, 1H), 7.57 (d, J=3.8 Hz, 1H), 6.78-6.72 (m, 4H), 3.33 (s, 3H); ¹³C NMR (150 MHz, (CD₃)₂SO) δ 163.9, 152.7, 149.8, 148.6, 143.1, 137.8, 126.7, 122.2, 114.2, 113.9, 55.3; HRMS m/z calcd for C₁₃H₁₃N₃O₂ [M+H]⁺: 244.1086; found 244.1056. The purity of the compound was further confirmed by HPLC: Rₜ=14.67 min (96% pure).

### Example 13: Synthesis of Compound 2g (SGT1396)

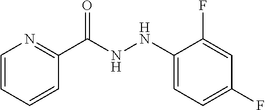 [SMILES: O=C(NNc1ccc(F)cc1F)c1ccccn1]

To a solution of 2-picolinic acid (100 F mg, 0.81 mmol) in DMF (3 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 2,4-difluorophenylhydrazine hydrochloride (191 mg, 1.06 mmol). The reaction mixture was stirred at room temperature for 12 h, and the progress of the reaction was monitored by TLC (2:3/EtOAc:Hexanes, R/0.38). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2g (145 mg, 72%) as a yellow solid: ¹H NMR (500 MHZ, (CD₃)₂SO) δ 10.64 (d, J=2.8 Hz, 1H), 8.69 (dt, J₁=4.8 Hz, J₂=1.3 Hz, 1H), 8.04-8.00 (m, 2H), 7.77 (very br s, 1H), 7.65 (ddd, J₁=6.0 Hz, J₂=4.8 Hz, J₃=2.9 Hz, 1H), 7.16 (ddd, J₁=11.8 Hz, J₂=8.9 Hz, J₃=2.8 Hz, 1H), 6.90-6.84 (m, 1H), 6.78 (td, J₁=9.5 Hz, J₂=5.8 Hz, 1H); ¹³C NMR (100 MHZ, (CD₃)₂SO) δ 164.1, 156.0 and 155.9 and 153.6 and 153.5 (dd, J₁=235.5 Hz, J₂=10.8 Hz), 150.9 and 150.8 and 148.5 and 148.3 (dd, J₁=241.4 Hz, J₂=12.1 Hz), 149.5, 148.7, 137.8, 133.7 and 133.65 and 133.57 and 133.5 (dd, J₁=13.8 Hz, J₂=3.0 Hz), 126.9, 122.3, 114.35 and 114.30 and 114.26 and 114.21 (dd, J₁=9.1 Hz, J₂=4.7 Hz), 110.91 and 110.88 and 110.69 and 110.66 (dd, J₁=21.7 Hz, J₂=3.5 Hz), 103.9 and 103.7 and 103.6 and 103.4 (dd, J₁=26.7 Hz, J₂=22.3 Hz); HRMS m/z calcd for C₁₂H₉F₂N₃O [M+H]⁺: 250.0792; found 250.0781. The purity of the compound was further confirmed by HPLC: Rₜ=15.36 min (95% pure).

### Example 14: Synthesis of Compound 2h (SGT1789)

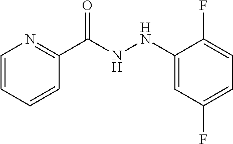 [SMILES: O=C(NNc1cc(F)ccc1F)c1ccccn1]

To a solution of 2-picolinic acid (100 mg, 0.81 mmol) in DMF (3 mL) at 0° C., N-(3-dimethylaminopropyl)-N′-ethylcarbodiimide hydrochloride (203 mg, 1.06 mmol) and N,N-diisopropylethyl amine (0.40 mL, 2.43 mmol) were added. The reaction mixture was stirred at 0° C. for 30 min followed by the addition of 2,5-difluorophenylhydrazine hydrochloride (191 mg, 1.06 mmol). The reaction mixture was stirred at room temperature for 12 h, and the progress of the reaction was monitored by TLC (3:2/EtOAc:Hexanes, R/0.32). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 2h (157 mg, 78%) as a pale yellow solid: ¹H NMR (500 MHZ, (CD₃)₂SO) δ 10.67 (br s, 1H), 8.70 (dt, J₁=4.7 Hz, J₂=1.5 Hz, 1H), 8.17 (br s, 1H), 8.04-8.01 (m, 2H), 7.69-7.64 (m, 1H), 7.16-7.10 (m, 1H), 6.52-6.44 (m, 2H); ¹³C NMR (150 MHz, (CD₃)₂SO) δ 164.1, 159.7 and 158.1 (d, J=235.8 Hz), 149.5, 148.7, 147.0 and 145.5 (d, J=233.4 Hz), 138.6 and 138.5 and 138.4 (t, J=11.9 Hz), 137.8, 127.0, 122.4, 115.9 and 115.8 and 115.72 and 115.66 (dd, J₁=20.5 Hz, J₂=10.5 Hz), 103.8 and 103.7 and 103.6 and 103.5 (dd, J₁=24.2 Hz, J₂=7.2 Hz), 100.21 and 100.19 and 100.02 and 100.00 (dd, J₁=32.6 Hz, J₂=3.4 Hz); HRMS m/z calcd for C₁₂H₉F₂N₃O [M+H]⁺: 250.0792; found 250.0768. The purity of the compound was further confirmed by HPLC: Rₜ=15.54 min (98% pure).

### Example 15: Synthesis of Compound 2i (SGT1790)

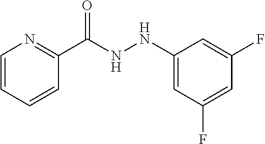 [SMILES: O=C(NNc1cc(F)cc(F)c1)c1ccccn1]

To a solution of 2-picolinic acid (100 mg, 0.81 mmol) in DMF (3 mL) at 0° C., N-(3-dimethylaminopropyl)-N′-ethylcarbodiimide hydrochloride (203 mg, 1.06 mmol) and N,N-diisopropylethyl amine (0.40 mL, 2.43 mmol) were added. The reaction mixture was stirred at 0° C. for 30 min followed by the addition of 3,5-difluorophenylhydrazine hydrochloride (191 mg, 1.06 mmol). The reaction mixture was stirred at room temperature for 12 h, and the progress of the reaction was monitored by TLC (3:2/EtOAc:Hexanes, R/0.33). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 2i (147 mg, 73%) as a pale yellow solid: ¹H NMR (500 MHZ, (CD₃)₂SO) δ 10.72 (s, 1H), 8.70 (dt, J₁=4.8 Hz, J₂=1.4 Hz, 1H), 8.51 (br s, 1H), 8.04-8.01 (m, 2H), 7.66 (sextet, J=4.6 Hz, 1H), 6.44 (ddd, J₁=11.6 Hz, J₂=4.5 Hz, J₃=2.2 Hz, 1H), 6.36-6.31 (m, 2H); ¹³C NMR (150 MHz, (CD₃)₂SO) δ 164.09 and 164.0 and 162.5 and 162.4 (dd, J₁=240.5 Hz, J₂=16.1 Hz), 164.1, 152.5 and 152.4 and 152.3 (t, J=13.0 Hz), 149.4 and 148.7 (d, J=106.0 Hz), 137.8, 127.0, 122.4, 94.9 and 94.7 (d, J=29.1 Hz), 94.88 and 94.77 (d, J=16.4 Hz), 93.1 and 92.9 and 92.7 (t, J=26.1 Hz); HRMS m/z calcd for C₁₂H₉F₂N₃O [M+H]⁺: 250.0792; found 250.0770. The purity of the compound was further confirmed by HPLC: Rₜ=15.48 min (98% pure).

### Example 16: Synthesis of Compound 2j (SGT1788)

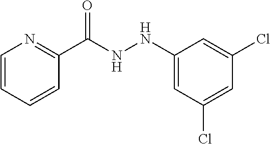 [SMILES: O=C(NNc1cc(Cl)cc(Cl)c1)c1ccccn1]

To a solution of 2-picolinic acid (100 mg, 0.81 mmol) in DMF (3 mL) at 0° C., N-(3-dimethylaminopropyl)-N′-ethylcarbodiimide hydrochloride (203 mg, 1.06 mmol), 1-hydroxybenzotriazole hydrate (143 mg, 1.06 mmol), and N,N-diisopropylethyl amine (0.40 mL, 2.43 mmol) were added. The reaction mixture was stirred at 0° C. for 30 min followed by the addition of 3,5-dichlorophenylhydrazine hydrochloride (226 mg, 1.06 mmol). The reaction mixture was stirred at room temperature for 12 h, and the progress of the reaction was monitored by TLC (2:3/EtOAc:Hexanes, R_{f}0.37). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 2:3/EtOAc:Hexanes) to afford compound 2j (139 mg, 61%) as a yellow solid: ¹H NMR (500 MHZ, (CD₃)₂SO) § 10.76 (br s, 1H), 8.70 (dt, J₁=4.7 Hz, J₂=1.4 Hz, 1H), 8.51 (d, J=2.1 Hz, 1H), 8.04-8.02 (m, 2H), 7.67 (sextet, J=4.6 Hz, 1H), 6.83 (t, J=1.9 Hz, 1H), 6.69 (d, J=1.9 Hz, 2H); ¹³C NMR (150 MHZ, (CD₃)₂SO) δ 164.0, 151.7, 149.3, 148.7, 137.9, 134.3, 127.1, 122.4, 117.1, 110.3; HRMS m/z calcd for C₁₂H₉Cl₂N₃O [M+H]⁺: 282.0201; found 282.0168. The purity of the compound was further confirmed by HPLC: Rₜ=16.82 min (99% pure).

### Example 17: Synthesis of Compound 3g (SGT1803)

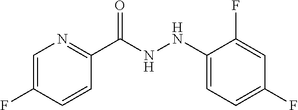 [SMILES: O=C(NNc1ccc(F)cc1F)c1ccc(F)cn1]

To a solution of 5-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 2,4-difluorophenylhydrazine hydrochloride (166 mg, 0.92 mmol). The reaction mixture was stirred at room temperature for 12 h, and the progress of the reaction was monitored by TLC (2:3/EtOAc:Hexanes, R/0.47). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 3g (122 mg, 64%) as a pale yellow solid: ¹H NMR (500 MHZ, (CD₃)₂SO) δ 10.64 (s, 1H), 8.70 (d, J=2.9 Hz, 1H), 8.10 (dd, J₁=8.5 Hz, J₂=4.7 Hz, 1H), 7.93 (td, J₁=8.8 Hz, J₂=2.9 Hz, 1H), 7.78 (s, 1H), 7.16 (ddd, J₁=11.8 Hz, J₂=8.9 Hz, J₃=2.8 Hz, 1H), 6.90-6.84 (m, 1H), 6.77 (td, J₁=9.5 Hz, J₂=5.8 Hz, 1H); ¹³C NMR (150 MHz, (CD₃)₂SO) δ 163.1, 161.7 and 160.0 (d, J=257.5 Hz), 155.6 and 155.5 and 154.02 and 153.94 (dd, J₁=235.0 Hz, J₂=10.6 Hz), 150.4 and 150.3 and 148.8 and 148.7 (dd, J₁=240.6 Hz, J₂=12.1 Hz), 146.3 and 146.2 (d, J=4.2 Hz), 137.3 and 137.0 (d, J=24.9 Hz), 133.60 and 133.58 and 133.53 and 133.50 (dd, J₁=10.8 Hz, J₂=3.1 Hz), 124.59, 124.55 and 124.46 (d, J=12.1 Hz), 114.30 and 114.27 and 114.24 and 114.21 (dd, J₁=9.5 Hz, J₂=4.6 Hz), 110.87 and 110.85 and 110.72 and 110.70 (dd, J₁=21.7 Hz, J₂=3.3 Hz), 103.8 and 103.7 and 103.6 and 103.5 (dd, J₁=26.6 Hz, J₂=22.0 Hz); HRMS m/z calcd for C₁₂H₈F₃N₃O [M+H]⁺: 268.0697; found 268.0702. The purity of the compound was further confirmed by HPLC: Rₜ=15.63 min (96% pure).

### Example 18: Synthesis of Compound 4c (SGT1436)

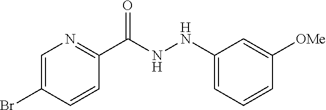 [SMILES: COc1cccc(NNC(=O)c2ccc(Br)cn2)c1]

To a solution of 5-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 3-methoxyphenylhydrazine hydrochloride (112 mg, 0.64 mmol). The reaction mixture was stirred at room temperature for 12 h, and the progress of the reaction was monitored by TLC (2:3/EtOAc:Hexanes, R/0.39). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 4c (92 mg, 57%) as a pale yellow solid: ¹H NMR (500 MHZ, (CD₃)₂SO) δ 10.60 (d, J=2.9 Hz, 1H), 8.82 (dd, J₁=2.4 Hz, J₂=0.7 Hz, 1H), 8.28 (dd, J₁=8.4 Hz, J₂=2.4 Hz, 1H), 7.95 (dd, J₁=8.3 Hz, J₂=0.6 Hz, 1H), 7.92 (d, J=2.8 Hz, 1H), 7.03 (dd, J₁=8.7 Hz, J₂=8.0 Hz, 1H), 6.35 (ddd, J₁=8.1 Hz, J₂=2.0 Hz, J₃=1.0 Hz, 1H), 6.32-6.28 (m, 2H), 3.66 (s, 3H); ¹³C NMR (150 MHz, (CD₃)₂SO) δ 163.3, 160.1, 150.6, 149.5, 148.5, 140.5, 129.5, 124.1, 123.7, 105.1, 103.9, 98.3, 54.8; HRMS m/z calcd for C₁₃H₁₂BrN₃O₂ [M+H]⁺: 322.0191; found 322.0177. The purity of the compound was further confirmed by HPLC: Rₜ=15.21 min (97% pure).

### Example 19: Synthesis of Compound 4d (SGT1774)

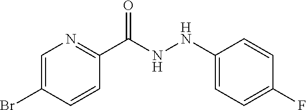 [SMILES: O=C(NNc1ccc(F)cc1)c1ccc(Br)cn1]

To a solution of 5-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (88 mg, 0.64 mmol), and N,N-diisopropylethyl amine (0.26 mL, 1.50 mmol) were added. The reaction mixture was stirred at 0° C. for 30 min followed by the addition of 4-fluorophenylhydrazine hydrochloride (104 mg, 0.64 mmol). The reaction mixture was stirred at room temperature for 12 h, and the progress of the reaction was monitored by TLC (2:3/EtOAc:Hexanes, R/0.67). The reaction was quenched with H₂O (80 mL) and extracted with EtOAc (70 mL). The organic layer was washed with H₂O (60 mL), brine (20 mL), dried over MgSO₄, and concentrated under reduced pressure. The resulting residue was purified by flash column chromatography (SiO₂, 3:7/EtOAc:Hexanes) to afford compound 4d (74 mg, 48%) as a white solid: ¹H NMR (400 MHZ, (CD₃)₂SO) δ 10.65 (d, J=3.2 Hz, 1H), 8.82 (dd, J₁=2.3 Hz, J₂=0.7 Hz, 1H), 8.27 (dd, J₁=8.4 Hz, J₂=2.3 Hz, 1H), 7.95 (dd, J₁=8.4 Hz, J₂=0.7 Hz, 1H), 7.88 (d, J=3.2 Hz, 1H), 6.98 (t, J=8.9 Hz, 2H), 6.75 (dd, J₁=9.0 Hz, J₂=4.7 Hz, 2H); ¹³C NMR (100 MHZ, (CD₃)₂SO) δ 163.4, 157.1 and 154.7 (d, J=232.3 Hz), 149.4, 148.5, 145.64 and 145.62 (d, J=1.7 Hz), 140.5, 124.1, 123.7, 115.2 and 115.0 (d, J=22.3 Hz), 113.6 and 113.5 (d, J=7.6 Hz); HRMS m/z calcd for C₁₂H₉BrFN₃O [M+H]⁺: 309.9991; found 309.9977. The purity of the compound was further confirmed by HPLC: Rₜ=16.23 min (95% pure).

### Example 20: Synthesis of Compound 4f (SGT1434)

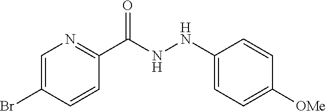 [SMILES: COc1ccc(NNC(=O)c2ccc(Br)cn2)cc1]

To a solution of 5-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 4-methoxyphenylhydrazine hydrochloride (112 mg, 0.64 mmol). The reaction mixture was stirred at room temperature for 12 h, and the progress of the reaction was monitored by TLC (2:3/EtOAc:Hexanes, R/0.38). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 4f (111 mg, 69%) as a pale yellow solid: ¹H NMR (500 MHz, (CD₃)₂SO) δ 10.59 (d, J=3.4 Hz, 1H), 8.81 (dd, J₁=2.3 Hz, J₂=0.7 Hz, 1H), 8.27 (dd, J₁=8.4 Hz, J₂=2.4 Hz, 1H), 7.94 (dd, J₁=8.4 Hz, J₂=0.7 Hz, 1H), 7.58 (d, J=3.5 Hz, 1H), 6.76 (d, J=9.1 Hz, 2H), 6.73 (d, J=9.3 Hz, 2H), 3.65 (s, 3H); ¹³C NMR (150 MHz, (CD₃)₂SO) δ 163.3, 152.8, 149.4, 148.6, 142.9, 140.4, 124.1, 123.6, 114.2, 113.9, 55.3; HRMS m/z calcd for C₁₃H₁₂BrN₃O₂ [M+H]⁺: 322.0191; found 322.0120. The purity of the compound was further confirmed by HPLC: Rₜ=16.01 min (99% pure).

### Example 21: Synthesis of Compound 4g (SGT1438)

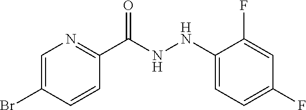 [SMILES: O=C(NNc1ccc(F)cc1F)c1ccc(Br)cn1]

To a solution of 5-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 2,4-difluorophenylhydrazine hydrochloride (116 mg, 0.64 mmol). The reaction mixture was stirred at room temperature for 12 h, and the progress of the reaction was monitored by TLC (2:3/EtOAc:Hexanes, R/0.47). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 4g (102 mg, 62%) as a white solid: ¹H NMR (500 MHZ, (CD₃)₂SO) δ 10.70 (s, 1H), 8.83 (dd, J₁=2.4 Hz, J₂=0.8 Hz, 1H), 8.28 (dd, J₁=8.5 Hz, J₂=2.4 Hz, 1H), 7.95 (dd, J₁=8.4 Hz, J₂=0.8 Hz, 1H), 7.79 (s, 1H), 7.16 (ddd, J₁=11.8 Hz, J₂=9.0 Hz, J₃=2.8 Hz, 1H), 6.90-6.84 (m, 1H), 6.77 (td, J₁=9.5 Hz, J₂=5.8 Hz, 1H); ¹³C NMR (150 MHz, (CD₃)₂SO) δ 163.4, 155.6 and 155.5 and 154.03 and 153.96 (dd, J₁=235.1 Hz, J₂=10.7 Hz), 150.43 and 150.35 and 148.8 and 148.7 (dd, J₁=241.4 Hz, J₂=12.6 Hz), 149.5, 148.3, 140.5, 133.51 and 133.49 and 133.43 and 133.42 (dd, J₁=10.7 Hz, J₂=2.1 Hz), 124.2, 123.8, 114.32 and 114.29 and 114.26 and 114.23 (dd, J₁=9.5 Hz, J₂=5.0 Hz), 110.87 and 110.85 and 110.72 and 110.71 (dd, J₁=21.8 Hz, J₂=2.6 Hz), 103.8 and 103.7 and 103.6 and 103.5 (dd, J₁=27.0 Hz, J₂=22.6 Hz); HRMS m/z calcd for C₁₂H₈BrF₂N₃O [M+H]⁺: 327.9897; found 327.9890. The purity of the compound was further confirmed by HPLC: Rₜ=16.28 min (99% pure).

### Example 22: Synthesis of Compound 5c (SGT1798)

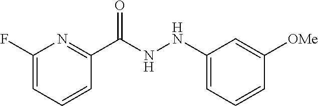 [SMILES: COc1cccc(NNC(=O)c2cccc(F)n2)c1]

To a solution of 6-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 3-methoxyphenylhydrazine hydrochloride (161 mg, 0.92 mmol). The reaction mixture was stirred at room temperature for 12 h, and the progress of the reaction was monitored by TLC (2:3/EtOAc:Hexanes, R_{f}0.42). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 5c (85 mg, 46%) as a yellow solid: ¹H NMR (500 MHZ, (CD₃)₂SO) δ 10.54 (d, J=2.8 Hz, 1H), 8.20 (td, J₁=8.2 Hz, J₂=7.4 Hz, 1H), 7.95 (ddd, J₁=7.5 Hz, J₂=2.4 Hz, J₃=0.8 Hz, 1H), 7.92 (d, J=2.7 Hz, 1H), 7.46 (ddd, J₁=8.3 Hz, J₂=2.4 Hz, J₃=0.8 Hz, 1H), 7.04 (t, J=8.1 Hz, 1H), 6.35 (ddd, J₁=8.1 Hz, J₂=2.0 Hz, J₃=1.0 Hz, 1H), 6.32-6.29 (m, 2H), 3.67 (s, 3H); ¹³C NMR (150 MHZ, (CD₃)₂SO) δ 162.9, 162.6 and 161.0 (d, J=238.3 Hz), 160.1, 150.5, 148.23 and 148.15 (d, J=11.9 Hz), 143.74 and 143.69 (d, J=7.7 Hz), 129.6, 120.5, 113.3 and 113.1 (d, J=36.2 Hz), 105.2 and 104.0 (d, J=178.7 Hz), 98.4, 54.8; HRMS m/z calcd for C₁₃H₁₂FN₃O₂ [M+H]⁺: 262.0992; found 262.0986. The purity of the compound was further confirmed by HPLC: Rₜ=15.29 min (96% pure).

### Example 23: Synthesis of Compound 5d (SGT1800)

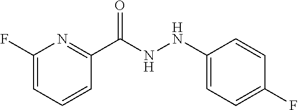 [SMILES: O=C(NNc1ccc(F)cc1)c1cccc(F)n1]

To a solution of 6-fluoro-2-pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 4-fluorophenylhydrazine hydrochloride (150 mg, 0.92 mmol). The reaction mixture was stirred at room temperature for 12 h, and the progress of the reaction was monitored by TLC (2:3/EtOAc:Hexanes, R/0.49). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 5d (122 mg, 69%) as a yellow solid: ¹H NMR (500 MHZ, (CD₃)₂SO) δ 10.60 (d, J=3.0 Hz, 1H), 8.20 (td, J₁=8.2 Hz, J₂=7.4 Hz, 1H), 7.95 (ddd, J₁=7.5 Hz, J₂=2.4 Hz, J₂=0.8 Hz, 1H), 7.89 (d, J=3.2 Hz, 1H), 7.47 (ddd, J₁=8.3 Hz, J₂=2.5 Hz, J₂=0.8 Hz, 1H), 6.99 (t, J=8.9 Hz, 2H), 6.76 (dd, J₁=9.1 Hz, J₂=4.7 Hz, 2H); ¹³C NMR (100 MHZ, (CD₃)₂SO) δ 162.9 and 160.5 (d, J=238.3 Hz), 162.8, 157.1 and 154.7 (d, J=232.3 Hz), 148.2 and 148.0 (d, J=12.1 Hz), 145.60 and 145.58 (d, J=1.9 Hz), 143.7 and 143.6 (d, J=7.9 Hz), 120.50 and 120.46 (d, J=3.7 Hz), 115.2 and 115.0 (d, J=22.3 Hz), 113.64 and 113.56 (d, J-7.6 Hz), 113.3 and 113.0 (d, J=36.3 Hz); HRMS m/z calcd for C₁₂H₉F₂N₃O [M+H]⁺: 250.0792; found 250.0785. The purity of the compound was further confirmed by HPLC: Rₜ=15.36 min (96% pure).

### Example 24: Synthesis of Compound 5f (SGT1770)

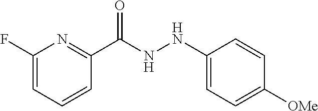 [SMILES: COc1ccc(NNC(=O)c2cccc(F)n2)cc1]

To a solution of 6-fluoro-2-pyridinecarboxylic acid (100 mg, 0.71 mmol) in DMF (3 mL) at 0° C., N-(3-dimethylaminopropyl)-N′-ethylcarbodiimide hydrochloride (163 mg, 0.85 mmol), 1-hydroxybenzotriazole hydrate (115 mg, 0.85 mmol), and N,N-diisopropylethyl amine (0.37 mL, 2.13 mmol) were added. The reaction mixture was stirred at 0° C. for 30 min followed by the addition of 4-methoxyphenylhydrazine hydrochloride (148 mg, 0.85 mmol). The reaction mixture was stirred at room temperature for 12 h, and the progress of the reaction was monitored by TLC (2:3/EtOAc:Hexanes, R/0.49). The reaction was quenched with H₂O (100 mL) and extracted with EtOAc (70 mL). The organic layer was washed with H₂O (70 mL), brine (20 mL), dried over Na₂SO₄, and concentrated under reduced pressure. The resulting residue was purified by flash column chromatography (SiO₂, 2:3/EtOAc:Hexanes) to afford compound 5f (109 mg, 59%) as a pale yellow solid: ¹H NMR (500 MHZ, CD₃OD) δ 8.13 (td, J₁=8.1 Hz, J₂=7.5 Hz, 1H), 8.02 (ddd, J₁=7.4 Hz, J₂=2.1 Hz, J₃=0.8 Hz, 1H), 7.31 (ddd, J₁=8.2 Hz, J₂=2.5 Hz, J₃=0.8 Hz, 1H), 6.85 (d, J=9.2 Hz, 2H), 6.80 (d, J=9.3 Hz, 2H), 3.72 (s, 3H); ¹³C NMR (150 MHz, (CD₃)₂SO) δ 162.7, 162.5 and 160.9 (d, J=238.1 Hz), 152.8, 148.3 and 148.2 (d, J=11.9 Hz), 143.7 and 143.6 (d, J=7.6 Hz), 142.9, 120.43 and 120.41 (d, J=3.9 Hz), 114.2, 114.0, 113.2 and 112.9 (d, J=36.0 Hz), 55.3; HRMS m/z calcd for C₁₃H₁₂FN₃O₂ [M+H]⁺: 262.0992; found 262.0983. The purity of the compound was further confirmed by HPLC: Rₜ=15.01 min (97% pure).

### Example 25: Synthesis of Compound 5g (SGT1804)

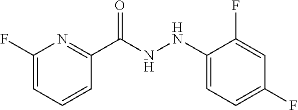 [SMILES: O=C(NNc1ccc(F)cc1F)c1cccc(F)n1]

To a solution of 6-fluoro-2-F pyridinecarboxylic acid (100 mg, 0.71 mmol) in DMF (3 mL) at 0° C., N-(3-dimethylaminopropyl)-N′-ethylcarbodiimide hydrochloride (176 mg, 0.92 mmol), 1-hydroxybenzotriazole hydrate (124 mg, 0.92 mmol), and N,N-diisopropylethyl amine (0.37 mL, 2.13 mmol) were added. The reaction mixture was stirred at 0° C. for 30 min followed by the addition of 2,4-difluorophenylhydrazine hydrochloride (166 mg, 0.92 mmol). The reaction mixture was stirred at room temperature for 12 h, and the progress of the reaction was monitored by TLC (2:3/EtOAc:Hexanes, R/0.46). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 5g (133 mg, 70%) as a pale yellow solid: ¹H NMR (500 MHZ, (CD₃)₂SO) δ 10.63 (s, 1H), 8.21 (td, J₁=8.1 Hz, J₂=7.2 Hz, 1H), 7.96 (dd, J₁=7.5 Hz, J₂=1.6 Hz, 1H), 7.79 (s, 1H), 7.48 (dd, J₁=8.3 Hz, J₂=2.5 Hz, 1H), 7.16 (ddd, J₁=11.8 Hz, J₂=9.0 Hz, J₃=2.8 Hz, 1H), 6.91-6.85 (m, 1H), 6.77 (td, J₁=9.8 Hz, J₂=5.8 Hz, 1H); ¹³C NMR (150 MHz, (CD₃)₂SO) δ 163.0, 162.6 and 161.0 (d, J=238.3 Hz), 155.7 and 155.6 and 154.1 and 154.0 (dd, J₁=235.1 Hz, J₂=10.7 Hz), 150.5 and 150.4 and 148.9 and 148.8 (dd, J₁=240.5 Hz, J₂=11.9 Hz), 147.99 and 147.91 (d, J=12.1 Hz), 143.81 and 143.75 (d, J=7.7 Hz), 133.50 and 133.48 and 133.43 and 133.41 (dd, J₁=10.8 Hz, J₂=3.2 Hz), 120.61 and 120.59 (d, J=3.7 Hz), 114.40 and 114.37 and 114.34 and 114.31 (dd, J₁=8.7 Hz, J₂=4.3 Hz), 113.5 and 113.2 (d, J=36.0 Hz), 110.93 and 110.91 and 110.78 and 110.76 (dd, J₁=21.7 Hz, J₂=3.3 Hz), 103.9 and 103.71 and 103.68 and 103.5 (dd, J₁=26.9 Hz, J₂=22.5 Hz); HRMS m/z calcd for C₁₂H₈F₃N₃O [M+H]⁺: 268.0697; found 268.0692. The purity of the compound was further confirmed by HPLC: Rₜ=15.68 min (99% pure).

### Example 26: Synthesis of Compound 6c (SGT1437)

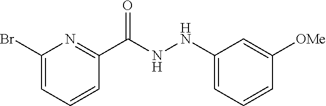 [SMILES: COc1cccc(NNC(=O)c2cccc(Br)n2)c1]

To a solution of 6-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 3-methoxyphenylhydrazine hydrochloride (112 mg, 0.64 mmol). The reaction mixture was stirred at room temperature for 12 h, and the progress of the reaction was monitored by TLC (2:3/EtOAc:Hexanes, R/0.41). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 6c (98 mg, 61%) as a pale yellow solid: ¹H NMR (500 MHZ, (CD₃)₂SO) δ 10.49 (d, J=2.7 Hz, 1H), 8.01 (dd, J₁=7.6 Hz, J₂=1.1 Hz, 1H), 7.96 (t, J=7.8 Hz, 1H), 7.92 (d, J=2.5 Hz, 1H), 7.90 (dd, J₁=7.8 Hz, J₂=1.1 Hz, 1H), 7.05 (dd, J₁=8.6 Hz, J₂=8.5 Hz, 1H), 6.35 (ddd, J₁=8.1 Hz, J₂=2.1 Hz, J₃=1.0 Hz, 1H), 6.33-6.29 (m, 2H), 3.67 (s, 3H); ¹³C NMR (150 MHZ, (CD₃)₂SO) δ 162.8, 160.1, 151.0, 150.5, 140.9, 140.4, 131.2, 129.5, 121.9, 105.2, 104.0, 98.4, 54.8; HRMS m/z calcd for C₁₃H₁₂BrN₃O₂ [M+H]⁺: 322.0191; found 322.0187. The purity of the compound was further confirmed by HPLC: Rₜ=16.05 min (96% pure).

### Example 27: Synthesis of Compound 6d (SGT1775)

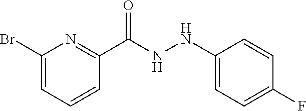 [SMILES: O=C(NNc1ccc(F)cc1)c1cccc(Br)n1]

To a solution of 6-bromo-2-F pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (88 mg, 0.64 mmol), and N,N-diisopropylethyl amine (0.26 mL, 1.50 mmol) were added. The reaction mixture was stirred at 0° C. for 30 min followed by the addition of 4-fluorophenylhydrazine hydrochloride (104 mg, 0.64 mmol). The reaction mixture was stirred at room temperature for 12 h, and the progress of the reaction was monitored by TLC (2:3/EtOAc:Hexanes, R/0.68). The reaction was quenched with H₂O (80 mL) and extracted with EtOAc (70 mL). The organic layer was washed with H₂O (60 mL), brine (20 mL), dried over MgSO₄, and concentrated under reduced pressure. The resulting residue was purified by flash column chromatography (SiO₂, 3:7/EtOAc:Hexanes) to afford compound 6d (70 mg, 45%) as a white solid: ¹H NMR (400 MHZ, (CD₃)₂SO) δ 10.54 (d, J=3.2 Hz, 1H), 8.02 (dd, J₁=7.5 Hz, J₂=1.2 Hz, 1H), 7.96 (t, J=7.8 Hz, 1H), 7.89 (dd, J₁=7.8 Hz, J₂=1.2 Hz, 1H), 7.88 (s, 1H), 6.99 (t, J=8.9 Hz, 2H), 6.76 (dd, J₁=9.0 Hz, J₂=4.6 Hz, 2H); ¹³C NMR (100 MHZ, (CD₃)₂SO) δ 162.9, 156.8 and 155.2 (d, J=154.5 Hz), 151.0, 145.61 and 145.60 (d, J=1.0 Hz), 141.0, 140.4, 131.3, 122.0, 115.3 and 115.1 (d, J=15.0 Hz), 113.73 and 113.68 (d, J=5.0 Hz); HRMS m/z calcd for C₁₂H₉BrFN₃O [M+H]⁺: 309.9991; found 309.9966. The purity of the compound was further confirmed by HPLC: Rₜ=15.94 min (100% pure).

### Example 28: Synthesis of Compound 6f (SGT1435)

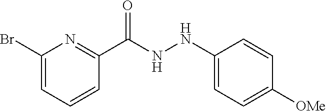 [SMILES: COc1ccc(NNC(=O)c2cccc(Br)n2)cc1]

To a solution of 6-bromo-2-pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 4-methoxyphenylhydrazine hydrochloride (112 mg, 0.64 mmol). The reaction mixture was stirred at room temperature for 12 h, and the progress of the reaction was monitored by TLC (2:3/EtOAc:Hexanes, R/0.40). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 6f (118 mg, 73%) as a pale yellow solid: ¹H NMR (500 MHZ, (CD₃)₂SO) δ 10.47 (d, J=3.6 Hz, 1H), 8.01 (dd, J₁=7.5 Hz, J₂=1.1 Hz, 1H), 7.95 (t, J=7.5 Hz, 1H), 7.89 (dd, J₁=7.9 Hz, J₂=1.1 Hz, 1H), 7.58 (d, J=3.5 Hz, 1H), 6.77 (d, J=9.4 Hz, 2H), 6.73 (d, J=9.3 Hz, 2H), 3.66 (s, 3H); ¹³C NMR (150 MHz, (CD₃)₂SO) δ 162.7, 152.8, 151.1, 142.8, 140.9, 140.4, 131.1, 121.8, 114.2, 114.0, 55.3; HRMS m/z calcd for C₁₃H₁₂BrN₃O₂ [M+H]⁺: 322.0191; found 322.0151. The purity of the compound was further confirmed by HPLC: Rₜ=15.87 min (98% pure).

### Example 29: Synthesis of Compound 6g (SGT1439)

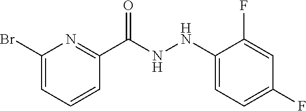 [SMILES: O=C(NNc1ccc(F)cc1F)c1cccc(Br)n1]

To a solution of 6-bromo-2-F pyridinecarboxylic acid (100 mg, 0.50 mmol) in DMF (3 mL) at 0° C., N-(3-dimethylaminopropyl)-N′-ethylcarbodiimide hydrochloride (123 mg, 0.64 mmol), 1-hydroxybenzotriazole hydrate (87 mg, 0.64 mmol), and N,N-diisopropylethyl amine (0.26 mL, 1.50 mmol) were added. The reaction mixture was stirred at 0° C. for 30 min followed by the addition of 2,4-difluorophenylhydrazine hydrochloride (116 mg, 0.64 mmol). The reaction mixture was stirred at room temperature for 12 h, and the progress of the reaction was monitored by TLC (2:3/EtOAc:Hexanes, R/0.44). The reaction was quenched with H₂O (60 mL) and extracted with EtOAc (80 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 6g (116 mg, 71%) as a white solid: ¹H NMR (500 MHZ, (CD₃)₂SO) δ 10.58 (s, 1H), 8.02 (dd, J₁=7.5 Hz, J₂=1.1 Hz, 1H), 7.96 (t, J=7.8 Hz, 1H), 7.91 (dd, J₁=7.9 Hz, J₂=1.2 Hz, 1H), 7.79 (s, 1H), 7.17 (ddd, J₁=11.8 Hz, J₂=8.9 Hz, J₃=2.8 Hz, 1H), 6.91-6.85 (m, 1H), 6.80 (td, J₁=9.6 Hz, J₂=5.8 Hz, 1H); ¹³C NMR (150 MHz, (CD₃)₂SO) δ 162.9, 155.63 and 155.56 and 154.1 and 154.0 (dd, J₁=235.1 Hz, J₂=10.8 Hz), 150.8, 150.44 and 150.36 and 148.83 and 148.75 (dd, J₁=241.4 Hz, J₂=11.9 Hz), 140.9, 140.4, 133.43 and 133.41 and 133.36 and 133.34 (dd, J₁=10.8 Hz, J₂=3.2 Hz), 131.3, 122.0, 114.44 and 114.41 and 114.38 and 114.35 (dd, J₁=8.8 Hz, J₂=4.4 Hz), 110.89 and 110.86 and 110.74 and 110.72 (dd, J₁=21.6 Hz, J₂=3.5 Hz), 103.8 and 103.67 and 103.63 and 103.5 (dd, J₁=26.6 Hz, J₂=21.8 Hz); HRMS m/z calcd for C₁₂H₈BrF₂N₃O [M+H]⁺: 327.9897; found 327.9889. The purity of the compound was further confirmed by HPLC: Rₜ=16.19 min (100% pure).

### Example 30: Synthesis of Compound 7c (SGT1774)

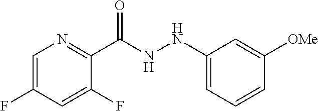 [SMILES: COc1cccc(NNC(=O)c2ncc(F)cc2F)c1]

To a solution of 3,5-difluoro-2-pyridinecarboxylic acid (125 mg, 0.79 mmol) in DMF (3 mL) at 0° C., N-(3-dimethylaminopropyl)-N′-ethylcarbodiimide hydrochloride (181 mg, 0.94 mmol), 1-hydroxybenzotriazole hydrate (127 mg, 0.94 mmol), and N,N-diisopropylethyl amine (0.41 mL, 2.37 mmol) were added. The reaction mixture was stirred at 0° C. for 30 min followed by the addition of 3-methoxyphenylhydrazine hydrochloride (164 mg, 0.94 mmol). The reaction mixture was stirred at room temperature for 12 h, and the progress of the reaction was monitored by TLC (2:3/EtOAc:Hexanes, R/0.42). The reaction was quenched with H₂O (100 mL) and extracted with EtOAc (70 mL). The organic layer was washed with H₂O (70 mL), brine (20 mL), dried over Na₂SO₄, and concentrated under reduced pressure. The resulting residue was purified by flash column chromatography (SiO₂, 2:3/EtOAc:Hexanes) to afford compound 7c (38 mg, 18%) as a pale yellow solid: ¹H NMR (500 MHz, CD₃OD) δ 8.49 (d, J=2.3 Hz, 1H), 7.80-7.74 (m, 1H), 7.09 (td, J₁=8.1 Hz, J₂=0.6 Hz, 1H), 6.49 (ddd, J₁=7.9 Hz, J₂=2.2 Hz, J₃=0.9 Hz, 1H), 6.48-6.47 (m, 1H), 6.40 (ddd, J₁=8.2 Hz, J₂=2.4 Hz, J₃=0.9 Hz, 1H), 3.74 (s, 3H); ¹³C NMR (150 MHz, (CD₃)₂SO) δ 162.2 and 162.1 (d, J=4.4 Hz), 160.74 and 160.70 and 158.99 and 158.95 (dd, J₁=261.2 Hz, J₂=5.6 Hz), 160.1, 158.2 and 158.1 and 156.4 and 156.3 (dd, J₁=267.5 Hz, J₂=7.5 Hz), 150.4, 136.86 and 136.84 and 136.79 and 136.76 (dd, J₁=10.7 Hz, J₂=4.2 Hz), 133.84 and 133.81 and 133.68 and 133.65 (dd, J₁=23.6 Hz, J₂=4.9 Hz), 129.6, 114.0 and 113.9 and 113.7 (t, J=4.4 Hz), 105.1, 104.0, 98.2, 54.8; HRMS m/z calcd for C₁₃H₁₁F₂N₃O₂ [M+H]⁺: 280.0897; found 280.0886. The purity of the compound was further confirmed by HPLC: Rₜ=14.89 min (95% pure).

### Example 31: Synthesis of Compound 7d (SGT1801)

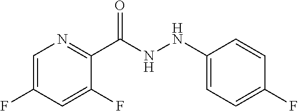 [SMILES: O=C(NNc1ccc(F)cc1)c1ncc(F)cc1F]

To a solution of 3,5-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-fluorophenylhydrazine hydrochloride (133 mg, 0.82 mmol). The reaction mixture was stirred at room temperature for 12 h, and the progress of the reaction was monitored by TLC (2:3/EtOAc:Hexanes, R/0.51). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 7d (109 mg, 65%) as a yellow solid: ¹H NMR (500 MHZ, (CD₃)₂SO) δ 10.47 (br s, 1H), 8.63 (m, 1H), 8.13 (ddd, J₁=10.5 Hz, J₂=9.2 Hz, J₂=2.4 Hz, 1H), 8.00 (br s, 1H), 7.02 (t, J=8.9 Hz, 2H), 6.79 (dd, J₁=9.1 Hz, J₂=4.7 Hz, 2H); ¹³C NMR (100 MHZ, (CD₃)₂SO) δ 162.14 and 162.09 (d, J=5.3 Hz), 161.23 and 161.17 and 158.7 and 158.6 (dd, J₁=251.9 Hz, J₂=6.1 Hz), 158.61 and 158.55 and 156.03 and 155.96 (dd, J₁=258.5 Hz, J₂=6.2 Hz), 157.1 and 154.8 (d, J=232.3 Hz), 145.51 and 145.49 (d, J=1.8 Hz), 136.65 and 136.61 and 136.54 and 136.50 (dd, J₁=10.2 Hz, J₂=4.1 Hz), 133.82 and 133.78 and 133.6 and 133.5 (dd, J₁=23.3 Hz, J₂=4.5 Hz), 115.3 and 115.1 (d, J=22.2 Hz), 114.1 and 113.9 and 113.7 (t, J=22.1 Hz), 113.5 and 113.4 (d, J=7.6 Hz); HRMS m/z calcd for C₁₂H₈F₃N₃O [M+H]⁺: 268.0697; found 268.0690. The purity of the compound was further confirmed by HPLC: Rₜ=15.24 min (95% pure).

### Example 32: Synthesis of Compound 7f (SGT1795)

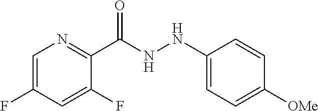 [SMILES: COc1ccc(NNC(=O)c2ncc(F)cc2F)cc1]

To a solution of 3,5-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-methoxyphenylhydrazine hydrochloride (143 mg, 0.82 mmol). The reaction mixture was stirred at room temperature for 12 h, and the progress of the reaction was monitored by TLC (2:3/EtOAc:Hexanes, R/0.57). The reaction was quenched with H₂O (80 mL) and extracted with EtOAc (70 mL). The organic layer was washed with H₂O (60 mL), brine (20 mL), dried over MgSO₄, and concentrated under reduced pressure. The resulting residue was purified by flash column chromatography (SiO₂, 3:7/EtOAc:Hexanes) to afford compound 7f (72 mg, 41%) as a white solid: ¹H NMR (500 MHZ, (CD₃)₂SO) δ 10.40 (d, J=3.4 Hz, 1H), 8.62 (d, J=2.3 Hz, 1H), 8.12 (ddd, J₁=10.6 Hz, J₂=9.2 Hz, J₃=2.4 Hz, 1H), 7.69 (d, J=3.8 Hz, 1H), 6.79 (d, J=9.3 Hz, 2H), 6.76 (d, J=9.5 Hz, 2H), 3.67 (s, 3H); ¹³C NMR (100 MHZ, (CD₃)₂SO) δ 162.10 and 162.05 (d, J=5.1 Hz), 161.15 and 161.09 (d, J=6.1 Hz), 158.6 and 158.54 and 158.47 (t, J=6.5 Hz), 155.93 and 155.86 (d, J=7.1 Hz), 152.8, 142.8, 136.90 and 136.86 and 136.80 and 136.76 (dd, J₁=10.5 Hz, J₂=4.1 Hz), 133.8 and 133.7 and 133.6 and 133.5 (dd, J₁=23.3 Hz, J₂=4.6 Hz), 114.2 and 113.8 (d, J=48.8 Hz), 114.1 and 113.9 and 113.6 (t, J=22.2 Hz), 55.3; HRMS m/z calcd for C₁₃H₁₁F₂N₃O₂ [M+H]⁺: 280.0897; found 280.0890. The purity of the compound was further confirmed by HPLC: Rₜ=14.98 min (97% pure).

### Example 33: Synthesis of Compound 7g (SGT1805)

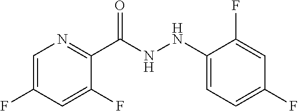 [SMILES: O=C(NNc1ccc(F)cc1F)c1ncc(F)cc1F]

To a solution of 3,5-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 2,4-difluorophenylhydrazine hydrochloride (148 mg, 0.82 mmol). The reaction mixture was stirred at room temperature for 12 h, and the progress of the reaction was monitored by TLC (2:3/EtOAc:Hexanes, R/0.53). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 7g (118 mg, 66%) as a pale yellow solid: ¹H NMR (500 MHZ, (CD₃)₂SO) δ 10.52 (s, 1H), 8.63 (d, J=2.4 Hz, 1H), 8.14 (ddd, J₁=10.5 Hz, J₂=9.1 Hz, J₃=2.4 Hz, 1H), 7.91 (s, 1H), 7.18 (ddd, J₁=11.8 Hz, J₂=9.0 Hz, J₃=2.8 Hz, 1H), 6.96-6.90 (m, 1H), 6.84 (td, J₁=9.5 Hz, J₂=5.8 Hz, 1H); ¹³C NMR (150 MHZ, (CD₃)₂SO) δ 162.13 and 162.09 (d, J=5.3 Hz), 160.91 and 160.87 and 159.2 and 159.1 (dd, J₁=261.2 Hz, J₂=5.7 Hz), 158.42 and 158.37 and 156.63 and 156.58 (dd, J₁=268.6 Hz, J₂=7.5 Hz), 155.7 and 155.6 and 154.1 and 154.0 (dd, J₁=235.3 Hz, J₂=10.3 Hz), 150.44 and 150.36 and 148.83 and 148.75 (dd, J₁=241.5 Hz, J₂=12.6 Hz), 136.28 and 136.25 and 136.21 and 136.19 (dd, J₁=9.8 Hz, J₂=4.3 Hz), 133.83 and 133.80 and 133.7 and 133.6 (dd, J₁=22.9 Hz, J₂=4.4 Hz), 133.40 and 133.38 and 133.32 and 133.31 (dd, J₁=11.4 Hz, J₂=2.6 Hz), 114.2 and 113.99 and 113.96 and 113.89 (dd, J₁=33.4 Hz, J₂=29.0 Hz), 114.0, 110.95 and 110.92 and 110.80 and 110.78 (dd, J₁=21.6 Hz, J₂=3.3 Hz), 103.9 and 103.79 and 103.76 and 103.6 (dd, J₁=26.9 Hz, J₂=22.0 Hz); HRMS m/z calcd for C₁₂H₇F₄N₃O [M+H]⁺: 286.0603; found 286.0591. The purity of the compound was further confirmed by HPLC: Rₜ=15.35 min (95% pure).

### Example 34: Synthesis of Compound 8c (SGT1775)

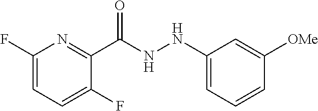 [SMILES: COc1cccc(NNC(=O)c2nc(F)ccc2F)c1]

To a solution of 3,6-difluoro-2-pyridinecarboxylic acid (125 mg, 0.79 mmol) in DMF (3 mL) at 0° C., N-(3-dimethylaminopropyl)-N′-ethylcarbodiimide hydrochloride (181 mg, 0.94 mmol), 1-hydroxybenzotriazole hydrate (127 mg, 0.94 mmol), and N,N-diisopropylethyl amine (0.41 mL, 2.37 mmol) were added. The reaction mixture was stirred at 0° C. for 30 min followed by the addition of 3-methoxyphenylhydrazine hydrochloride (164 mg, 0.94 mmol). The reaction mixture was stirred at room temperature for 12 h, and the progress of the reaction was monitored by TLC (2:3/EtOAc:Hexanes, R/0.41). The reaction was quenched with H₂O (100 mL) and extracted with EtOAc (70 mL). The organic layer was washed with H₂O (70 mL), brine (20 mL), dried over Na₂SO₄, and concentrated under reduced pressure. The resulting residue was purified by flash column chromatography (SiO₂, 2:3/EtOAc:Hexanes) to afford compound 8c (43 mg, 19%) as a pale yellow solid: ¹H NMR (500 MHZ, CD₃OD) δ 7.49 (td, J₁=9.0 Hz, J₂=5.6 Hz, 1H), 7.35 (ddd, J₁=9.0 Hz, J₂=3.6 Hz, J₃=3.1 Hz, 1H), 7.09 (td, J₁=7.8 Hz, J₂=0.7 Hz, 1H), 6.49 (ddd, J₁=8.8 Hz, J₂=2.2 Hz, J₃=0.9 Hz, 1H), 6.48-6.47 (m, 1H), 6.41 (ddd, J₁=8.2 Hz, J₂=2.5 Hz, J₃=0.9 Hz, 1H), 3.75 (s, 3H); ¹³C NMR (150 MHz, (CD₃)₂SO) δ 161.6 and 161.5 (d, J=4.4 Hz), 160.2, 158.0 and 156.4 (d, J=235.9 Hz), 155.94 and 155.91 and 154.23 and 154.21 (dd, J₁=260.0 Hz, J₂=3.7 Hz), 150.3, 136.7 and 136.6 and 136.5 (t, J=15.0 Hz), 132.05 and 131.99 and 131.90 and 131.8 (dd, J₁=22.8 Hz, J₂=8.7 Hz), 129.6, 114.4 and 114.3 and 114.11 and 114.07 (dd, J₁=41.0 Hz, J₂=6.1 Hz), 105.1, 104.2, 98.2, 54.8; HRMS m/z calcd for C₁₃H₁₁F₂N₃O₂ [M+H]⁺: 280.0897; found 280.0886. The purity of the compound was further confirmed by HPLC: Rₜ=14.91 min (99% pure).

### Example 35: Synthesis of Compound 8d (SGT1802)

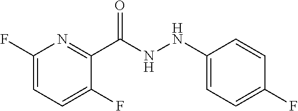 [SMILES: O=C(NNc1ccc(F)cc1)c1nc(F)ccc1F]

To a solution of 3,6-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-fluorophenylhydrazine hydrochloride (133 mg, 0.82 mmol). The reaction mixture was stirred at room temperature for 12 h, and the progress of the reaction was monitored by TLC (2:3/EtOAc:Hexanes, R_{f}0.50). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 8d (116 mg, 69%) as a yellow solid: ¹H NMR (500 MHZ, (CD₃)₂SO) δ 10.51 (s, 1H), 8.14 (td, J₁=8.9 Hz, J₂=6.0 Hz, 1H), 8.01 (s, 1H), 7.53 (dt, J₁=8.6 Hz, J₂=3.2 Hz, 1H), 7.02 (t, J=8.9 Hz, 2H), 6.78 (dd, J₁=9.1 Hz, J₂=4.6 Hz, 2H); ¹³C NMR (100 MHZ, (CD₃)₂SO) δ 161.6 and 161.5 (d, J=4.7 Hz), 158.3 and 155.9 (d, J=120.9 Hz), 157.2 and 154.8 (d, J=232.6 Hz), 156.5 and 156.5 and 153.89 and 153.85 (dd, J₁=256.8 Hz, J₂=4.2 Hz), 145.35 and 145.33 (d, J=1.8 Hz), 136.5 and 136.4 and 136.3 and 136.2 (dd, J₁=15.6 Hz, J₂=13.9 Hz), 132.2 and 132.1 and 131.9 and 131.8 (dd, J₁=22.9 Hz, J₂=8.8 Hz), 115.3 and 115.1 (d, J=89.2 Hz), 114.6 and 114.5 and 114.2 and 114.1 (dd, J₁=41.0 Hz, J₂=6.1 Hz), 113.5 and 113.4 (d, J=30.4 Hz); HRMS m/z calcd for C₁₂H₉₈F₃N₃O [M+H]⁺: 268.0697; found 268.0695. The purity of the compound was further confirmed by HPLC: Rₜ=15.12 min (95% pure).

### Example 36: Synthesis of Compound 8f (SGT1796)

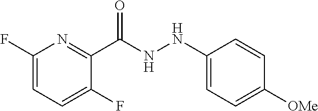 [SMILES: COc1ccc(NNC(=O)c2nc(F)ccc2F)cc1]

To a solution of 3,6-difluoro-2-pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 4-methoxyphenylhydrazine hydrochloride (143 mg, 0.82 mmol). The reaction mixture was stirred at room temperature for 12 h, and the progress of the reaction was monitored by TLC (2:3/EtOAc:Hexanes, R/0.51). The reaction was quenched with H₂O (80 mL) and extracted with EtOAc (70 mL). The organic layer was washed with H₂O (60 mL), brine (20 mL), dried over MgSO₄, and concentrated under reduced pressure. The resulting residue was purified by flash column chromatography (SiO₂, 2:3/EtOAc:Hexanes) to afford compound 8f (74 mg, 42%) as an orange solid: ¹H NMR (500 MHZ, (CD₃)₂SO) δ 10.44 (d, J=3.6 Hz, 1H), 8.13 (td, J₁=8.9 Hz, J₂=6.0 Hz, 1H), 7.71 (d, J=3.6 Hz, 1H), 7.51 (ddd, J₁=9.0 Hz, J₂=3.4 Hz, J₃=2.8 Hz, 1H), 6.80 (d, J=9.2 Hz, 2H), 6.76 (d, J=9.1 Hz, 2H), 3.67 (s, 3H); ¹³C NMR (100 MHZ, (CD₃)₂SO) δ 161.50 and 161.46 (d, J=4.7 Hz), 158.32 and 158.31 and 155.96 and 155.95 (dd, J₁=235.9 Hz, J₂=1.0 Hz), 156.35 and 156.31 and 153.78 and 153.74 (dd, J₁=256.2 Hz, J₂=4.1 Hz), 153.4, 152.9, 142.6, 141.9, 136.8 and 136.7 and 136.6 and 136.5 (dd, J₁=15.9 Hz, J₂=13.9 Hz), 132.1 and 132.0 and 131.84 and 131.75 (dd, J₁=23.0 Hz, J₂=8.9 Hz), 114.4 and 114.2 and 114.0 and 113.9 (dd, J₁=17.3 Hz, J₂=6.0 Hz), 114.3 and 113.8 (d, J=49.3 Hz); HRMS m/z calcd for C₁₃H₁₁F₂N₃O₂ [M+H]⁺: 280.0897; found 280.0885. The purity of the compound was further confirmed by HPLC: Rₜ=15.02 min (97% pure).

### Example 37: Synthesis of Compound 8g (SGT1806)

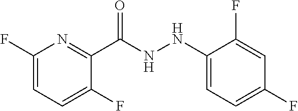 [SMILES: O=C(NNc1ccc(F)cc1F)c1nc(F)ccc1F]

To a solution of 3,6-difluoro-2-F pyridinecarboxylic acid (100 mg, 0.63 mmol) in DMF (3 mL) at 0° C., N-(3-dimethylaminopropyl)-N′-ethylcarbodiimide hydrochloride (157 mg, 0.82 mmol), 1-hydroxybenzotriazole hydrate (111 mg, 0.82 mmol), and N,N-diisopropylethyl amine (0.33 mL, 1.89 mmol) were added. The reaction mixture was stirred at 0° C. for 30 min followed by the addition of 2,4-difluorophenylhydrazine hydrochloride (148 mg, 0.82 mmol). The reaction mixture was stirred at room temperature for 12 h, and the progress of the reaction was monitored by TLC (2:3/EtOAc:Hexanes, R/0.51). The reaction was quenched with H₂O (80 mL) and extracted with EtOAc (60 mL). The organic layer was washed with H₂O (50 mL), brine (20 mL), dried over MgSO₄, and concentrated under reduced pressure. The resulting residue was purified by flash column chromatography (SiO₂, 2:3/EtOAc:Hexanes) to afford compound 8g (109 mg, 61%) as a pale yellow solid: ¹H NMR (500 MHZ, (CD₃)₂SO) δ 10.55 (s, 1H), 8.15 (td, J₁=9.0 Hz, J₂=6.0 Hz, 1H), 7.93 (s, 1H), 7.53 (dt, J₁=8.9 Hz, J₂=2.9 Hz, 1H), 7.18 (ddd, J₁=11.8 Hz, J₂=8.9 Hz, J₃=2.8 Hz, 1H), 6.97-6.91 (m, 1H), 6.84 (td, J₁=9.5 Hz, J₂=5.8 Hz, 1H); ¹³C NMR (150 MHz, (CD₃)₂SO) δ 161.6 and 161.5 (d, J=4.7 Hz), 157.9 and 156.3 (d, J=236.0 Hz), 156.2 and 156.1 and 154.44 and 154.41 (dd, J₁=256.9 Hz, J₂=4.3 Hz), 155.72 and 155.65 and 154.15 and 154.08 (dd, J₁=235.1 Hz, J₂=10.7 Hz), 150.5 and 150.4 and 148.84 and 148.76 (dd, J₁=241.5 Hz, J₂=12.0 Hz), 136.1 and 136.0 and 135.9 (t, J=14.3 Hz), 133.23 and 133.21 and 133.15 and 133.14 (dd, J₁=10.8 Hz, J₂=2.3 Hz), 132.24 and 132.19 and 132.1 and 132.03 (dd, J₁=22.9 Hz, J₂=8.8 Hz), 114.72 and 114.68 and 114.45 and 114.41 (dd, J₁=41.0 Hz, J₂=6.3 Hz), 114.1 and 114.03 and 114.00 and 113.97 (dd, J₁=9.0 Hz, J₂=4.4 Hz), 110.98 and 110.95 and 110.83 and 110.81 (dd, J₁=21.7 Hz, J₂=3.7 Hz), 104.0 and 103.81 and 103.78 and 103.6 (dd, J₁=26.5 Hz, J₂=22.1 Hz); HRMS m/z calcd for C₁₂H₇F₄N₃O [M+H]⁺: 286.0603; found 286.0600. The purity of the compound was further confirmed by HPLC: Rₜ=15.38 min (98% pure).

### Example 38: Synthesis of Compound 9b (SGT1793)

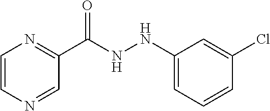 [SMILES: O=C(NNc1cccc(Cl)c1)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol) and N,N-diisopropylethyl amine (0.40 mL, 2.43 mmol) were added. The reaction mixture was stirred at 0° C. for 30 min followed by the addition of 3-chlorophenylhydrazine hydrochloride (188 mg, 1.05 mmol). The reaction mixture was stirred at room temperature for 12 h, and the progress of the reaction was monitored by TLC (3:2/EtOAc:Hexanes, R/0.31). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9b (137 mg, 68%) as a pale yellow solid: ¹H NMR (500 MHZ, (CD₃)₂SO) δ 10.83 (s, 1H), 9.18 (d, J=1.5 Hz, 1H), 8.91 (d, J=2.5 Hz, 1H), 8.78 (dd, J₁=2.3 Hz, J₂=1.5 Hz, 1H), 8.29 (s, 1H), 7.18-7.13 (m, 1H), 6.76-6.70 (m, 3H); ¹³C NMR (150 MHz, (CD₃)₂SO) δ 163.1, 150.6, 147.9, 144.6, 143.7, 143.6, 133.4, 130.4, 118.1, 111.5, 111.0; HRMS m/z calcd for C₁₁H₉ClN₄O [M+H]⁺: 249.0543; found 249.0546. The purity of the compound was further confirmed by HPLC: Rₜ=15.00 min (97% pure).

### Example 39: Synthesis of Compound 9c (SGT1773)

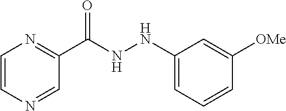 [SMILES: COc1cccc(NNC(=O)c2cnccn2)c1]

To a solution of pyrazinecarboxylic acid (125 mg, 1.01 mmol) in DMF (3 mL) at 0° C., N-(3-dimethylaminopropyl)-N′-ethylcarbodiimide hydrochloride (232 mg, 1.21 mmol), 1-hydroxybenzotriazole hydrate (163 mg, 1.21 mmol), and N,N-diisopropylethyl amine (0.53 mL, 3.03 mmol) were added. The reaction mixture was stirred at 0° C. for 30 min followed by the addition of 3-methoxyphenylhydrazine hydrochloride (211 mg, 1.21 mmol). The reaction mixture was stirred at room temperature for 12 h, and the progress of the reaction was monitored by TLC (2:3/EtOAc:Hexanes, R/0.21). The reaction was quenched with H₂O (100 mL) and extracted with EtOAc (70 mL). The organic layer was washed with H₂O (70 mL), brine (20 mL), dried over Na₂SO₄, and concentrated under reduced pressure. The resulting residue was purified by flash column chromatography (SiO₂, 3:2/EtOAc:Hexanes) to afford compound 9c (49 mg, 20%) as a pale yellow solid: ¹H NMR (500 MHz, CD₃OD) δ 9.24 (d, J=1.6 Hz, 1H), 8.83 (d, J=2.5 Hz, 1H), 8.73 (dd, J₁=2.5 Hz, J₂=1.5 Hz, 1H), 7.09 (t, J=8.1 Hz, 1H), 6.47 (ddd, J₁=8.0 Hz, J₂=2.2 Hz, J₃=0.9 Hz, 1H), 6.45 (t, J=2.3 Hz, 1H), 6.41 (ddd, J₁=8.2 Hz, J₂=2.5 Hz, J₃=0.9 Hz, 1H), 3.73 (s, 3H); ¹³C NMR (150 MHz, (CD₃)₂SO) δ 163.0, 160.1, 150.5, 147.8, 144.8, 143.62, 143.59, 129.6, 105.2, 104.0, 98.3, 54.8; HRMS m/z calcd for C₁₂H₁₂N₄O₂ [M+H]⁺: 245.1038; found 245.1027. The purity of the compound was further confirmed by HPLC: Rₜ=14.14 min (97% pure).

### Example 40: Synthesis of Compound 9d (SGT1780)

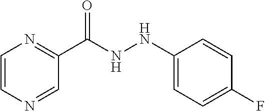 [SMILES: O=C(NNc1ccc(F)cc1)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.40 mL, 2.43 mmol) were added. The reaction mixture was stirred at 0° C. for 30 min followed by the addition of 4-fluorophenylhydrazine hydrochloride (171 mg, 1.05 mmol). The reaction mixture was stirred at room temperature for 12 h, and the progress of the reaction was monitored by TLC (1:2/EtOAc:Hexanes, R/0.21). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9d (154 mg, 82%) as a yellow solid: ¹H NMR (500 MHZ, (CD₃)₂SO) δ 10.78 (s, 1H), 9.16 (d, J=1.5 Hz, 1H), 8.90 (d, J=2.5 Hz, 1H), 8.77 (dd, J₁=2.5 Hz, J₂=1.5 Hz, 1H), 7.96 (s, 1H), 6.99 (t, J=9.0 Hz, 2H), 6.78 (dd, J₁=9.1 Hz, J₂=4.6 Hz, 2H); ¹³C NMR (150 MHz, (CD₃)₂SO) δ 163.1, 156.7 and 155.2 (d, J=231.8 Hz), 147.8, 145.5, 144.8, 143.63, 143.58, 115.2 and 115.1 (d, J=22.6 Hz), 113.7 and 113.6 (d, J=7.6 Hz); HRMS m/z calcd for C₁₁H₉FN₄O [M+H]⁺: 233.0838; found 233.0817. The purity of the compound was further confirmed by HPLC: Rₜ=14.38 min (97% pure).

### Example 41: Synthesis of Compound 9f (SGT1782)

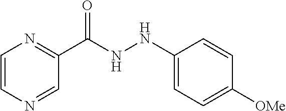 [SMILES: COc1ccc(NNC(=O)c2cnccn2)cc1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.40 mL, 2.43 mmol) were added. The reaction mixture was stirred at 0° C. for 30 min followed by the addition of 4-methoxyphenylhydrazine hydrochloride (183 mg, 1.05 mmol). The reaction mixture was stirred at room temperature for 12 h, and the progress of the reaction was monitored by TLC (1:2/EtOAc:Hexanes, R/0.263). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9f (122 mg, 62%) as a brown solid: ¹H NMR (500 MHZ, (CD₃)₂SO) δ 10.71 (d, J=3.7 Hz, 1H), 9.15 (d, J=1.6 Hz, 1H), 8.90 (d, J=2.5 Hz, 1H), 8.77 (dd, J₁=2.5 Hz, J₂=1.5 Hz, 1H), 7.65 (d, J=3.7 Hz, 1H), 6.76 (d, J=1.9 Hz, 4H), 3.66 (s, 3H); ¹³C NMR (150 MHz, (CD₃)₂SO) δ 163.0, 152.9, 147.7, 144.9, 143.6 (2C), 142.8, 114.2, 114.0, 55.3; HRMS m/z calcd for C₁₂H₁₂N₄O₂ [M+H]⁺: 245.1038; found 245.1011. The purity of the compound was further confirmed by HPLC: Rₜ=14.29 min (97% pure).

### Example 42: Synthesis of Compound 9g (SGT1397)

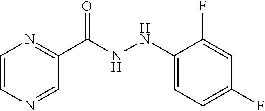 [SMILES: O=C(NNc1ccc(F)cc1F)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol), 1-hydroxybenzotriazole hydrate (142 mg, 1.05 mmol), and N,N-diisopropylethyl amine (0.40 mL, 2.43 mmol) were added. The reaction mixture was stirred at 0° C. for 30 min followed by the addition of 2,4-difluorophenylhydrazine hydrochloride (190 mg, 1.05 mmol). The reaction mixture was stirred at room temperature for 12 h, and the progress of the reaction was monitored by TLC (2:4/EtOAc:Hexanes, R/0.24). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 1:1/EtOAc:Hexanes) to afford compound 9g (160 mg, 79%) as a yellow solid: ¹H NMR (500 MHZ, (CD₃)₂SO) δ 10.82 (s, 1H), 9.16 (d, J=1.5 Hz, 1H), 8.91 (d, J=2.5 Hz, 1H), 8.78 (dd, J₁=2.5 Hz, J₂=1.5 Hz, 1H), 7.87 (s, 1H), 7.17 (ddd, J₁=11.8 Hz, J₂=9.0 Hz, J₃=2.8 Hz, 1H), 6.90-6.85 (m, 1H), 6.81 (td, J₁=9.5 Hz, J₂=5.8 Hz, 1H); ¹³C NMR (100 MHZ, (CD₃)₂SO) δ 163.2, 156.1 and 156.0 and 153.7 and 153.6 (dd, J₁=235.5 Hz, J₂=10.7 Hz), 150.9 and 150.8 and 148.5 and 148.4 (dd, J₁=241.4 Hz, J₂=12.0 Hz), 147.9, 144.6, 143.7, 143.6, 133.42 and 133.39 and 133.31 and 133.28 (dd, J₁=10.9 Hz, J₂=3.0 Hz), 114.5 and 114.41 and 114.36 and 114.32 (dd, J₁=9.1 Hz, J₂=4.6 Hz), 110.94 and 110.90 and 110.72 and 110.69 (dd, J₁=21.8 Hz, J₂=3.6 Hz), 103.9 and 103.72 and 103.67 and 103.4 (dd, J₁=26.8 Hz, J₂=22.3 Hz); HRMS m/z calcd for C₁₁H₈F₂N₄O [M+H]⁺: 251.0744; found 251.0723. The purity of the compound was further confirmed by HPLC: Rₜ=14.93 min (96% pure).

### Example 43: Synthesis of Compound 9h (SGT1791)

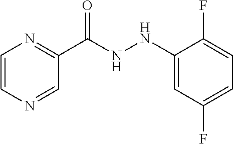 [SMILES: O=C(NNc1cc(F)ccc1F)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol) and N,N-diisopropylethyl amine (0.40 mL, 2.43 mmol) were added. The reaction mixture was stirred at 0° C. for 30 min followed by the addition of 2,5-difluorophenylhydrazine hydrochloride (187 mg, 1.05 mmol). The reaction mixture was stirred at room temperature for 12 h, and the progress of the reaction was monitored by TLC (3:2/EtOAc:Hexanes, R/0.28). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9h (156 mg, 77%) as a pale yellow solid: ¹H NMR (500 MHZ, (CD₃)₂SO) δ 10.83 (s, 1H), 9.18 (d, J=1.5 Hz, 1H), 8.92 (d, J=2.5 Hz, 1H), 8.78 (dd, J₁=2.5 Hz, J₂=1.5 Hz, 1H), 8.25 (s, 1H), 7.13 (ddd, J₁=13.9 Hz, J₂=8.9 Hz, J₃=5.0 Hz, 1H), 6.56 (ddd, J₁=10.4 Hz, J₂=5.4 Hz, J₃=3.2 Hz, 1H), 6.52-6.46 (m, 1H); ¹³C NMR (150 MHZ, (CD₃)₂SO) δ 163.2, 159.7 and 158.2 (d, J=235.8 Hz), 147.9, 147.0, 145.5, 144.6, 143.8 and 143.6 (d, J=27.0 Hz), 138.3 and 138.2 and 138.1 (t, J=11.7 Hz), 115.9 and 115.8 and 115.74 and 115.67 (dd, J₁=20.4 Hz, J₂=10.6 Hz), 103.93 and 103.88 and 103.77 and 103.72 (dd, J₁=24.6 Hz, J₂=7.5 Hz), 100.39 and 100.37 and 100.21 and 100.18 (dd, J₁=29.1 Hz, J₂=4.1 Hz); HRMS m/z calcd for C₁₁H₈F₂N₄O [M+H]⁺: 251.0744; found 251.0739. The purity of the compound was further confirmed by HPLC: Rₜ=14.89 min (98% pure).

### Example 44: Synthesis of Compound 9i (SGT1792)

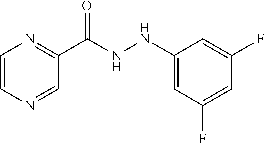 [SMILES: O=C(NNc1cc(F)cc(F)c1)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol) and N,N-diisopropylethyl amine (0.40 mL, 2.43 mmol) were added. The reaction mixture was stirred at 0° C. for 30 min followed by the addition of 3,5-difluorophenylhydrazine hydrochloride (187 mg, 1.05 mmol). The reaction mixture was stirred at room temperature for 12 h, and the progress of the reaction was monitored by TLC (3:2/EtOAc:Hexanes, R/0.26). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9i (148 mg, 73%) as a pale yellow solid: ¹H NMR (500 MHZ, (CD₃)₂SO) δ 10.87 (s, 1H), 9.18 (d, J=1.5 Hz, 1H), 8.91 (d, J=2.5 Hz, 1H), 8.78 (dd, J₁=2.5 Hz, J₂=1.5 Hz, 1H), 8.57 (s, 1H), 6.45 (tt, J₁=9.4 Hz, J₂=2.4 Hz, 1H), 6.40-6.35 (m, 2H); ¹³C NMR (150 MHZ, (CD₃)₂SO) δ 164.1 and 164.0 and 162.5 and 162.4 (dd, J₁=240.6 Hz, J₂=16.0 Hz), 163.1, 152.2 and 152.1 and 152.0 (t, J=12.9 Hz), 147.9, 144.5, 143.8 and 143.6 (d, J=31.6 Hz), 95.04 and 94.85 (d, J=28.7 Hz), 95.00 and 94.89 (d, J=16.5 Hz), 93.3 and 93.1 and 92.9 (t, J=26.3 Hz); HRMS m/z calcd for C₁₁H₈F₂N₄O [M+H]⁺: 251.0744; found 251.0738. The purity of the compound was further confirmed by HPLC: Rₜ=14.82 min (98% pure).

### Example 45: Synthesis of Compound 9j (SGT1794)

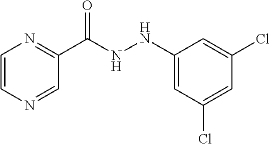 [SMILES: O=C(NNc1cc(Cl)cc(Cl)c1)c1cnccn1]

To a solution of pyrazinecarboxylic acid (100 mg, 0.81 mmol) in DMF (3 mL) at 0° C., N-(3-dimethylaminopropyl)-N′-ethylcarbodiimide hydrochloride (201 mg, 1.05 mmol) and N,N-diisopropylethyl amine (0.40 mL, 2.43 mmol) were added. The reaction mixture was stirred at 0° C. for 30 min followed by the addition of 3,5-dichlorophenylhydrazine hydrochloride (224 mg, 1.05 mmol). The reaction mixture was stirred at room temperature for 12 h, and the progress of the reaction was monitored by TLC (3:2/EtOAc:Hexanes, R_{f}0.36). The reaction was quenched with H₂O (100 mL), extracted with EtOAc (150 mL), washed with brine (30 mL), and dried over MgSO₄. The organic layer was removed under reduced pressure, and the residue was purified by flash column chromatography (SiO₂, 3:2/EtOAc:Hexanes) to afford compound 9j (147 mg, 64%) as a pale yellow solid: ¹H NMR (500 MHZ, (CD₃)₂SO) δ 10.91 (s, 1H), 9.18 (d, J=1.5 Hz, 1H), 8.92 (d, J=2.5 Hz, 1H), 8.78 (dd, J₁=2.5 Hz, J₂=1.5 Hz, 1H), 8.57 (s, 1H), 6.85 (t, J=1.9 Hz, 1H), 6.73 (d, J=1.9 Hz, 2H); ¹³C NMR (150 MHZ, (CD₃)₂SO) δ 163.1, 151.5, 147.9, 144.4, 143.8, 143.6, 134.4, 117.3, 110.4; HRMS m/z calcd for C₁₁H₈Cl₂N₄O [M+H]⁺: 283.0153; found 283.0159. The purity of the compound was further confirmed by HPLC: Rₜ=15.95 min (95% pure).

### Example 46: Reagents, Fungal Strains, and Culture Conditions

10 mg/mL stock solutions of compounds 1a-9j were prepared in dimethyl sulfoxide (DMSO) and were stored at −20° C. in the dark. The antifungal agent amphotericin B (AmB) was purchased from VWR (Radnor PA, USA). AmB was dissolved in DMSO at a final concentration of 5 mg/mL and was stored at −20° C., protected from light. Candida albicans American Type Culture Collection (ATCC) 10231 (strain A), C. albicans ATCC 64124 (B), and C. albicans ATCC MYA-2876 (C) were obtained from Utah State University. C. albicans ATCC 90819 (D), C. albicans ATCC MYA-2310 (E), C. albicans ATCC MYA-1237 (F), C. albicans ATCC MYA-1003 (G), Candida glabrata ATCC 2001 (H), Candida krusei ATCC 6258 (I), Candida parapsilosis ATCC 22019 (J), Cryptococcus neoformans ATCC MYA-895 (M), Aspergillus terreus ATCC MYA-3633 (N), and Aspergillus flavus ATCC MYA-3631 (0) were obtained from the American Type Culture Collection (ATCC; Manassas, VA, USA). Aspergillus nidulans ATCC 38163 (P) was obtained from University of Kentucky. Clinically derived Aspergillus fumigatus NRRL 163 (Q), A. fumigatus NRRL 5109 (R), and A. fumigatus NRRL 6113 (S) were obtained from Northern Regional Research Laboratory (NRRL). Candida auris 384 (K), C. auris 390 (L), and fungal strains 381-400 (including 10 C. auris (AR Bank #381-390), 3 Candida duobushaemulonii (AR Bank #391, AR Bank #392, and AR Bank #394), 2 Candida haemulonii (AR Bank #393 and AR Bank #395), 1 Kodameae ohmeri (AR Bank #396), 1 C. krusei (AR Bank #397), 1 Candida lusitaniae (AR Bank #398), and 2 Saccharomyces cerevisiae strains (AR Bank #399 and AR Bank #400)) were part of a larger fungal library from the AR Bank maintained by the Centers for Disease Control and Prevention (CDC; Atlanta, GA, USA). Filamentous fungi and yeasts were cultivated at 35° C. in RPMI 1640 medium (with L-glutamine, without sodium bicarbonate, Sigma-Aldrich, St. Louis, MO) buffered to a pH of 7.0 with 0.165 M morpholinepropanesulfonic acid (MOPS) buffer (Sigma-Aldrich). For cytotoxicity assays, HepG2 and the mouse macrophage cell line J774A.1 were obtained from University of Kentucky. The human embryonic kidney cell line, HEK-293, was bought from the ATCC. Mammalian cells were grown in Dulbecco's Modified Eagle's Medium (DMEM) (from VWR) with 10% fetal bovine serum (FBS) (from VWR) and 1% Pen/Strep (from ATCC). Cell lines were cultured at 37° C. with 5% CO₂ and passaged by trypsinization with 0.05% trypsin: 0.53 mM EDTA (from Sigma-Aldrich).

### Example 47: MIC Value Determination by In Vitro Antifungal Assays

The MIC values of compounds 1a-9j against yeast cells (strains A-P and 381-400) were determined in 96-well plates as described in the CLSI documents M27-A3 and M38-A2 with minor modifications.² A single colony of freshly prepared yeast cells was used to inoculate 5 mL of yeast extract peptone dextrose (YPD) broth prior to incubation overnight with shaking at 200 rpm at 35° C. From the actively growing yeast culture, 100 UL were then transferred to 900 μL of RPMI 1640 medium and re-adjusted to achieve OD600 of 0.12 (˜1×10⁶ CFU/mL). The cell suspension was further diluted to achieve 1:100 in RPMI 1640 medium. 100 μL of cells were added to the wells of a 96-well microtiter plates that contained 100 UL of the compound solution to obtain concentrations of 0.06-31.3 μg/mL of compounds 1a-9j or AmB prior to incubation for 48 h at 35° C.

### Example 48: In Vitro Cytotoxicity Assays

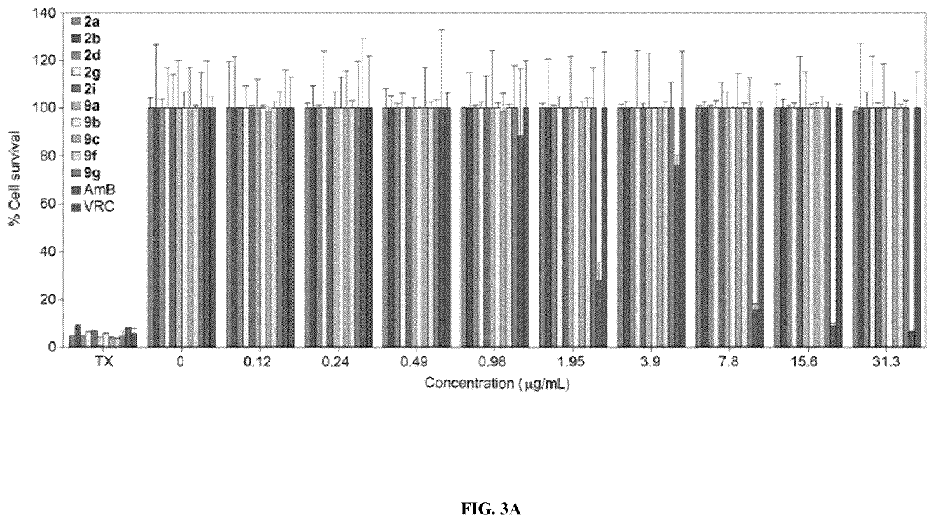

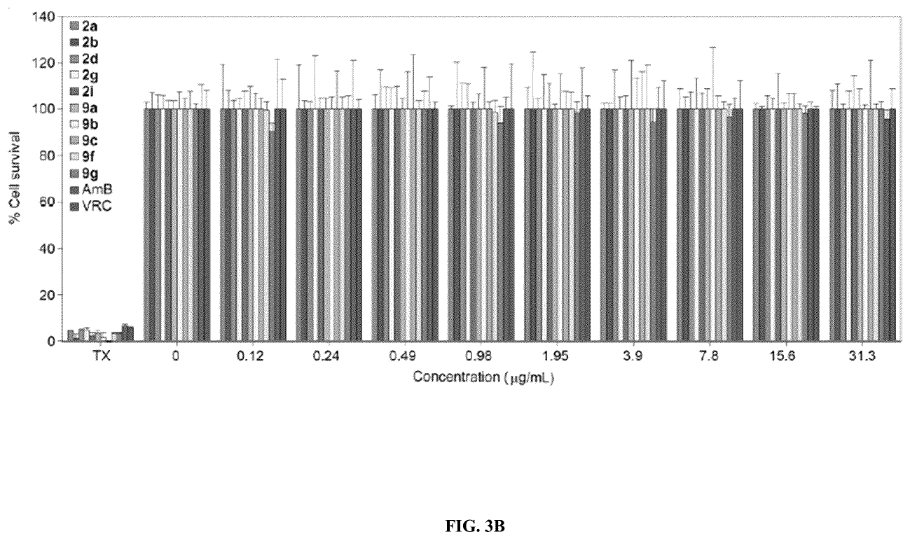

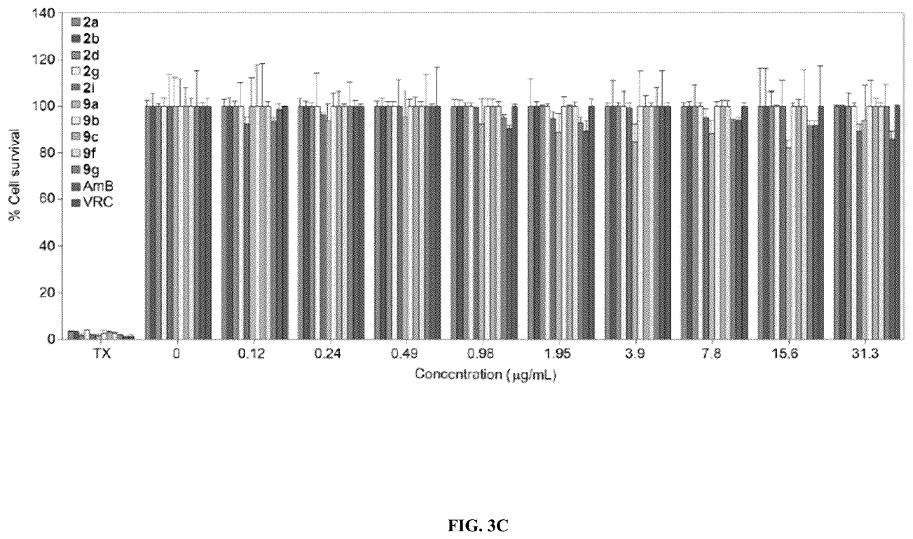

Cytotoxicity assays were performed as previously described with slight modifications.³ HepG2, J774A.1, and HEK-293 cells were first thawed from frozen stocks kept in liquid N₂ and grown in Dulbecco's modified Eagles's medium (DMEM) containing 10% fetal bovine serum (FBS) and 1% Pen/Strep. The adherent cells (HepG2 and HEK-293) were then treated with trypsin using 0.05% trypsin-0.53 mM EDTA and resuspended in fresh DMEM (Note: the trypsin treatment is not needed for J774A.1 cells). Once the cells were 80% confluent, they were transferred to a 96-well microtiter plate at density of 10,000 cells/well for J774A.1, HEK-293, and HepG2. The 96-well plates were incubated 37° C. with 5% CO₂ overnight. The fresh powders of compounds 2a, 2b, 2d, 2g, 2i, 9a, 9b, 9c, 9f, 9g, as well as controls AmB and VRC were prepared as 6.26 mg/mL stock solutions in molecular biology grade DMSO (200× the highest final concentration). The stock solutions were diluted in 1.5 mL eppendorf tubes to achieve concentrations of 6.26-0.024 mg/mL (200×). 5 μL of these 200× compound stock solutions were then added to 495 μL of DMEM in 1.5 mL eppendorf tubes to obtain concentrations of 62.6-0.24 μg/mL (2×). The medium in the 96-well plates containing the cells was aspirated and replaced by fresh DMEM (100 μL). The serially diluted monohydrazides 2a, 2b, 2d, 2g, 2i, 9a, 9b, 9c, 9f, 9g, as well as controls AmB and VRC were added to the 96-well plates to obtain final concentrations of 31.3-0.12 μg/mL. The 96-well plates were further incubated for 24 h at 37° C. with 5% CO₂ overnight. To evaluate cell survival, each well was treated with 10 μL (2 mM) of resazurin sodium salt (Sigma-Aldrich, St. Louis, MO, USA) and was incubated for another 6 h. Metabolically active cells can convert resazurin to the highly fluorescent dye, resorufin, and be detected at ?₅₆₀ excitation and λ₅₉₀ emission using a SpectraMax M5 plate reader (Molecular Devices, San Jose, CA, USA). Triton X-100® (1%, v/v) was used as positive control; the negative control consisted of cells treated with the delivery vehicle (0.05% DMSO), and the blank control was only the culture medium with 0.05% DMSO without cells. The experiments were performed in quadruplicate. The 100% normalized data are reported in FIG. 3.

### Example 49: Time-Kill Assays

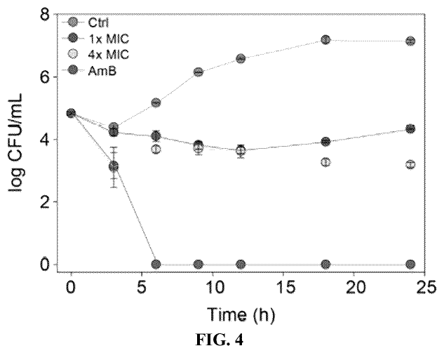

Time-kill assays were used to assess the inhibitory efficiency of compound 2b against C. albicans ATCC 10231 (strain A, FIG. 4). The yeast culture was grown overnight in YPD medium at 35° C. with shaking at 200 rpm. A working stock of fungal cells was made by diluting cultures in RPMI 1640 medium to an OD600 of 0.125. From the working stock, 100 UL of cells were added to 4.9 mL of RPMI 1640 medium in sterile culture tubes, making the starting fungal cell concentration ˜1×10⁵ CFU/mL. Compounds were then added to the fungal cells. The treatment conditions included growth control, AmB (positive control), and compound 2b at 1× and 4×MIC. For C. albicans ATCC 10231 (strain A), the concentration of AmB was 0.98 μg/mL and the concentrations of compound 2b were 0.12 (1×MIC) and 0.48 (4×MIC) μg/mL. The treated fungal cultures were incubated at 35° C. with shaking at 200 rpm for 24 h. Samples were aliquoted from the different treatments at regular time points (0, 3, 6, 9, 12, 18, and 24 h) and plated in duplicate. For each time point, cultures were vortexed, 100 μL of each culture were aspirated, and 10-fold serial dilutions were made in RPMI 1640 medium. From the appropriate dilutions, 100 μL of fungal suspension was spread onto potato dextrose agar (PDA) plates and incubated at 35° C. for 48 h before colony were counted. Experiments were performed in duplicate.

### Example 50: Biofilm Disruption Assays

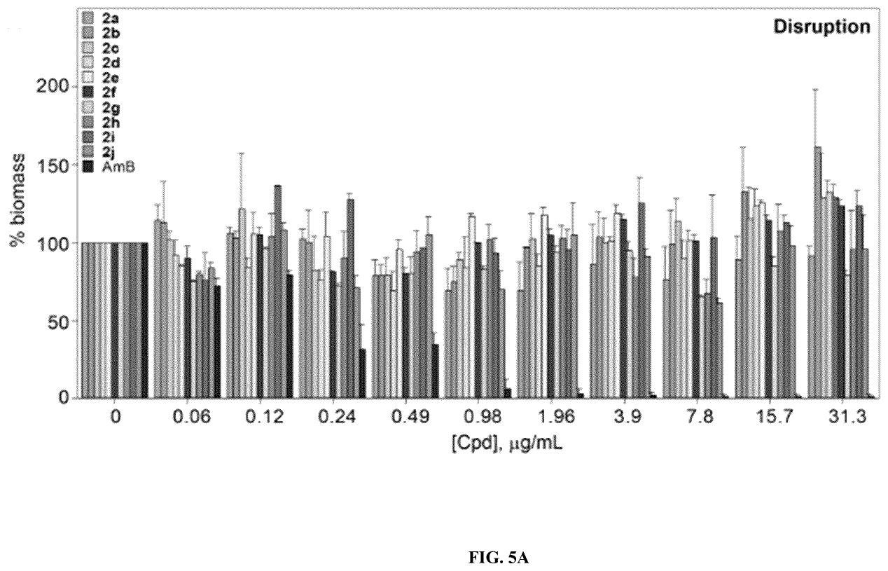

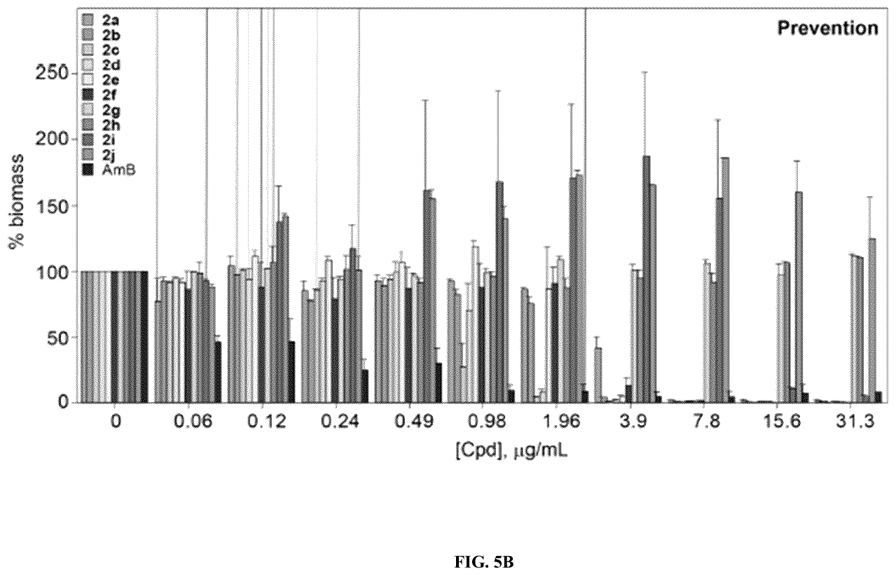

Biofilm disruption assays were performed to assess the effectiveness of compounds 2a-2j against C. albicans ATCC 10231 (strain A, FIG. 5A). AmB was used as a positive control. Biofilm assays were performed in 96-well plates using XTT [2,3-bis(2-methoxy-4-nitro-5-sulfophenyl)-2H-tetrazolium-5-carboxanilide] to measure the viability of the cells in the biofilm, as previously described.⁴ An overnight culture of the yeast cells was grown at 35° C. in YPD medium with shaking at 200 rpm. The overnight culture was diluted in RPMI 1640 medium to an OD600 between 0.12 and 0.15 to make a working stock. The working stock was transferred to 96-well plates in 100 μL aliquots, leaving one column empty for the sterile controls. The plates were incubated at 37° C. for 24 h to allow the biofilm to form. The medium and planktonic cells from the plate were then aspirated. Phosphate buffered saline (PBS) was then used to wash remaining planktonic cells from wells 3 times. After washing, RPMI 1640 medium and the compound were added to the plate, in a similar fashion to that described in the MIC assays. Plates were incubated at 37° C. for 24 h. Finally, the plates were washed 3 times with PBS before adding 100 μL of XTT dye (0.5 mg/mL) with 1 μM menadione. The XTT was prepared by dissolving XTT at 0.5 mg/mL concentration in sterile PBS. After addition of XTT (containing menadione), the plates were incubated for 3 h at 37° C. in the dark. 80 μL of liquid from each well was transferred to new plates. Absorbance at 450 nm was then measured with a SpectraMax M5 plate reader (Molecular Devices, Sunnyvale, CA, USA).

### Example 51: Prevention of Biofilm Formation Assays

Biofilm prevention assays were set up identically to the ordinary MIC experiment, where compounds were diluted, and the fungi added. After overnight growth, the plates were washed as in the biofilm disruption assay and stained and measured in the same fashion. The resulting data are reported in FIG. 5B.

### Example 52: Development of Resistance

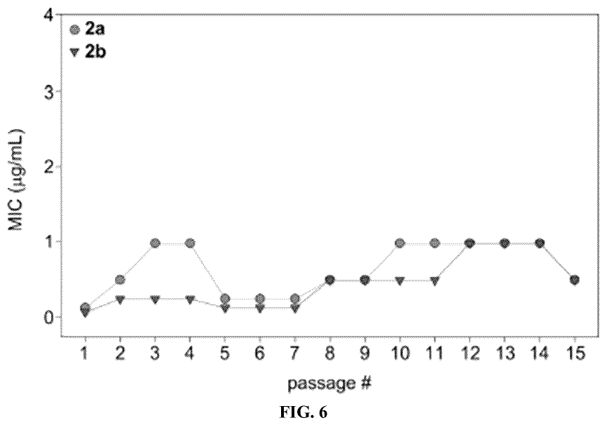

To determine if the fungi would become resistant to the compounds, the change in the MIC of compounds 2a and 2b over 15 serial passages was monitored using C. albicans ATCC 10231 (strain A; FIG. 6). MIC assays were performed as described above. For the serial passages, cells were selected from a well representing ½× the MIC concentration and used to start an overnight culture for the following MIC value determination.

### Example 53: Antifungal Minimum Inhibitory Concentration (MIC) Values

The antifungal activity of 64 monohydrazides (1a-9j) was tested against a panel of seven Candida albicans strains (A-G): ATCC 10231 (A), ATCC 64124 (B), ATCC MYA-2876 (C), ATCC 90819 (D), ATCC MYA-2310 (E), ATCC MYA-1237 (F), and ATCC MYA-1003 (G) (Tables 1 and S1+). The activity of the monohydrazides was also explored against a panel of five non-albicans Candida strains: Candida glabrata ATCC 2001 (H), Candida krusei ATCC 6258 (I), Candida parapsilosis ATCC 22019 (J), Candida auris strain AR Bank #384 (K), and C. auris strain AR Bank #390 (L) (Tables 1A and 1B)

TABLE 1A
MIC values (in μg/mL⁻¹) for most active compounds from 1a-9 and AmB against a variety of Candida albicans,
non-albicans Candida, and non-Candida fungal strains.

| | Fungal strain | | | | | | | | | | | | | | | | | | |
|---|---|---|---|---|---|---|---|---|---|---|---|---|---|---|---|---|---|---|---|
| | Candida albicans | | | | | | | Non-albicans Candida | | | | | Non-Candida | | | | | | |
| Cpd # | A | B | C | D | E | F | G | H | I | J | K | L | M | N | O | P | Q | R | S |
| 1a | 0.24 | 1.95 | 3.9 | 7.8 | 3.9 | 0.98 | 1.95 | 7.8 | 1.95 | 3.9 | 1.95 | 0.24 | 1.95 | 3.9 | >31.3 | 3.9 | >31.3 | >31.3 | >31.3 |
| 1d | 0.49 | 7.8 | 7.8 | 1.95 | 3.9 | 1.95 | 1.95 | 7.8 | 1.95 | 15.6 | 1.95 | 1.95 | 0.49 | 3.9 | >31.3 | 3.9 | >31.3 | >31.3 | >31.3 |
| 1f | 0.98 | 1.95 | 1.95 | 1.95 | 1.95 | 1.95 | 1.95 | 3.9 | 0.98 | 7.8 | 1.95 | 1.95 | 0.98 | 31.3 | >31.3 | 1.95 | >31.3 | >31.3 | >31.3 |
| 2a | ≤0.06 | 0.98 | 0.98 | 0.49 | 1.95 | 0.98 | 0.98 | 1.95 | 0.24 | 1.95 | 1.95 | 0.33 | 0.12 | 1.95 | 7.8 | 3.9 | >31.3 | >31.3 | >31.3 |
| 2b | 0.12 | 0.98 | 0.98 | 0.49 | 0.98 | 0.98 | 0.49 | 1.95 | 0.49 | 1.95 | 0.98 | 0.98 | 0.12 | 0.98 | >31.3 | 0.24 | >31.3 | >31.3 | >31.3 |
| 2c | 0.24 | 0.98 | 0.98 | 1.95 | 1.95 | 1.95 | 0.98 | 13.6 | 0.49 | 3.9 | 0.98 | 0.98 | 0.24 | 3.9 | >31.3 | 1.95 | 7.8 | 3.9 | >31.3 |
| 2d | 0.12 | 0.98 | 3.9 | 0.98 | 1.95 | 1.95 | 0.49 | 1.95 | 0.12 | 1.95 | 3.9 | 0.98 | 0.24 | 1.95 | 31.3 | 0.49 | 31.3 | 7.8 | 15.6 |
| 2e | 0.12 | 0.98 | 1.95 | 0.24 | 0.98 | 0.98 | 0.98 | 1.95 | 1.95 | 3.9 | 1.95 | 1.95 | 0.49 | 3.9 | 31.3 | 0.49 | >31.3 | 7.8 | 15.6 |
| 2f | 0.12 | 0.98 | 1.95 | 0.49 | 0.98 | 0.98 | 0.98 | 0.98 | 0.49 | 3.9 | 3.9 | 0.12 | 0.49 | 31.3 | >31.3 | 0.49 | >31.3 | >31.3 | 31.3 |
| 2g | 0.12 | 1.95 | 0.98 | 0.98 | 31.3 | 0.49 | 0.49 | 0.98 | 0.49 | 1.95 | 0.49 | 0.12 | 0.24 | 0.98 | 15.6 | 0.49 | >31.3 | 15.6 | 31.3 |
| 2h | 0.12 | 1.95 | 3.9 | 0.98 | 1.95 | 1.95 | 0.98 | 3.9 | 0.24 | 3.9 | 0.24 | 0.98 | 0.49 | 0.98 | 3.9 | 0.49 | >31.3 | 15.6 | >31.3 |
| 2i | ≤0.06 | 0.98 | 0.98 | 0.98 | 0.98 | 1.95 | 0.49 | 7.8 | 0.24 | 1.95 | 0.24 | 0.49 | 0.24 | 0.98 | 1.95 | 0.49 | ≤0.06 | ≤0.06 | 31.3 |
| 2j | 0.24 | 1.95 | 1.95 | 0.98 | 1.95 | 3.9 | 1.95 | 7.8 | 0.98 | 1.95 | 7.8 | 1.95 | 0.49 | 1.95 | >31.3 | 0.49 | >31.3 | >31.3 | >31.3 |
| 3a | 0.49 | 0.49 | 3.9 | 3.9 | 3.9 | 3.9 | 1.95 | 31.3 | 0.24 | 1.95 | 1.95 | 0.98 | ≤0.06 | 1.95 | 7.8 | 0.98 | >31.3 | >31.3 | >31.3 |
| 3c | 0.24 | 0.95 | 0.49 | 3.9 | 1.95 | 1.95 | 0.98 | [symbol] | 0.24 | 3.9 | 1.95 | 0.98 | 0.24 | 3.9 | >31.3 | 1.95 | >31.3 | >31.3 | >31.3 |
| 3f | 0.24 | 0.98 | 0.98 | 0.98 | 0.98 | 0.49 | 0.49 | 1.95 | 0.24 | 3.9 | 3.9 | 0.49 | ≤0.06 | 31.3 | >31.3 | 1.95 | >31.3 | >31.3 | >31.3 |
| 4a | 0.49 | 0.49 | 1.95 | 3.9 | 3.9 | 3.9 | 0.98 | >31.3 | 0.24 | 7.8 | 1.95 | 1.95 | 0.06 | 0.98 | 15.6 | 1.95 | >31.3 | >31.3 | >31.3 |
| 4e | 0.98 | 3.9 | 0.98 | 1.95 | 3.9 | 0.98 | 3.9 | 3.9 | 0.24 | 1.95 | 0.98 | 1.95 | 0.49 | 1.95 | >31.3 | 3.9 | >31.3 | 15.6 | >31.3 |
| 4c | 0.98 | 0.98 | 0.49 | 3.9 | 1.95 | 1.95 | 0.98 | 15.6 | 0.24 | 3.9 | 1.95 | 0.98 | 0.49 | 3.9 | >31.3 | 1.95 | 15.6 | 7.8 | >31.3 |
| 5d | 0.24 | 0.98 | 1.95 | 1.95 | 3.9 | 1.95 | 0.98 | 15.6 | 0.49 | 3.9 | 1.95 | 0.98 | 0.49 | 1.95 | 7.8 | 0.98 | 31.3 | 15.6 | 15.6 |
| 5f | 0.24 | 1.95 | 0.98 | 1.95 | 0.98 | 0.98 | 0.98 | 0.98 | 0.49 | 7.8 | 1.95 | 1.95 | 0.24 | 15.6 | >31.3 | 1.95 | >31.3 | >31.3 | 15.6 |
| 6g | 1.95 | 3.9 | 0.49 | 1.95 | 7.8 | 0.98 | 3.9 | 7.8 | 0.24 | 0.98 | 0.98 | 1.95 | 0.98 | 1.95 | >31.3 | 3.9 | >31.3 | 31.3 | >31.3 |
| 7a | 0.24 | 0.98 | 1.95 | 3.9 | 1.95 | 3.9 | 1.95 | 31.3 | 0.24 | 1.95 | 1.95 | 1.95 | ≤0.06 | 3.9 | >31.3 | 0.98 | >31.3 | >31.3 | >31.3 |
| 7d | 0.98 | 1.95 | 1.95 | 1.95 | 3.9 | 1.95 | 0.98 | 15.6 | 0.49 | 3.9 | 1.95 | 0.98 | 0.49 | 3.9 | 7.8 | 0.49 | >31.3 | >31.3 | >31.3 |
| 7f | 1.95 | 1.95 | 1.95 | 1.95 | 1.95 | 0.98 | 0.98 | 7.8 | 0.98 | 7.8 | 3.9 | 0.98 | 1.95 | 31.3 | >31.3 | 1.95 | 31.3 | 31.3 | 31.3 |
| 8a | 0.98 | 0.98 | 1.95 | 3.9 | 1.95 | 1.95 | 0.98 | 15.6 | 0.49 | 7.8 | 1.95 | 1.95 | ≤0.06 | 3.9 | >31.3 | 1.95 | >31.3 | >31.3 | >31.3 |
| 8d | 0.24 | 0.98 | 0.98 | 0.98 | 0.98 | 0.98 | 0.49 | 15.6 | 0.24 | 7.8 | 1.95 | 0.98 | 0.49 | 1.95 | 7.8 | 0.49 | 31.3 | 15.6 | >31.3 |
| 8f | 1.95 | 1.95 | 1.95 | 1.95 | 1.95 | 0.98 | 0.98 | 7.8 | 0.98 | 7.8 | 3.9 | 0.98 | 0.98 | 31.3 | >31.3 | 1.95 | 31.3 | 15.6 | 31.3 |
| 9a | 0.24 | 1.95 | 3.9 | 0.98 | 7.8 | 0.98 | 0.98 | 7.8 | 0.49 | 3.9 | 0.98 | 0.98 | 0.12 | 0.98 | 15.6 | 0.98 | >31.3 | >31.3 | >31.3 |
| 9b | 0.24 | 1.95 | 1.95 | 3.9 | 7.8 | 0.98 | 0.98 | 3.9 | 0.24 | 1.95 | 1.95 | 0.98 | 0.24 | 0.98 | >31.3 | 0.98 | >31.3 | >31.3 | >31.3 |
| 9c | 0.49 | 0.98 | 1.95 | 1.95 | 1.95 | 1.95 | 0.98 | 7.8 | 0.49 | 1.95 | 1.95 | 0.98 | 0.49 | 1.95 | >31.3 | 1.95 | 15.6 | 7.8 | >31.3 |
| 9f | 0.49 | 0.98 | 1.95 | 0.98 | 1.95 | 0.98 | 1.95 | 0.98 | 0.98 | 3.9 | 0.49 | 0.49 | 0.98 | >31.3 | >31.3 | 0.98 | 31.3 | 31.3 | 31.3 |
| 9g | 0.24 | 0.98 | 0.98 | 0.98 | 3.9 | 0.49 | 0.49 | 0.98 | 0.24 | 3.9 | 3.9 | 0.98 | 0.24 | 0.98 | 7.8 | 0.49 | >31.3 | >31.3 | >31.3 |
| AmB | 1.95 | 1.95 | 1.95 | 0.98 | 0.98 | 1.95 | 0.98 | 0.98 | 0.98 | 0.98 | 1.95 | 1.95 | 7.8 | 3.9 | 31.3 | 15.6 | 0.98 | 0.98 | 1.95 |

Strains: A = C. albicans ATCC 10231, B = C. albicans ATCC 64124, C. albicans ATCC MYA-2876(S), D = C. albicans ATCC 90819(R), E = C. albicans ATCC MYA-2310(8), F = C. albicans ATCC MYA-1237(R), G = C. albicans ATCC MYA-1063(R), H = C. glabrata ATCC 2004, I = C. krusei ATCC 6258, J = C. parapsilosis ATCC 22019, K = C. auris AR Bank # 0384, L = C. auris AR Bank # 0390, M = C. neoformans ATCC MYA-895, N = A. terreus ATCC MYA-3633, O = A. flavus ATCC MYA-3631, P = A. nidulans ATCC 38163, Q = A. fumigatus NRR1, 163, R = A. fumigatus NRRI, 5109, and S = A. fumigatus NRRI, 6113,
NOTE:
Here, the (S) and (R) indicate that ATCC reports these strains to be susceptible (S) and resistant (R) to [symbol] (ITC) and [symbol] (FLC). [symbol]
indicates data missing or illegible when filed

Finally, the activity of these monohydrazides was explored against four non-Candida strains: Cryptococcus neoformans ATCC MYA-895 (M), Aspergillus terreus ATCC MYA-3633 (N), Aspergillus flavus ATCC MYA-3631 (O), Aspergillus nidulans ATCC 38163 (P), Aspergillus fumigatus NRRL 163 (Q), A. fumigatus NRRL 5109 (R), and A. fumigatus NRRL 6113 (S) (Tables 1A and 1B). The double dilution method was used with concentrations ranging from 0.06 to 31.3 j·tg mL⁻¹ for monohydrazides 1a-9j, and the commercially available positive antifungal control amphotericin B (AmB). Herein, based on the MIC values (i.e., no visible growth), antifungal activity was defined as excellent (≤1.95 j·tg mL⁻¹), good (3.9-7.8 j·tg mL⁻¹), or poor (≥15.6 j·tg mL⁻¹). Antifungal data for the most active compounds are presented in Table 1A and the remaining are available in Table 1B.

TABLE 1B
MIC values (in μg/mL⁻¹) for remaining compounds from 1a-9 and AmB against a variety of Candida albicans,
non-albicans Candida, and non-Candida fungal strains.

| | Fungal strain | | | | | | | | | | | | | | | |
|---|---|---|---|---|---|---|---|---|---|---|---|---|---|---|---|---|
| | Candida albicans | | | | | | | non-albicans Candida | | | | | non-Candida | | | |
| Cpd # | A | B | C | D | E | F | G | H | I | J | K | L | M | N | O | P |
| 1b | 1.95 | 7.8 | 7.8 | 15.6 | 7.8 | 3.9 | 3.9 | >31.3 | 0.98 | 3.9 | 13.6 | 7.8 | 1.95 | 7.8 | >31.3 | 3.9 |
| 1c | 0.98 | 1.95 | 3.9 | 3.9 | 3.9 | 3.9 | 1.95 | 31.3 | 0.98 | 7.8 | 1.95 | 1.95 | 0.49 | 3.9 | >31.3 | 3.9 |
| 1e | 1.95 | 15.6 | 15.6 | 3.9 | 7.8 | 1.95 | 7.8 | 3.9 | 15.6 | 31.3 | 7.8 | >31.3 | 1.95 | 31.3 | >31.3 | 31.3 |
| 1g | 1.95 | 1.95 | 3.9 | 3.9 | >31.3 | 1.95 | 0.98 | 1.95 | 7.8 | 7.8 | 15.6 | 7.8 | 0.49 | 31.3 | >31.3 | 1.95 |
| 1h | 3.9 | 15.6 | 31.3 | 31.3 | 31.3 | 31.3 | 31.3 | >31.3 | 3.9 | 31.3 | 31.3 | 15.6 | 0.98 | >31.3 | 15.6 | 3.9 |
| 1i | 3.9 | 15.6 | 15.6 | 15.6 | 15.6 | 7.8 | 15.6 | >31.3 | 1.95 | 15.6 | 15.6 | 15.6 | 0.49 | >31.3 | >31.3 | 7.8 |
| 1j | 3.9 | >31.3 | >31.3 | >31.3 | >31.3 | 15.6 | 7.8 | >31.3 | 1.95 | >31.3 | >31.3 | >31.3 | 7.8 | >31.3 | >31.3 | 15.6 |
| 3d | 0.49 | 1.95 | 3.9 | 1.95 | 3.9 | 1.95 | 1.95 | 15.6 | 0.49 | 3.9 | 3.9 | 0.98 | 0.98 | 3.9 | 15.6 | 0.98 |
| 3e | 1.95 | 1.95 | 1.95 | 3.9 | 1.95 | 1.95 | 0.98 | 7.8 | 0.98 | 31.3 | 7.8 | 7.8 | 3.9 | >31.3 | >31.3 | 15.6 |
| 3g | 0.49 | 1.95 | 1.95 | 1.95 | 1.95 | 1.95 | 0.98 | 31.3 | 0.98 | 31.3 | 7.8 | 3.9 | 0.98 | 3.9 | 15.6 | 0.98 |
| 4d | 1.95 | 7.8 | 7.8 | 7.8 | 7.8 | 7.8 | 1.9 | >31.3 | 0.98 | 7.8 | 7.8 | 3.9 | 0.98 | 7.8 | 15.6 | 1.95 |
| 4f | 3.9 | 3.9 | 1.95 | 3.9 | 3.9 | 0.98 | 1.95 | 1.95 | 0.98 | 3.9 | 1.95 | 1.95 | 1.95 | 7.8 | >31.3 | 15.6 |
| 4g | 3.9 | 15.6 | 0.98 | 3.9 | >31.3 | 1.95 | 15.6 | 7.8 | 0.49 | 3.9 | 3.9 | 31.3 | 1.95 | 3.9 | >31.3 | 3.9 |
| 5a | 0.49 | 0.49 | 3.9 | 7.8 | 1.95 | 1.95 | 1.95 | 31.3 | 0.24 | 7.8 | 1.95 | 3.9 | ≤0.06 | 1.95 | >31.3 | 0.98 |
| 5e | 1.95 | 1.95 | 1.95 | 3.9 | 1.95 | 0.98 | 0.98 | 3.9 | 1.95 | 15.6 | 3.9 | 7.8 | 1.95 | 31.3 | >31.3 | 7.8 |
| 5g | 0.98 | 1.95 | 1.95 | 3.9 | 1.95 | 1.95 | 0.98 | 31.3 | 0.98 | 31.3 | 7.8 | 3.9 | 0.49 | 3.9 | 31.3 | 0.49 |
| 6a | 0.49 | 1.95 | 1.95 | 7.8 | 3.9 | 3.9 | 3.9 | >31.3 | 0.49 | 1.95 | 3.9 | 1.95 | ≤0.06 | 3.9 | 31.3 | 1.95 |
| 6d | 0.98 | 1.95 | 3.9 | 3.9 | 3.9 | 3.9 | 1.95 | >31.3 | 0.49 | 7.8 | 3.9 | 0.98 | 0.98 | 3.9 | 15.6 | 0.98 |
| 6f | 1.95 | 3.9 | 1.95 | 3.9 | 3.9 | 0.98 | 3.9 | 1.95 | 0.98 | 3.9 | 1.95 | 1.95 | 1.95 | 7.8 | >31.3 | 3.9 |
| 6g | 3.9 | 15.6 | 0.98 | 3.9 | 31.3 | 1.95 | 15.6 | 15.6 | 0.98 | 7.8 | 3.9 | 31.3 | 0.49 | 7.8 | >31.3 | 1.95 |
| 7c | 0.98 | 1.95 | 3.9 | 7.8 | 3.9 | 3.9 | 1.95 | 15.6 | 15.6 | 15.6 | 7.8 | 15.6 | 15.6 | 31.3 | >31.3 | 15.6 |
| 7e | 3.9 | 3.9 | 3.9 | 7.8 | 1.95 | 0.49 | 1.95 | 3.9 | 1.95 | 15.6 | 3.9 | 7.8 | 1.95 | >31.3 | >31.3 | 7.8 |
| 7g | 0.98 | 3.9 | 3.9 | 3.9 | 3.9 | 3.9 | 1.95 | >31.3 | 0.98 | 31.3 | 7.8 | 1.95 | 0.98 | 7.8 | >31.3 | 0.98 |
| 8c | 0.98 | 1.95 | 3.9 | 7.8 | 3.9 | 1.95 | 1.95 | 15.6 | 15.6 | 7.8 | 15.6 | 15.6 | 7.8 | 31.3 | >31.3 | 7.8 |
| 8e | 3.9 | 7.8 | 3.9 | 7.8 | 3.9 | >31.3 | 3.9 | 3.9 | 3.9 | 15.6 | 7.8 | 7.8 | 1.95 | >31.3 | >31.3 | 7.8 |
| 8g | 0.98 | 1.95 | 1.95 | 3.9 | 1.95 | 1.95 | 0.98 | 15.6 | 0.49 | 15.6 | 3.9 | 1.95 | 0.49 | 31.3 | >31.3 | 0.49 |
| 9d | 0.24 | 1.98 | 7.8 | 0.98 | 7.8 | 0.98 | 1.95 | 3.9 | 0.49 | 3.9 | 0.49 | 0.98 | 0.24 | 1.95 | 15.6 | 0.49 |
| 9e | 0.98 | 1.95 | 3.9 | 0.98 | 1.95 | 0.98 | 1.95 | 1.95 | 3.9 | 7.8 | 1.95 | 3.9 | 0.98 | 7.8 | >31.3 | 1.95 |
| 9h | 0.24 | 7.8 | 3.9 | 3.9 | >31.3 | 3.9 | 1.95 | 15.6 | 0.49 | 7.8 | 0.49 | 1.95 | 0.49 | 1.95 | >31.3 | 1.95 |
| 9i | 0.24 | 3.9 | 7.8 | 3.9 | 15.6 | 1.95 | 1.95 | 7.8 | 0.49 | 3.9 | 0.49 | 1.95 | 0.24 | 1.95 | 3.9 | 1.95 |
| 9j | 0.49 | 3.9 | 3.9 | 1.95 | 7.8 | 1.95 | 3.9 | 15.6 | 0.98 | 3.9 | 15.6 | 7.8 | 0.49 | 3.9 | >31.3 | 7.8 |
| AmB | 1.95 | 1.95 | 1.95 | 0.98 | 0.98 | 1.95 | 1.95 | 0.98 | 0.98 | 0.98 | 1.95 | 1.95 | 7.8 | 3.9 | 31.3 | 15.6 |

Strains: A = C. albicans ATCC 10231, B = C. albicans ATCC 64124, C. albicans ATCC MYA-2876(S), D = C. albicans ATCC 90819(R), E = C. albicans ATCC MYA-2310(8), F = C. albicans ATCC MYA-1237(R), G = C. albicans ATCC MYA-1063(R), H = C. glabrata ATCC 2004, I = C. krusei ATCC 6258, J = C. parapsilosis ATCC 22019, K = C. auris AR Bank # 0384, L = C. auris AR Bank # 0390, M = C. neoformans ATCC MYA-895, N = A. terreus ATCC MYA-3633, O = A. flavus ATCC MYA-3631, P = A. nidulans ATCC 38163,
NOTE:
Here, the (S) and (R) indicate that ATCC reports these strains to be susceptible (S) and resistant (R) to [symbol] (ITC) and [symbol] (FLC). [symbol]
indicates data missing or illegible when filed

From the data reported in Tables 1A and 1B, compounds from series 2 and series 9 performed better compared to those in the other seven series of compounds against the sixteen strains (A-P) tested. A detailed analysis of the nine series (i.e., series 1-9) led to the following conclusions. In the case of monohydrazides 1a-1j (i.e., R1=2,4-diF, X═C, Y═C; FIG. 1), compounds 1h, 1i, and 1j generally displayed poor activity against strains A-P, with the exception of the following combinations; 1h (MIC=0.98 j·tg mL⁻¹ against strain M), 1i (MIC=1.95 j·tg mL⁻¹ and 0.49 j·tg mL⁻¹ against strains I and M, respectively), and 1j (MIC=1.95 j·tg mL⁻¹ against strain I). Among series 1, compounds 1f, 1a, 1d, and 1c performed better with excellent activity (0.24-1.95 j·tg mL⁻¹) against twelve strains (A-G, I, K-M, and P), nine strains (A, B, F, G, I, and K-N), eight strains (A, D, F, G, I, and K-M), and seven strains (A, B, G, I, and K-M), respectively. Monohydrazides 2a-2j (i.e., R1=H, X═N, Y═C; FIG. 1) displayed excellent to good activity (0.06-7.8 j·tg mL⁻¹) against all fungal strains tested with the exception of compounds 2b, 2c, 2d, 2c, 2f, 2g, and 2j against strains O, H (2c only), N (2f only), and E (2g only). All the compounds from series 3 (3a, 3c, 3d, 3c, 3f, and 3g) (i.e., R1=5-F, X═N, Y═C; FIG. 1) displayed excellent to good activity (0.06-7.8 j·tg mL⁻¹) against most of the strains A-P. However, some compounds from series 3 displayed poor activity (15.6->31.3 j·tg mL⁻¹). Those include compounds 3a, 3c, 3d, and 3g against strain H, compounds 3e and 3f against strain N, compounds 3e and 3g against strain J, compound 3e against strain P, and all “3”-compounds except 3a against strain O. Among series 3, compounds 3f, 3c, 3a, and 3g were the best in terms of their activity. In the case of monohydrazides from series 4 (i.e., R1=5-Br, X═N, Y═C; FIG. 1), compounds 4a, 4c, 4d, and 4f displayed excellent to good activity against strains A-G and I-N (0.24-7.8 j·tg mL⁻¹). However, compound 4g performed below par with poor activity against strains B, E, G, L, and O. For series 5 (i.e., R1=6-F, X═N, Y═C; FIG. 1), compounds 5a, 5c, 5d, and 5f exhibited excellent to good activity (0.06-7.8 j·tg mL⁻¹) against 14 to 15 (out of 16) of the fungal strains tested, with the exception of compounds 5a, 5c, 5d, and 5f against either strains H, N, and/or O. Compounds 5e and 5g displayed excellent to good activity against strains A-I, K-M, and P (0.98-7.8 j·tg mL⁻¹), and strains A-G, I, K-N, and P (0.49-7.8 j·tg mL⁻¹), respectively. Amongst monohydrazides 6a, 6c, 6d, 6f, and 6g (i.e., R1=6-Br, X═N, Y═C; FIG. 1), compounds 6a and 6d exhibited excellent to good activity (0.06-7.8 j·tg mL⁻¹) against all 16 strains tested with the exception of strains H and O. Compounds 6c and 6f displayed excellent to good activity (0.24-7.8 j·tg mL⁻¹) against all 16 strains tested with the exception of strain O. On the other hand, compound 6g performed poorly against strains B, E, G, H, L, and O. For compounds from series 7 (i.e., R1=3,5-diF, X═N, Y═C; FIG. 1), compounds 7a, 7d, and 7f performed better than other members of this series. Compounds 7a, 7d, and 7f exhibited excellent to good activity (0.06-7.8 j·tg mL⁻¹) against all 16 strains tested, with the exception of compounds 7a, 7d, and 7f against strains H and O, strain H, and strains N and O, respectively. Compounds 7e, and 7g displayed excellent activity (0.49-1.98 j·tg mL⁻¹) against strains E-G, I, and M, and strains A, G, I, L, M, and P, respectively. For series 8 (i.e., R1=3,6-diF, X═N, Y═C; FIG. 1), compound 8d exhibited excellent to good activity (0.24-7.8 j·tg mL⁻¹) against all the strains tested, with the exception of strain H. Compounds 8a, 8f, and 8g displayed excellent to good activity (0.06-7.8 j·tg mL⁻¹) against strains A-G, I-N, and P, strains A-M, and P, and strains A-G, I, K-M, and P, respectively. Amongst series 8, compounds 8c and 8e performed subpar when compared to other compounds in the same series. Finally, in the case of series 9 (i.e., R1≡H, X═N, Y═N; FIG. 1), compound 9c exhibited excellent activity (0.49-1.95 j·tg mL⁻¹) against 14 strains A-G, I-N, and P. Among the remaining compounds in series 9, compounds 9f, 9g, 9b, 9a, 9d, 9c, and 9i displayed excellent to good activity (0.12-7.8 j·tg mL⁻¹) against all 16 strains tested with the exception of compounds 9f, 9b, 9a, 9d, 9c, and 9i against strains E (9i only was inactive), N (9f only was inactive), and O. Additionally, the activity of compounds 1a, 1d, 1f, 2a-2j, 3a, 3c, 3f, 4a, 4c, 5c, 5d, 5f, 6c, 7a, 7d, 7f, 8a, 8d, 8f, 9a-9c, 9f, and 9g was explored against clinically derived A. fumigatus strains Q, R, and S. A few compounds (2c, 2d, 2c, 5c, and 9c) exhibited good activity against strains Q and R. The highest activity against strain S was 15.6 j·tg mL⁻¹ for compounds 2d, 2e, 5e and 5f. In summary, compounds 1f, 2a, 2b, 2c, 2d, 2c, 2f, 2g, 2 h, 2i, 2j, 3f, 5d, 5f, 8d, 9b, 9c, 9f, and 9g exhibited a comparable or superior activity to that of the FDA-approved antifungal agent AmB against most of the tested strains, with the exception of strains Q, R and S, against which AmB was 4-8-fold more potent than the most active monohydrazides. From all of the observations made for compounds 1a-9j, series 2 performed the best followed by series 9.

Based on the promising antifungal activities observed in Tables 1A and 1B, the entire series 2 was selected, and some of the representative compounds from series 1 and 3-9 for further testing against a panel of ten C. auris strains (AR Bank #381-390) (Table 2A and 2B). Using the same concentration range as above (0.06 to 31.3 j·tg mL⁻¹) for all of the selected monohydrazides and using AmB as a positive control, MIC values were determined. Monohydrazides 2a, 2b, 2g, 2 h, 2i, 5d, 8d, 9b, and 9f displayed excellent activity (0.06-1.95 j·tg mL⁻¹) against all ten C. auris strains tested.

TABLE 2A
MIC values (in μg/mL⁻¹) for compounds selected as well as AmB against a variety of non-albicans Candida and
non-Candida fungal strains.

| Cpd | | | | 384 | | | | | | 390 | | | | | | | | | | |
|---|---|---|---|---|---|---|---|---|---|---|---|---|---|---|---|---|---|---|---|---|
| # | 381 | 382 | 383 | (K) | 385 | 386 | 387 | 388 | 389 | (L) | 391 | 392 | 393 | 394 | 395 | 396 | 397 | 398 | 399 | 400 |
| 2a | 0.49 | 0.98 | 1.95 | 1.95 | 0.98 | 0.49 | 0.49 | 0.49 | 0.12 | 0.24 | 0.49 | 0.24 | 0.12 | 0.49 | ≤0.06 | 0.24 | 0.24 | 0.98 | 1.95 | [symbol] |
| 2b | 0.98 | 0.49 | 0.49 | 0.98 | 0.49 | 0.24 | 0.49 | 0.49 | 0.49 | 0.49 | 0.24 | 0.24 | 0.12 | 0.12 | ≤0.06 | 0.12 | 0.12 | 0.98 | 0.98 | [symbol] |
| 2c | 3.9 | 0.98 | 0.98 | 0.98 | 3.9 | 0.49 | 0.49 | 3.9 | 0.49 | 0.98 | 1.95 | 1.95 | 0.49 | 0.98 | 0.49 | 0.49 | 0.49 | 1.95 | 7.8 | 7.8 |
| 2d | 0.98 | 1.95 | 1.95 | 3.9 | 0.98 | 0.49 | 0.98 | 0.98 | 0.49 | 0.98 | 0.49 | 0.49 | 0.24 | 0.24 | 0.12 | 0.24 | 0.24 | 1.95 | 3.9 | [symbol] |
| 2e | 1.95 | 3.9 | 1.95 | 1.95 | 1.95 | 0.98 | 1.95 | 1.95 | 0.98 | 1.95 | 0.98 | 0.98 | 0.49 | 0.49 | 0.12 | 0.24 | 0.24 | 0.98 | 0.98 | [symbol] |
| 2f | 1.95 | 1.95 | 1.95 | 3.9 | 0.98 | 0.24 | 0.49 | 0.49 | 0.24 | 0.12 | 0.98 | 0.98 | 0.49 | 0.98 | 0.49 | 0.49 | 0.49 | 0.98 | 1.95 | [symbol] |
| 2g | 0.49 | 0.49 | 0.49 | 0.49 | 0.49 | 0.49 | 0.12 | 0.12 | 0.24 | 0.12 | 0.12 | 0.49 | 0.49 | 3.9 | 0.24 | 0.98 | ≤0.06 | 1.95 | 0.49 | [symbol] |
| 2h | 1.95 | 0.24 | 0.49 | 1.95 | 0.49 | 0.12 | 0.49 | 0.49 | 0.49 | 0.98 | 0.49 | 0.24 | 0.98 | 0.24 | 0.24 | 0.12 | 0.24 | 0.49 | 1.95 | [symbol] |
| 2i | 0.98 | 0.12 | 0.24 | 0.98 | 0.24 | ≤0.06 | 0.24 | 0.24 | 0.24 | 0.49 | 0.24 | 0.49 | 0.49 | 0.24 | 0.24 | 0.12 | 0.12 | 0.49 | 1.95 | [symbol] |
| 2j | 3.9 | 0.98 | 1.95 | 7.8 | 1.95 | 1.98 | 0.98 | 3.9 | 1.95 | 3.9 | 0.98 | 0.98 | 0.24 | 0.49 | 0.24 | 0.49 | 0.49 | 3.9 | 3.9 | 3.9 |
| 9b | 0.49 | 0.12 | 0.24 | 0.98 | 0.24 | ≤0.06 | 0.24 | 0.24 | 0.24 | 0.49 | 0.24 | 0.12 | 0.24 | 0.12 | 0.24 | 0.12 | 0.12 | 0.49 | 0.98 | [symbol] |
| 9c | 1.95 | 0.98 | 1.95 | 1.95 | 7.8 | 0.49 | 0.98 | 3.9 | 0.98 | 0.98 | [symbol] | 0.98 | 0.49 | 1.95 | 0.49 | 0.49 | 0.49 | 1.95 | 7.8 | 3.9 |
| 9e | 7.8 | 13.6 | 3.9 | 1.95 | 31.3 | 7.8 | 13.6 | 7.8 | 7.8 | 3.9 | 1.95 | 1.95 | 1.95 | 1.95 | 3.9 | 1.95 | 1.9 | 3.9 | 3.9 | 3.9 |
| 9f | 1.95 | 0.98 | 0.98 | 0.98 | 0.98 | 0.49 | 1.95 | 0.98 | 0.98 | 0.98 | 0.49 | 0.49 | 0.24 | 0.49 | 0.49 | 0.98 | 0.49 | 0.98 | 0.98 | [symbol] |
| 9g | 3.9 | 1.95 | 0.98 | 3.9 | 0.98 | 0.49 | 0.49 | 0.49 | 0.49 | 0.98 | 0.49 | 0.24 | 0.24 | 7.8 | 1.95 | 0.98 | 1.95 | 3.9 | 7.8 | [symbol] |
| AmB | 0.98 | 0.98 | 1.95 | 1.95 | 1.95 | 0.98 | 0.98 | 1.95 | 1.95 | 1.95 | 7.8 | 7.8 | 13.6 | 0.98 | 3.9 | 1.95 | 0.49 | 3.9 | 15.6 | [symbol] |

Strains: 381-390 = Candida auris, 391, 392, and 394 = Candida [symbol] , 393 and 395 = Candida [symbol] , 396 = [symbol] [symbol] 397 = Candida [symbol] , 398 = Candida [symbol] , 399 and 400 = Saccharomyces cerevisiae. [symbol]
indicates data missing or illegible when filed

TABLE 2B
MIC values (in μg/mL⁻¹) for compounds selected as well as AmB against a variety of Candida auris fungal strains.

| | C. auris fungal strain (AR Bank #) | | | | | | | | | |
|---|---|---|---|---|---|---|---|---|---|---|
| Cpd # | 381 | 382 | 383 | 384 (K) | 385 | 386 | 387 | 388 | 389 | 390 (L) |
| 1b | 3.9 | 3.9 | 15.6 | 7.8 | 3.9 | 7.8 | 7.8 | 3.9 | 3.9 | 7.8 |
| 1j | >31.3 | 15.6 | >31.3 | 15.6 | 31.3 | >31.3 | >31.3 | >31.3 | >31.3 | >31.3 |
| 3c | 0.98 | 0.49 | 0.98 | 0.49 | 0.49 | 0.98 | 0.98 | 0.98 | 0.98 | 3.9 |
| 3d | 1.95 | 0.98 | 1.95 | 0.98 | 0.98 | 1.95 | 0.98 | 1.95 | 1.95 | 3.9 |
| 3e | 7.8 | 7.8 | 3.9 | 7.8 | 1.95 | 7.8 | 7.8 | 7.8 | 7.8 | 7.8 |
| 3g | 1.95 | 1.95 | 1.95 | 0.98 | 0.49 | 1.95 | 0.98 | 1.95 | 1.95 | 3.9 |
| 4d | 1.95 | 1.95 | 7.8 | 1.95 | 1.95 | 3.9 | 3.9 | 1.95 | 3.9 | 7.8 |
| 5c | 0.98 | 0.49 | 1.95 | 0.49 | 0.98 | 0.98 | 0.49 | 0.98 | 0.98 | 3.9 |
| 5d | 0.98 | 0.98 | 1.95 | 0.49 | 0.49 | 0.98 | 0.49 | 0.49 | 0.98 | 1.95 |
| 5e | 7.8 | 7.8 | 3.9 | 3.9 | 3.9 | 7.8 | 7.8 | 7.8 | 7.8 | 7.8 |
| 5g | 3.9 | 0.98 | 1.95 | 3.9 | 3.9 | 1.95 | 1.95 | 3.9 | 3.9 | 3.9 |
| 6d | 1.95 | 0.98 | 1.95 | 0.98 | 0.98 | 1.95 | 0.98 | 0.98 | 1.95 | 3.9 |
| 7d | 1.95 | 0.98 | 1.95 | 0.49 | 0.49 | 0.98 | 0.49 | 0.49 | 1.95 | 3.9 |
| 7e | 7.8 | 15.6 | 3.9 | 7.9 | 7.8 | 15.6 | 15.6 | 7.8 | 7.8 | 7.8 |
| 7f | 0.98 | 0.98 | 0.98 | 0.98 | 0.49 | 0.98 | 0.98 | 0.98 | 0.98 | 31.3 |
| 7g | 3.9 | 1.95 | 3.9 | 0.98 | 0.98 | 1.95 | 1.95 | 3.9 | 3.9 | 7.8 |
| 8d | 0.98 | 0.98 | 0.98 | 0.49 | 0.24 | 0.98 | 0.49 | 0.49 | 0.98 | 1.95 |
| 8e | 7.8 | 7.8 | 7.8 | 7.8 | 3.9 | 15.6 | 7.8 | 15.6 | 7.8 | 7.8 |
| 8f | 0.98 | 0.98 | 1.95 | 0.98 | 0.49 | 0.98 | 0.98 | 0.98 | 0.98 | 31.3 |
| 8g | 1.95 | 1.95 | 1.95 | 0.98 | 0.49 | 1.95 | 0.98 | 0.98 | 1.95 | 31.3 |
| AmB | 0.98 | 0.98 | 1.95 | 1.95 | 1.95 | 0.98 | 0.98 | 1.95 | 1.95 | 1.95 |

From the remaining set of compounds tested, compounds 2c, 2d, 2e, 2f, 2j, 3c, 3d, 3e, 3g, 4d, 5c, 5e, 5g, 6d, 7d, 7g, 9c, and 9g exhibited excellent to good activity (0.12-7.8 j·tg mL⁻¹) against all ten C. auris strains tested. Compounds 7f, 8f, and 8g exhibited excellent activity (0.49-1.95 j·tg mL⁻¹) against all of the strains tested, with the exception of compounds 7f, 8f, and 8g against C. auris strain AR Bank #390. Similar to the result observed in Table 1A, compounds 1b, 1j, 7e, and 8e displayed poor activity against most of the C. auris strains tested.

Next, the compounds from the entire series 2 were tested and members of series 9 were selected against a panel of ten additional other fungal strains, including three Candida duobushaemulonii strains (AR Bank #391, AR Bank #392, and AR Bank #394), two Candida haemulonii strains (AR Bank #393 and AR Bank #395), two Saccharomyces cerevisiae strains (AR Bank #399 and AR Bank #400), and one each of the following strains: Kodameae ohmeri (AR Bank #396), Candida krusei (AR Bank #397), and Candida lusitaniae (AR Bank #398) (Table 2A). Monohydrazides 2a, 2b, 2e, 2f, 2 h, 2i, 9b, and 9f displayed excellent activity (0.06-1.95 j·tg mL⁻¹) against all ten additional strains tested, whereas the remaining compounds 2c, 2d, 2g, 2j, 9c, 9c, and 9g exhibited excellent to good activity (0.06-7.8 j·tg mL⁻¹) against all ten strains. Overall, as shown in Table 2A and 2B, the most active monohydrazides displayed excellent activity against a panel of ten C. auris (AR Bank #381-390) and ten other fungal strains (AR Bank #391-400).

### Example 54: Structure-Activity Relationship (SAR) Analysis

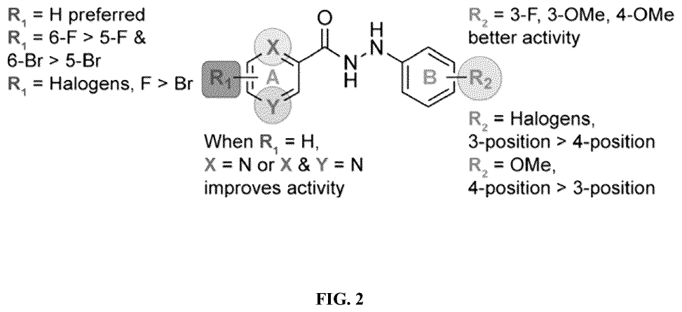

The identity and the substitution pattern of the substituent(s) on rings A (R1) and B (R2) had a considerable influence on the activity of monohydrazides 1a-9j (summarized in FIG. 2). The effect of the R₂ substituents were first investigated, while keeping R1 constant (i.e., comparing the monohydrazides within each series). For series 1, when R1=2,4-diF (FIG. 1), the introduction of either 4-OMe (1f), 3-F (1a), or 4-F (1d) as R₂ substituents resulted in better antifungal activity than other substituents (i.e., 4-Cl (1c), 3,5-diF (1i), and 3,5-diCl (1j)). In the case of series 2 (R1=H; FIG. 1), compounds 2a, 2i, 2b, 2g, 2d, and 2e that displayed excellent activity had 3-F, 3,5-diF, 3-Cl, 2,4-diF, 4-F, and 4-Cl as R₂ substituents. When comparing series 3 and 4 (R1=5-F and 5-Br, respectively; FIG. 1), the most active monohydrazides in each series (i.e., (3f, 3c, and 3a) and (4c, 4a, and 4f)) had (4-OMe, 3-OMe, and 3-F) and (3-OMe, 3-F, and 4-OMe) as R₂ substituents, respectively. For R1 groups=6-F and 6-Br (series 5 and series 6; FIG. 1), the most active compounds in each series had R₂ substituents as 4-OMe (5f), 4-F (5d), and 3-OMe (5c) and 3-OMe (6c), 4-OMe (6f), and 3-F (6a), respectively. In the case of series 7 and 8 (R1=3,5-diF and 3,6-diF, respectively; FIG. 1), compounds 7d, 7a, and 7f (from series 7) and compounds 8d, 8f, and 8a (from series 8) that displayed excellent activity had 4-F, 3-F, and 4-OMe as well as 4-F, 4-OMe, and 3-F, as R₂ substituents. For series 9 (R1=H; FIG. 1), the R₂ substituents 3-OMe (9c), 4-OMe (9f), 2,4-diF (9g), 3-F (9a), and 4-F (9d) resulted in better activity compared to other substituents (i.e., 3-Cl (9b), 4-Cl (9c), 2,5-diF (9h), 3,5-diF (9i), and 3,5-diCl (9j)). In sum, in each series (1-9), the most of the active monohydrazides had either 3-F (a), 3-OMe (c), or 4-OMe (f) as R₂ substituents.

Next, the effect of varying the R1 substituent was explored while keeping R₂ constant (i.e., comparing all “a” compounds 1a-9a, then all “b” compounds 1b-9b, etc.). The monohydrazides displaying the best antifungal activity generally had H (series 2 where X═N and Y═C as well as series 9 where X═N and Y═N) as an R1 substituent. For compounds with R2=3-F (a), the most active compounds (from most to least active) were 2a (R1=H, X═N, Y ═C), 9a (R1=H, X═N, Y═N), 7a (R1=3,5-diF), and 8a (R1=3,6-diF), respectively. For compounds with R2=3-Cl (b), the introduction of H as an R1 substituent resulted in compounds 2b and 9b with a better overall antifungal activity. The most active compounds amongst monohydrazides with R2=3-OMe (c), compounds 9c, 2c, 3c, and 5c, had H (X═N, Y═C), H (X═N, Y═N), 5-F, and 6-F as R1 substituents, respectively. For compounds with R2=4-F (d), the most active compounds 2d, 8d, 5d, and 9d possessed H (X═N, Y═C), 3,6-diF, 6-F, and H (X═N, Y═N) as R1 substituents, respectively. For compounds with R2=4-Cl (c), the most active compounds 2e and 9e possessed H as R1 substituents. For compounds with R2=4-OMe (f), the most active compounds had 6-F (5f), H (2f and 9f), 2,4-diF (1f), and 5-F (3f) as R1 substituents. For monohydrazides with R2=2,4-diF (g), the presence of H and 5-F as R₁ substituents resulted in compounds 9g, 2g, and 3g with better overall antifungal activity than those with other R1 substituents. Similar activity profiles were observed for compounds with R2=2,5-diF (h), R2=3,5-diF (i), and R2=3,5-diCl (j). The introduction of H (X═N, Y═C) and H (X═N, Y═N) as R1 substituents resulted in compounds 2 h, 9h, 2i, 9i, 2j, and 9j with better overall activity. In general, the monohydrazides with the best overall antifungal activity with diverse R₂ groups had H (series 2 and 9) as an R1 substituent.

For further in-depth analysis of the antifungal activity, the effect of regioisomers on ring A was explored by comparing series 3 (R1=5-F) with series 5 (R1=6-F) and series 4 (R1=5-Br) with series 6 (R1=6-Br). Compounds 5d (R2=4-F), 5c (R2=4-Cl), and 5f (R2=4-OMe) performed better than their counterparts 3d (R2=4-F), 3c (R2=4-Cl), and 3f (R2=4-OMe), whereas compounds 3a (R2=3-F) and 3g (R2=2,4-diF) displayed better activity compared to 5a (R2=3-F) and 5g (R2=2,4-diF). While comparing series 4 (R1=5-Br) with series 6 (R1=6-Br), compounds 6d (R2=4-F), 6f (R2=4-OMe), and 6g (R2=2,4-diF) were found to be better antifungals than their counterparts 4d (R2=4-F), 4f (R2=4-OMe), and 4g (R2=2,4-diF). From the data reported above, the superiority of series 5 and 6 (R1=6-F and R1=6-Br) over series 3 and 4 (R1=5-F and R1=5-Br) was found. For disubstituted monohydrazides (series 7 and 8), compounds 7a (R2=3-F) and 7f (R2=4-OMe) exhibited similar activity to 8a (R2=3-F) and 8f (R2=4-OMe), whereas compounds 8c (R2=3-OMe), 8d (R2=4-F), and 8g (R2=2,4-diF) were better than their counterparts from series 7. These observations point to fact that having a substituent at the 6-position of ring A substantially increases the activity of the compounds.

Next, the impact of a halogen identity on antifungal activity was evaluated by comparing series 3 and 4 (5-F vs. 5-Br) as well as series 5 and 6 (6-F vs. 6-Br). For every R₂ substituents, compounds from series 3 (3a, 3c, 3d, 3f, and 3g) performed significantly better than compounds from series 4 (4a, 4c, 4d, 4f, and 4g). A similar trend was observed in the case of series 5 and 6. Compounds 5a (R2=3-F), 5c (R2=3-OMe), 5d (R2=4-F), 5f (R2=4-OMe), and 5g (R2=2,4-diF) performed better than their counterparts 6a (R2=3-F), 6c (R2=3-OMc), 6d (R2=4-F), 6f (R2=4-OMc), and 6g (R2=2,4-diF). From these observations, a fluorine is preferred over a bromine as a R1 substituent for antifungal activity.

Finally, the effect of the position of the R₂ substituents on ring B (i.e., 3-vs. 4-position) was explored within each series for the entire nine series of compounds (1a vs. 1d, 1c vs. 1f, and 1b vs. 1e, etc.). For monohydrazides where R₂ substituents are halogens (3-F (a) and 4-F (d) or 3-Cl (b) and 4-Cl (c)), in general better antifungal activity was observed for 3-position isomers over 4-position isomers. Indeed, for compounds with R2=3-F (a) and 4-F (d), 1a, 2a, 3a, 4a, and 6a performed better than their counter parts 1d, 2d, 3d, 4d, and 6d. Similarly, for compounds with R2=3-Cl (b) and 4-Cl (c), 1b, 2b, and 9b displayed better antifungal activity than compounds 1e, 2e, and 9c. However, for compounds with R₂=OMe (1c-9c with 3-OMe vs. 1f-9f with 4-OMe), the 4-position isomers were better antifungals when compared to the 3-position isomers. Compounds 1f, 3f, 5f, 7f, and 8f displayed better activity than their counter parts 1c, 3c, 5c, 7c, and 8c. Next, the effect of a halogen identity on ring B (i.e., F (a and d) vs. Cl (b and c)) on antifungal activity was explored. When comparing compounds with R2=4-F (d) and R2=4-Cl (c), the presence of a fluorine (compounds 1d, 3d, 5d, 7d, 8d, and 9d) is preferred over that of a chlorine atom (1e, 3e, 5e, 7e, 8c, and 9c). However, when comparing compounds with R2=3-F and R2=3-Cl (1a vs. 1b, 2a vs. 2b, and 9a vs. 9b), differences in antifungal activity were not observed.

### Example 55: In Vitro Cytotoxicity Assay

Having established the excellent activity of the monohydrazides against most fungal strains tested, the toxicity of these compounds towards mammalian cell lines was considered. The toxicity profile of compounds 2a, 2b, 2d, 2g, 2i, 9a, 9b, 9c, 9f, 9g, was investigated as well as controls AmB and VRC (within a concentration range of 0.12-31.3 j·tg mL⁻¹) against three mammalian cell lines: J774A.1, HEK-293, and HepG2 (FIG. 3A-3C). At the highest concentration tested (31.3 j·tg mL⁻¹), none of the monohydrazides used in this study displayed toxicity against the J774A.1 and HEK-293 cell lines. Against HepG2, compounds 2a, 2b, 2d, 2g, 9b, 9c, 9f, and 9g exhibited no toxicity up to 31.3 j·tg mL⁻¹. However compounds 2i and 9a displayed minimal toxicity (89% and 94% cell survival, respectively) at a concentration of 31.3 j·tg mL⁻¹ against HepG2. Considering the excellent antifungal MIC values for these compounds, these cytotoxicity data provides us with a reasonable therapeutic window.

### Example 56: Physicochemical Properties and in Silico ADMET Evaluation

A theoretical evaluation of physicochemical and absorption, distribution, metabolism, excretion, and toxicity (ADMET) properties can provide important information regarding the drug likeness of the synthesized monohydrazides. The physicochemical and ADMET properties of potent compounds 2a-2j, 9a-9c, 9f, and 9g was computed using ADMETlab 2.0.^{54,55} All compounds assessed satisfied the Lipinski's rule of five and had a total polar surface area (TPSA) in the range of 50-77 Å, which suggested good cell membrane permeability. Additionally, the lipophilicity (log P) and solubility (log S) scores for the compounds screened were within the acceptable range (−3.6 to −1.6 for log S and 1 to 2.5 for log P) (Table 3). Next, the effect of these monohydrazides on the heart was assessed by virtually screening them for inhibition of the human ether-à-go-go-related (hERG) potassium ion channel. The prediction suggested that none of the compounds would be hERG blockers. When virtually analyzed for hepatotoxicity, the compounds were predicted to be high risk for drug-induced liver injury. The compounds from series 2 (2a-2j) were predicted to be moderately carcinogenic, whereas compounds 9a-9c, 9f, and 9g were predicted to be non-carcinogenic. The in silico predictions revealed that all the compounds screened had acceptable human intestinal absorption (HIA) values and, except for compounds 9a, 9c, and 9g, the remaining compounds were predicted to have higher blood-brain barrier (BBB) penetration. Interestingly, the monohydrazides screened were predicted to be neither P-glycoprotein (Pgp) inhibitors nor Pgp substrates. Plasma protein binding (PPB), volume distribution (VD), and clearance (CL) were also evaluated by ADMETlab 2.0. Except for compound 2j, all compounds had a <90% PPB values, suggesting high therapeutic index for these compounds. The predicted VD values for all the compounds were within the optimal range of 0.04-20 L kg-1. As far as clearance (CL) is concerned, from all the compounds virtually tested, only compounds 2e and 2j were predicted to have low clearance (Table 4)

TABLE 3
Physicochemical properties of selected compounds 2a-2j,
9a-9c, 9f, and 9g used in this study.

| Cpd # | TPSA | Log S | Log P | Lipinski |
|---|---|---|---|---|
| 2a | 54.02 | −2.212 | [symbol] | Yes |
| 2b | 54.02 | −2.839 | 2.505 | Yes |
| 2c | 63.25 | −2.172 | 1.844 | Yes |
| 2d | 54.02 | −2.325 | [symbol] | Yes |
| 2e | 54.02 | −2.925 | [symbol] | Yes |
| 2f | 63.25 | −2.227 | 1.773 | Yes |
| 2g | 54.02 | −2.739 | 2.016 | Yes |
| 2h | 54.02 | −2.874 | [symbol] | Yes |
| 2i | 54.02 | [symbol] | 2.073 | Yes |
| 2j | 54.02 | −3.59 | 3.309 | Yes |
| 9a | 66.91 | [symbol] | 1.134 | Yes |
| 9b | 66.91 | −2.404 | 1.781 | Yes |
| 9c | 76.41 | −1.649 | 1.061 | Yes |
| 9f | 76.41 | −1.782 | 1.0 | Yes |
| 9g | 66.91 | −2.179 | 1.168 | Yes |

TPSA = Topological polar surface area
Log S = log of aqueous solubility
Log P = Log of octanol/water partition coefficient [symbol]
indicates data missing or illegible when filed

TABLE 4
Predicted ADME/T properties of selected compounds 2a-2j, 9a-9c, 9f, and 9g used in this study.

| Cpd # | HIA | Pgp-inhibitor | Pgp-substrate | BBB | PPB % | VD | CL | hERG Blockers | Carcinogenicity | H-HT |
|---|---|---|---|---|---|---|---|---|---|---|
| 2a | 0.007 | 0.002 | 0.003 | 0.997 | 72.03 | 0.643 | 7.206 | 0.006 | 0.88 | 0.923 |
| 2b | 0.008 | 0.004 | 0.003 | 0.998 | 89.95 | 0.721 | 4.406 | 0.005 | 0.668 | 0.776 |
| 2c | 0.008 | 0.002 | 0.003 | 0.998 | 75.68 | 7.354 | 7.354 | 0.008 | 0.844 | 0.912 |
| 2d | 0.006 | 0.002 | 0.003 | 0.995 | 79.56 | 0.551 | 6.19 | 0.004 | 0.879 | 0.927 |
| 2e | 0.007 | 0.004 | 0.003 | 0.997 | 86.84 | 0.664 | 4.074 | 0.004 | 0.726 | 0.725 |
| 2f | 0.000 | 0.008 | 0.003 | 0.996 | 72.03 | 0.643 | 7.206 | 0.006 | 0.875 | 0.897 |
| 2g | 0.004 | 0.000 | 0.001 | 0.974 | 84.72 | 0.667 | 6.236 | 0.004 | 0.907 | 0.976 |
| 2h | 0.004 | 0.009 | 0.002 | 0.965 | 86.51 | 0.71 | 5.986 | 0.006 | 0.894 | 0.98 |
| 2i | 0.004 | 0.012 | 0.003 | 0.976 | 88.84 | 0.641 | 6.39 | 0.01 | 0.885 | 0.957 |
| 2j | 0.006 | 0.018 | 0.004 | 0.973 | 97.55 | 0.895 | 3.882 | 0.008 | 0.608 | 0.778 |
| 9a | 0.007 | 0.0 | 0.006 | 0.646 | 55.85 | 0.92 | 7.903 | 0.013 | 0.178 | 0.944 |
| 9b | 0.01 | 0.001 | 0.01 | 0.725 | 66.37 | 0.945 | 7.134 | 0.014 | 0.128 | 0.862 |
| 9c | 0.008 | 0.0 | 0.006 | 0.649 | 52.21 | 0.947 | 9.438 | 0.016 | 0.221 | 0.935 |
| 9f | 0.007 | 0.0 | 0.008 | 0.734 | 48.59 | 0.901 | 9.231 | 0.016 | 0.193 | 0.932 |
| 9g | 0.005 | 0.001 | 0.002 | 0.534 | 63.50 | 1.049 | 8.017 | 0.011 | 0.204 | 0.98 |

HIA = Human intestinal absorption
Pgp = P-glycoprotein
BBB = Blood-brain barrier
PPB = Plasma protein binding (Optimal <90%)
VD = Volume distribution (Optimal 0.04-20 L/Kg)
CL = Clearance (High: >15 mL/min/Kg; Moderate: 5-13 mL/min/Kg: Low: <5 mL/min/Kg)
hERG = human Ether-d-go-go-related
H-HT = Human hepatotoxicity
The output value represent probability.

The metabolic properties of potent compounds 2a-2j, 9a-9c, 9f, and 9g were also investigated in silico using SwissADME against the five isoforms of cytochrome P450 (CYP) monooxygenase (CYP1A2, CYP2C19, CYP2C9, CYP2D6, and CYP3A4). None of the compounds (with the exception of compound 2j against CYP2C19 and CYP2D6) were identified as possible inhibitors of CYP2C19, CYP2C9, CYP2D6, and CYP3A4. However, except for compound 2i, all compounds were predicted to inhibit CYP1A2 (Table 5)

TABLE 4
Predicted inhibition of cytochrome P450 (CYP) monooxygenase
by compounds 2a-2j, 9a-9c, 9f, and 9g.

| Cpd # | CYP1A2 | CYP2C19 | CYP2C9 | CYP2D6 | CYP3A4 |
|---|---|---|---|---|---|
| 2a | Yes | No | No | No | No |
| 2b | Yes | No | No | No | No |
| 2c | Yes | No | No | No | No |
| 2d | Yes | No | No | No | No |
| 2e | Yes | No | No | No | No |
| 2f | Yes | No | No | No | No |
| 2g | Yes | No | No | No | No |
| 2h | Yes | No | No | No | No |
| 2i | No | No | No | No | No |
| 2j | Yes | Yes | No | Yes | No |
| 9a | Yes | No | No | No | No |
| 9b | Yes | No | No | No | No |
| 9c | Yes | No | No | No | No |
| 9f | Yes | No | No | No | No |
| 9g | Yes | No | No | No | No |

### Example 57: Time-Kill Studies

In order to understand whether the monohydrazides prepared are either fungistatic (inhibit or prevent fungal growth) or fungicidal (kill the fungi), a time-kill assay was performed. Compound 2b (one of the most active compound from MIC studies) was tested at 1× and 4× MIC against C. albicans ATCC 10231 (strain A) to observe the dose-dependent effect on fungal growth (FIG. 4). In addition, the control AmB at 0.98 μg mL⁻¹ was evaluated against the same C. albicans strain A for comparison. The number of colony-forming units (CFU) was measured at 0, 3, 6, 9, 12, 18, and 24 h. At 4×MIC, compound 2b exhibited a fungistatic behavior.

### Example 58: Antibiofilm Activity

The biofilm is an important virulence contributor for pathogenic fungi, as the biofilm protects the fungi from drugs and shields the pathogens from the immune system.⁵⁶⁻⁵⁸ Biofilms are complex communities of one or more species of microorganisms surrounded by an extracellular matrix, which are not only attached to each other, but also to solid surfaces.^{59,60} Because of their complex structural features, antifungal agents do not penetrate the matrix well, and they cannot readily reaching the pathogens embedded in these multiple layers.⁶¹ Thus, biofilms are more resistant to antifungal drugs compared to plankatonic cells.^{62,63} Since the compounds from series 2 behaved exceptionally well during MIC studies, the ability of compounds 2a-2j, as well as control AmB were assessed against C. albicans ATCC 10231 (strain A) in both destruction of pre-formed biofilms and prevention of biofilm formation. A large amount of fungal cells were exposed to the monohydrazides at time 0 h to evaluate the effect of these compounds in the prevention of Candida biofilm formation (FIG. 5B). Monohydrazides 2g, 2 h, 2i, and 2j were not capable of preventing C. albicans ATCC 10231 (strain A) biofilm formation, whereas monohydrazides 2b, 2c, 2d, and 2e exhibited promising activity (1.96-3.9 j·tg mL⁻¹) against biofilm formation of the same fungal strain. Moderate activity (7.8 j·tg mL⁻¹) was observed for compounds 2a and 2f in prevention of biofilm formation assays. Amongst all the monohydrazides tested, compounds 2c and 2d were the most promising with their ability to inhibit biomass formation at 1.96 j·tg mL⁻¹ (8 to 16-fold greated then MIC values). Although some of these monohydrazides were able to prevent biofilm formation, none of these compounds had any effect in terms of the disruption of already pre-formed biofilms (FIG. 5A).

### Example 59: Resistance Development Studies

In order to evaluate the potential of fungi to develop resistance to monohydrazides, C. albicans ATCC 10231 (strain A) were repeatedly exposed to compounds 2a and 2b at ½×MIC to simulate fungal drug exposure in a clinical setting (FIG. 6). The fungi that grew at the highest concentration of compounds (½×MIC) were grown and used as the starting culture for a successive MIC determination. This assay was repeated for a total of 15 MIC determinations (original+14 exposures). While normal variations in MIC values occurred, no significant changes in MIC values were observed as the MIC values remained within 8-fold of the original MIC value. Considering the generally long duration of treatment with antifungal drugs, this is a promising result that suggests that a fungal strain is not likely to develop resistance to the monohydrazides, even after repeated exposures.

### Example 60: Summary

As described in these studies, sixty-four unique substituted monohydrazides (1a-9j) were synthesized with different R₁ and R₂ substituents on rings A and B. A detailed antifungal activity study was performed on these compounds against a panel of various C. albicans, non-albicans Candida, and non-Candida fungal strains. From this detailed SAR study, compounds 1f, 2a, 2b, 2c, 2d, 2e, 2f, 2g, 2 h, 2i, 2j, 3f, 5d, 5f, 8d, 9b, 9c, 9f, and 9g exhibited broad spectrum activity based on their MIC values. The activity of these compounds was either comparable or superior to AmB against most of the strains tested.

The entire series 2, and some of the representative compounds from series 1 and 3-9 were further tested against a panel of ten C. auris and ten other fungal strains. The monohydrazides 2a, 2b, 2g, 2 h, 2i, 5d, 8d, 9b, and 9g displayed excellent activity against the ten C. auris strains tested, and compounds 2a, 2b, 2e, 2f, 2 h, 2i, 9b, and 9g exhibited excellent activity against the ten other fungal strains.

The safety profile of the monohydrazides was explored by investigating compounds 2a, 2b, 2d, 2g, 2i, 9a, 9b, 9c, 9f, and 9g, as well as controls AmB and VRC against three mammalian cell lines: J774A.1, HEK-293, and HepG2. The entire panel of compounds investigated exhibited minimal to no toxicity against the three different mammalian cell lines tested.

Compound 2b was observed to be fungistatic at and/or above its MIC value against C. albicans ATCC 10231 (strain A) in a time-kill assay. Compounds 2a, 2b, 2c, 2d, 2e, 2f, 2g, 2 h, 2i, 2j, as well as control AmB were assessed against C. albicans ATCC 10231 (strain A) in both destruction of pre-formed biofilms and prevention of biofilm formation. These monohydrazides were able to prevent the formation of biofilm against strain A. When C. albicans ATCC 10231 (strain A) was repeatedly exposed to the compounds 2a and 2b over 15 passages, no resistance was developed against these compounds.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1 G. D. Brown, D. W. Denning and S. M. Levitz, Science, 2012, 336, 647.
2 T. Roemer and D. J. Krysan, Cold Spring Harbor Perspect. Med., 2014, 4, a019703.
3 G. Karavalakis, E. Yannaki and A. Papadopoulou, J. Fungi, 2021, 7, 451.
4 A. Casadevall, Annu. Rev. Immunol., 2022, 40, 121-141.
5 D. Yu and Z. Liu, Front. Microbiol., 2022, 13, 988734.
6 M. Mei-Sheng Riley, Crit. Care Nurs. Clin. N. Am., 2021, 33, 395-405.
7 S. Lakoh, P. S. Kamudumuli, R. O. S. Penney, S. M. Haumba, J. N. Jarvis, A. J. Hassan, N. L. E. Moudoute, B. K. Ocansey, S. Izco, S. Kipkerich, J. Sacarlal, A. T. Awopeju, N. P. Govender, C. I. M. Munyanji, K. Guyguy, E. Orefuwa and D. W. Denning, Lancet Infect. Dis., 2022, DOI: 10.1016/S1473-3099 (22) 00656-9, S1473-3099 (1422) 0065-1479.
8 D. Forno, B. Samayoa, N. Medina, E. Arathoon, C. R. Mejia, R. Gordillo, R. Cedillos, J. Rodas, A. Ahlquist Cleveland, T. Chiller and D. H. Caceres, Mycoses, 2021, 64, 1563-1570.
9 M. C. Fisher, A. Alastruey-Izquierdo, J. Berman, T. Bicanic, E. M. Bignell, P. Bowyer, M. Bromley, R. Bruggemann, G. Garber, O. A. Cornely, S. J. Gurr, T. S. Harrison, E. Kuijper, J. Rhodes, D. C. Sheppard, A. Warris, P. L. White, J. Xu, B. Zwaan and P. E. Verweij, Nat. Rev. Microbiol., 2022, 20, 557-571.
10 A. McDermott, Proc. Natl. Acad. Sci. U.S.A., 2022, 119, e2217948119.
11 N. Robbins, T. Caplan and L. E. Cowen, Annu. Rev. Microbiol., 2017, 71, 753-775.
12 D. W. Denning, Eur. J. Hosp. Pharm., 2022, 29, 109-112.
13 J. Berman and D. J. Krysan, Nat. Rev. Microbiol., 2020, 18, 319-331.
14 H. Du, J. Bing, T. Hu, C. L. Ennis, C. J. Nobile and G. Huang, PLOS Pathog., 2020, 16, e1008921.
15 E. S. Spivak and K. E. Hanson, J. Clin. Microbiol., 2018, 56, e10588-e10517.
16 M. Kordalewska and D. S. Perlin, Front. Microbiol., 2019, 10, 1918.
17 K. Forsberg, K. Woodworth, M. Walters, E. L. Berkow, B. Jackson, T. Chiller and S. Vallabhaneni, Med. Mycol., 2019, 57, 1-12.
18 D. W. Eyre, A. E. Sheppard, H. Madder, I. Moir, R. Moroney, T. P. Quan, D. Griffiths, S. George, L. Butcher, M. Morgan, R. Newnham, M. Sunderland, T. Clarke, D. Foster, P. Hoffman, A. M. Borman, E. M. Johnson, G. Moore, C. S. Brown, A. S. Walker, T. E. A. Peto, D. W. Crook and K. J. M. Jeffery, N. Engl. J. Med., 2018, 379, 1322-1331.
19 A. Cortegiani, G. Misseri, A. Giarratano, M. Bassetti and D. Eyre, Crit Care., 2019, 23, 150.
20 B. O'Brien, J. Liang, S. Chaturvedi, J. L. Jacobs and V. Chaturvedi, Lancet Microbe, 2020, 1, e193-e194.
21 Increasing threat of spread of antimicrobial-resistant fungus in healthcare facilities. Centers for Disease Control and Prevention Press release, Mar. 20, 2023.
22 N. Robbins, G. D. Wright and L. E. Cowen, Microbiol. Spectrum, 2016, 4.
23 J. R. Perfect, Nat. Rev. Drug Discovery, 2017, 16, 603-616.
24 Y. Lee, E. Puumala, N. Robbins and L. E. Cowen, Chem. Rev., 2021, 121, 3390-3411.
25 S. E. Murphy and T. Bicanic, Front. Cell. Infect. Microbiol., 2021, 11, 759408.
26 M. Doman and K. Banyai, Front. Microbiol., 2022, 13, 919501.
27 M. Hoenigl, D. Seidel, R. Sprute, C. Cunha, M. Oliverio, G. H. Goldman, A. S. Ibrahim and A. Carvalho, Nat. Microbiol., 2022, 7, 1127-1140.
28 A. A. Shishido, M. Mathew and J. W. Baddley, Curr. Fungal Infect. Rep., 2022, 16, 87-97.
29 A. Amin, A. Vartanian, N. Poladian, A. Voloshko, A. Yegiazaryan, A. L. Al-Kassir and V. Venketaraman, Infect. Dis. Rep., 2021, 13, 1018-1035.
30 C. M. Morel, R. A. Alm, C. Ardal, A. Bandera, G. M. Bruno, E. Carrara, G. L. Colombo, M. E. A. de Kraker, S. Essack, I. Frost, B. Gonzalez-Zorn, H. Goossens, L. Guardabassi, S. Harbarth, P. S. Jorgensen, S. S. Kanj, T. Kostyanev, R. Laxminarayan, F. Leonard, G. L. Hara, M. Mendelson, M. Mikulska, N. T. Mutters, K. Outterson, J. R. Bano, E. Tacconelli, L. Scudeller and G. A.-O. Network, Antimicrob. Resist. Infect. Control., 2020, 9, 187.
31 R. Smith and J. Coast, BMJ, 2013, 346, f1493.
32 E. Rayens and K. A. Norris, Open Forum Infect. Dis., 2022, 9, ofab593.
33 A. M. Rauseo, A. Coler-Reilly, L. Larson and A. Spec, Open Forum Infect. Dis., 2020, 7, ofaa016.
34 J. H. Kim, L. W. Cheng and K. M. Land, Pharmaceuticals, 2022, 15, 787.
35 X. Cui, L. Wang, Y. Lu and C. Yue, J. Infect. Public Health, 2022, 15, 986-1000.
36 N. Thamban Chandrika and S. Garneau-Tsodikova, Chem. Soc. Rev., 2018, 47, 1189-1249.
37 H. X. Ngo, S. Garneau-Tsodikova and K. D. Green, MedChemComm, 2016, 7, 1285-1306.
38 N. Thamban Chandrika and S. Garneau-Tsodikova, MedChemComm, 2016, 7, 50-68.
39 K. C. Howard, E. K. Dennis, D. S. Watt and S. Garneau-Tsodikova, Chem. Soc. Rev., 2020, 49, 2426-2480.
40 N. Thamban Chandrika, S. K. Shrestha, H. X. Ngo and S. Garneau-Tsodikova, Bioorg. Med. Chem., 2016, 24, 3680-3686.
41 N. Thamban Chandrika, S. K. Shrestha, H. X. Ngo, O. V. Tsodikov, K. C. Howard and S. Garneau-Tsodikova, J. Med. Chem., 2018, 61, 158-173.
42 N. Thamban Chandrika, S. K. Shrestha, H. X. Ngo, K. C. Howard and S. Garneau-Tsodikova, Bioorg. Med. Chem., 2018, 26, 573-580.
43 N. Thamban Chandrika, S. K. Shrestha, N. Ranjan, A. Sharma, D. P. Arya and S. Garneau-Tsodikova, ACS Infect. Dis., 2018, 4, 196-207.
44 M. Y. Fosso, S. K. Shrestha, K. D. Green and S. Garneau-Tsodikova, J. Med. Chem., 2015, 58, 9124-9132.
45 S. K. Shrestha, M. Y. Fosso and S. Garneau-Tsodikova, Sci. Rep., 2015, 5, 17070.
46 S. K. Shrestha, M. Y. Fosso, K. D. Green and S. Garneau-Tsodikova, Antimicrob. Agents Chemother., 2015, 59, 4861-4869.
47 S. K. Shrestha, A. Garzan and S. Garneau-Tsodikova, Eur. J. Med. Chem., 2017, 133, 309-318.
48 E. K. Dennis, J. H. Kim, S. Parkin, S. G. Awuah and S. Garneau-Tsodikova, J. Med. Chem., 2020, 63, 2455-2469.
49 H. X. Ngo, S. K. Shrestha and S. Garneau-Tsodikova, ChemMedChem, 2016, 11, 1507-1516.
50 S. Y. L. Holbrook, A. Garzan, E. K. Dennis, S. K. Shrestha and S. Garneau-Tsodikova, Eur. J. Med. Chem., 2017, 139, 12-21.
51 S. K. Shrestha, L. M. Kril, K. D. Green, S. Kwiatkowski, V. M. Sviripa, J. R. Nickell, L. P. Dwoskin, D. S. Watt and S. Garneau-Tsodikova, Bioorg. Med. Chem., 2017, 25, 58-66.
52 N. Thamban Chandrika, E. K. Dennis, S. K. Shrestha, H. X. Ngo, K. D. Green, S. Kwiatkowski, A. G. Deaciuc, L. P. Dwoskin, D. S. Watt and S. Garneau-Tsodikova, Eur. J. Med. Chem., 2019, 164, 273-281.
53 N. Thamban Chandrika, E. K. Dennis, K. R. Brubaker, S. Kwiatkowski, D. S. Watt and S. Garneau-Tsodikova, ChemMedChem, 2021, 16, 124-133.
54 A. Daina, O. Michielin and V. Zoete, Sci. Rep., 2017, 7, 42717.
55 G. Xiong, Z. Wu, J. Yi, L. Fu, Z. Yang, C. Hsieh, M. Yin, X. Zeng, C. Wu, A. Lu, X. Chen, T. Hou and D. Cao, Nucleic Acids Res., 2021, 49, W5-W14.
56 R. Zarnowski, A. Noll, M. G. Chevrette, H. Sanchez, R. Jones, H. Anhalt, J. Fossen, A. Jaromin, C. Currie, J. E. Nett, A. Mitchell and D. R. Andes, Nat. Commun., 2021, 12, 6235.
57 C. B. Costa-Orlandi, J. C. O. Sardi, N. S. Pitangui, H. C. de Oliveira, L. Scorzoni, M. C. Galeane, K. P. Medina-Alarcon, W. Melo, M. Y. Marcelino, J. D. Braz, A. M. Fusco-Almeida and M. J. S. Mendes-Giannini, J. Fungi, 2017, 3, 22.
58 J. F. Kernien, B. D. Snarr, D. C. Sheppard and J. E. Nett, Front. Immunol., 2017, 8, 1968.
59 J. Chandra, D. M. Kuhn, P. K. Mukherjee, L. L. Hoyer, T. McCormick and M. A. Ghannoum, J. Bacteriol., 2001, 183, 5385-5394.
60 C. H. Kowalski, K. A. Morelli, D. Schultz, C. D. Nadell and R. A. Cramer, Proc. Natl. Acad. Sci. U.S.A., 2020, 117, 22473-22483.
61 E. Butassi, L. Svetaz, M. C. Carpinella, T. Efferth and S. Zacchino, Antibiotics, 2021, 10, 1053.
62 S. Fanning and A. P. Mitchell, PLOS Pathog., 2012, 8, e1002585.
63 E. Borghi, G. Morace, F. Borgo, R. Rajendran, L. Sherry, C. Nile and G. Ramage, Front. Microbiol., 2015, 6, 1077.
64 Green, K. D.; Thamban Chandrika, N.; Vu, L. Y.; Pang, A. H.; Tsodikov, O. V.; Garneau-Tsodikova, S. Aromatic hydrazides: A potential solution for Acinetobacter baumannii infections. Eur. J. Med. Chem. 2023, 249, 115165. DOI: 10.1016/j.ejmech.2023.115165.
65 (a) Clinical and Laboratory Standards Institute. Reference method for Broth Dilution Antifungal Susceptibility Testing of Yeasts-Approved Standard. (b) Clinical and Laboratory Standards Institute; Wayne, PA, USA: 2008. CLSI Document M27-A3. Clinical and Laboratory Standards Institute. Reference method for Broth Dilution Antifungal Susceptibility Testing of Filamentous Fungi: Approved Standard-Second Edition. Clinical and Laboratory Standards Institute; Wayne, PA, USA: 2008. CLSI Document M38-A2.
66 Pierce, C. G.; Uppuluri, P.; Tristan, A. R.; Wormley, F. L., Jr.; Mowat, E.; Ramage, G.; Lopez-Ribot, J. L. A simple and reproducible 96-well plate-based method for the formation of fungal biofilms and its application to antifungal susceptibility testing. Nat. Protoc. 2008, 3 (9), 1494-1500. DOI: 10.1038/nport. 2008.141.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound having the formula (I) or a pharmaceutically acceptable salt thereof: 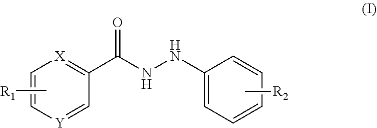
wherein
R₁ is selected from the group consisting of H, 5-Br, 6-Br, 5-F, 6-F, 2,4-diF, 3,5-diF, and 3,6-diF;
R₂ is selected from the group consisting of 3-OMe, 4-OMe, 3-Cl, 4-Cl, 3,5-diCl, 3-F, 4-F, 2,4-diF, 2,5-diF, and 3,5-diF; and
X and Y are independently selected from the group consisting of C and N.

2. The compound according to claim 1, having the formula selected from the group consisting of: 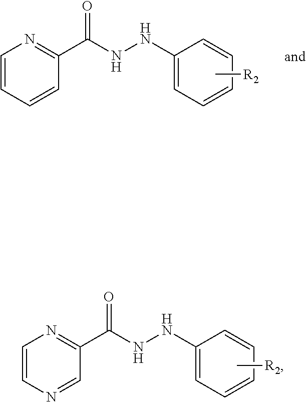
wherein R₂ is selected from the group consisting of 3-OMe, 4-OMe, 3-Cl, 4-Cl, 3,5-diCl, 3-F, 4-F, 2,4-diF, 2,5-diF, and 3,5-diF.

3. The compound according to claim 2, having the formula selected from the group

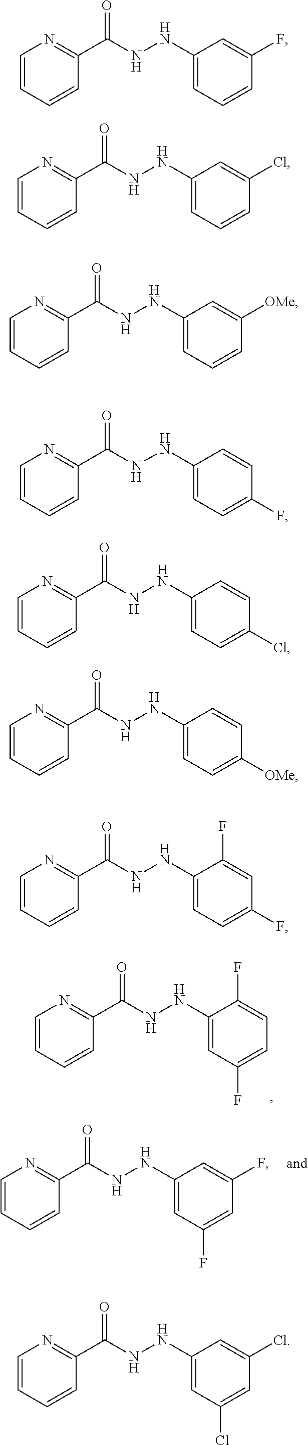

4. The compound according to claim 2, having the formula selected from the group

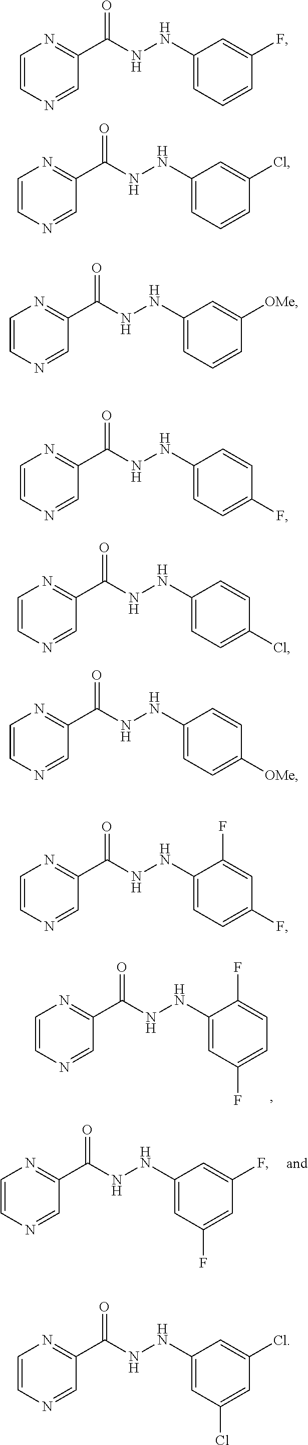

5. The compound according to claim 1, having the formula selected from the group 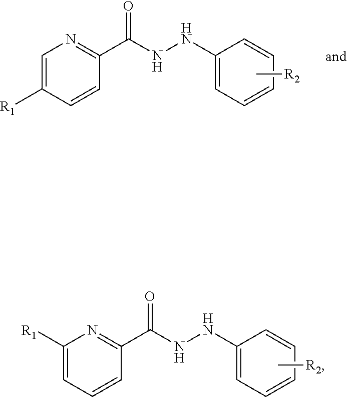
wherein
R₁ is selected from the group consisting of Br and F; and
R₂ is selected from the group consisting of 3-OMe, 4-OMe, 3-Cl, 4-Cl, 3,5-diCl, 3-F, 4-F, 2,4 diF, 2,5-diF, and 3,5-diF.

6. The compound according to claim 5, having the formula selected from the group

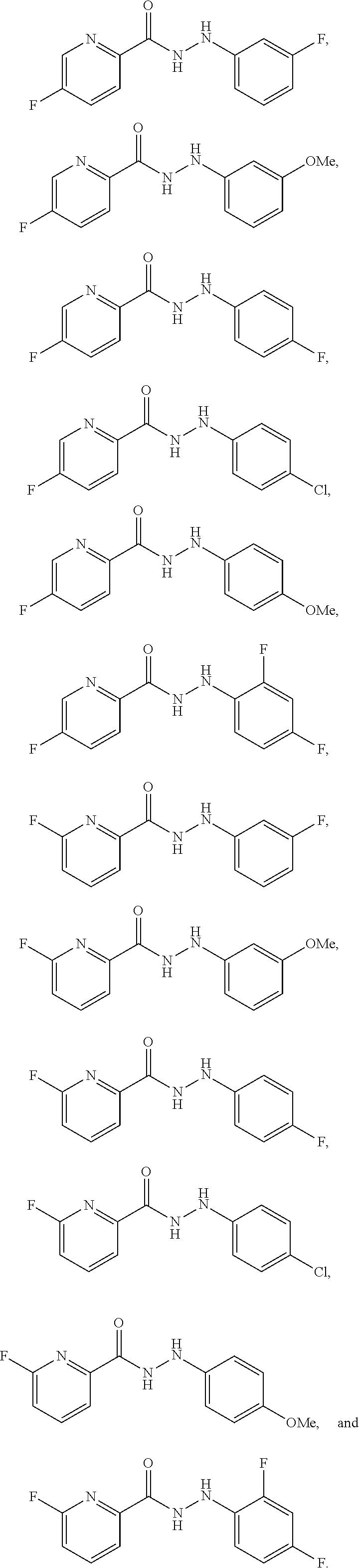

7. The compound according to claim 5, having the formula selected from the group

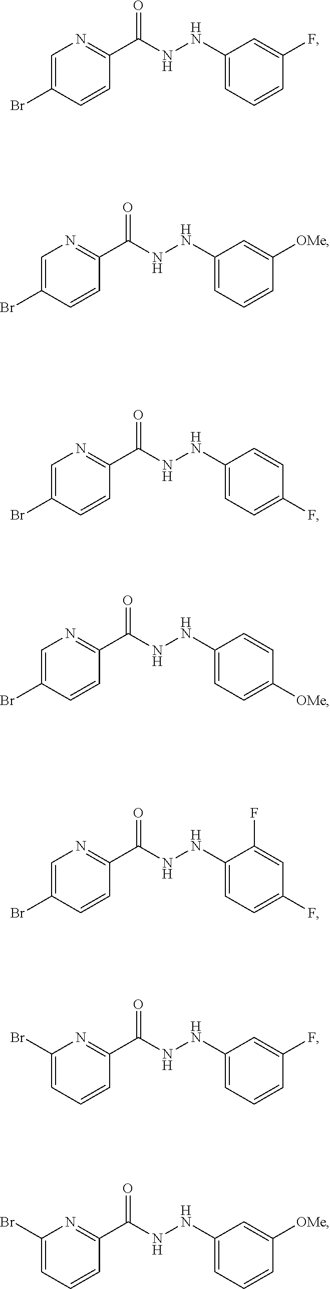

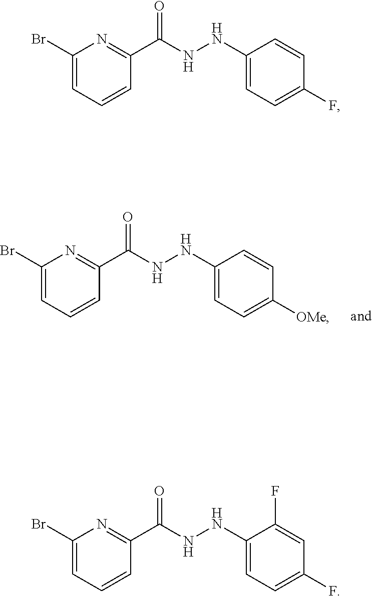 [SMILES: O=C(NNc1ccc(F)cc1)c1cccc(Br)n1 ; COc1ccc(NNC(=O)c2cccc(Br)n2)cc1 ; O=C(NNc1ccc(F)cc1F)c1cccc(Br)n1]

8. The compound according to claim 1, having the formula selected from the group 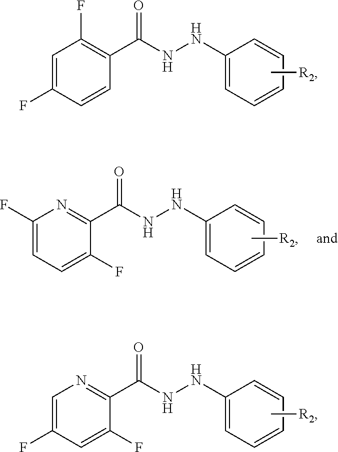
wherein R₂ is selected from the group consisting of 3-OMe, 4-OMe, 3-Cl, 4-Cl, 3,5-diCl, 3-F, 4-F, 2,4-diF, 2,5-diF, and 3,5-diF.

9. The compound according to claim 8, having the formula selected from the group

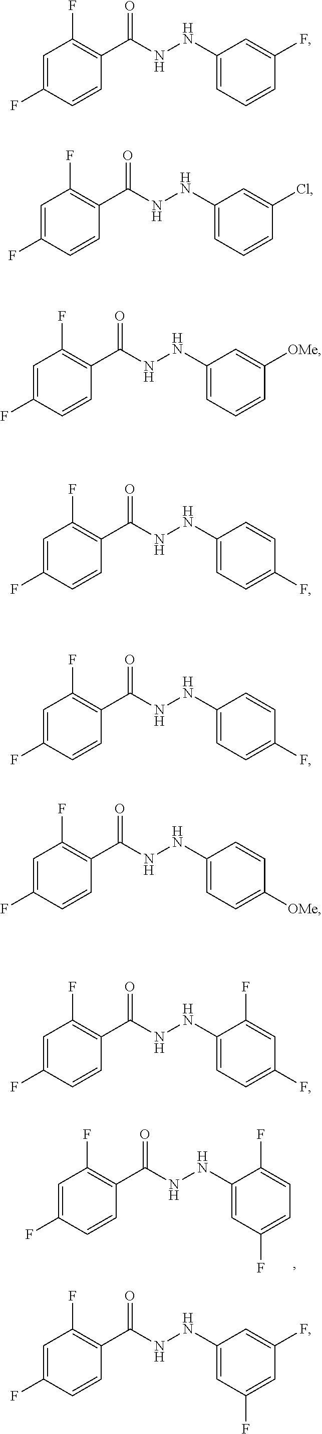

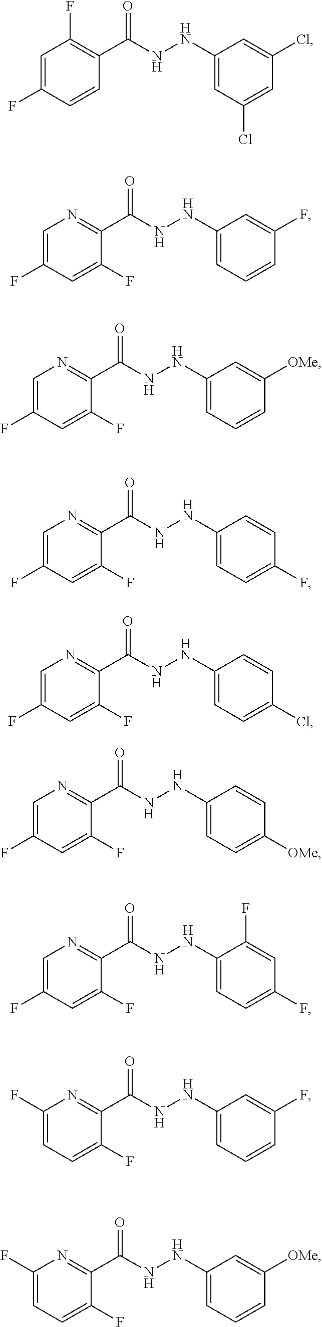

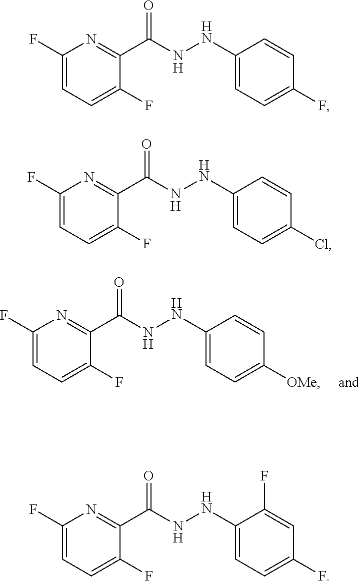

10. A composition comprising the compound of claim 1, and a suitable pharmaceutical carrier.

11. The composition of claim 10, comprising at least two compounds of claim 1.

12. A method of treating a fungal infection in a subject, comprising administering to the subject an effective amount of the compound of claim 1.

13. The method of claim 3, wherein the subject is a human subject.

14. The method of claim 3, wherein the subject is a plant or crop.
